(19) United States
(12) Patent Application Publication
(10) Pub. No.: US 2020/0254063 A1
(43) Pub. Date: Aug. 13, 2020

(54) METHODS AND COMPOSITIONS FOR MODULATION OF DYSREGULATED PLCG2 PHOSPHORYLATION

(71) Applicants: Anthony French, St. Louis, MO (US); Joshua Alinger, St. Louis, MO (US); Allison Throm, St. Louis, MO (US)

(72) Inventors: Anthony French, St. Louis, MO (US); Joshua Alinger, St. Louis, MO (US); Allison Throm, St. Louis, MO (US)

(73) Assignee: Washington University, St. Louis, MO (US)

(21) Appl. No.: 16/784,370

(22) Filed: Feb. 7, 2020

Related U.S. Application Data
(60) Provisional application No. 62/803,038, filed on Feb. 8, 2019.

(51) Int. Cl. A61K 38/20; A61P 31/22

(52) U.S. Cl. CPC A61K 38/2013; A61K 38/2086; A61P 31/22

(57) ABSTRACT

Among the various aspects of the present disclosure is the provision of methods and compositions for treating a PLCG2 hypophosphorylation-associated disease, disorder, or condition (e.g., juvenile dermatomyositis or recurrent herpesvirus) by administering cytokines, such as IL-2 or IL-15. Also provided herein are methods of modulating natural killer (NK) cell function, such as NK cell-mediated cytotoxicity or calcium flux.

### CROSS-REFERENCE TO RELATED APPLICATIONS

This application claims priority from U.S. Provisional Application Ser. No. 62/803,038 filed on 8 Feb. 2019, which is incorporated herein by reference in its entirety.

### STATEMENT REGARDING FEDERALLY SPONSORED RESEARCH OR DEVELOPMENT

Not applicable.

### MATERIAL INCORPORATED-BY-REFERENCE

Not applicable.

### FIELD OF THE INVENTION

The present disclosure generally relates to treatment of PLCG2 hypophosphorylation-associated diseases, disorders, and conditions.

### SUMMARY OF THE INVENTION

Among the various aspects of the present disclosure is the provision of a methods and compositions for treating a PLCG2 hypophosphorylation-associated disease, disorder, or condition.

An aspect of the present disclosure provides for a method of restoring normal function and normal calcium flux in a dysfunctional natural killer (NK) cell comprising administering a therapeutically effective amount of a PLCG2 phosphorylation modulating agent (e.g., comprising a cytokine or IFNα or IFNγ blocking agent) to the NK cell, wherein the dysfunctional NK cell is from a subject having, suspected of having, or at risk of having a PLCG2 hypophosphorylation-associated disease, disorder, or condition.

In some embodiments, the dysfunctional NK cell has dysregulated PLCG2 signaling, PLCG2 haploinsufficiency, or PLCG2 hypophospohorylation.

In some embodiments, the PLCG2 hypophosphorylation-associated disease, disorder, or condition is PLCG2 haploinsufficiency.

In some embodiments, PLCG2 haploinsufficiency is a heterozygous loss-of-function mutation in PLCG2.

In some embodiments, the PLCG2 hypophosphorylation-associated disease, disorder, or condition is an autoimmune disease, an infectious disease, or an inflammatory disease, disorder, or condition.

In some embodiments, the PLCG2 hypophosphorylation-associated disease, disorder, or condition is juvenile dermatomyositis (JDM) or dermatomyositis (DM).

In some embodiments, the PLCG2 hypophosphorylation-associated disease, disorder, or condition is a viral infection.

In some embodiments, the viral infection is a herpesvirus, an adenovirus, a herpes simplex virus 1 (HSV1), or a cytomegalovirus (CMV).

In some embodiments, the viral infection is a herpesvirus infection and the herpesvirus infection is an unusually severe or recurrent herpesvirus infection.

In some embodiments, the subject has herpesvirus infection susceptibility or bacterial infection susceptibility.

In some embodiments, the dysfunctional NK cell is PLCG2 haploinsufficient and has a herpesvirus infection.

In some embodiments, the PLCG2 hypophosphorylation-associated disease, disorder, or condition is an inflammatory condition.

In some embodiments, the PLCG2 hypophosphorylation-associated disease, disorder, or condition is multiple sclerosis (MS), systemic lupus erythematosus (SLE), or rheumatoid arthritis (RA).

In some embodiments, the cytokine is IL-2 or IL-15.

In some embodiments, the cytokine is IL-2, IL-15, IL-18, IL-12, or CCL5.

In some embodiments, the PLCG2 phosphorylation modulation agent is an IFNα blocking agent or an IFNγ blocking agent.

In some embodiments, the PLCG2 phosphorylation modulating agent restores normal NK cell function and normal NK cell calcium flux in the dysfunctional NK cell.

In some embodiments, restoring normal function and normal calcium flux, in the dysfunctional NK cell, results in improved cytotoxicity of the dysfunctional NK cell or improved ability of the dysfunctional NK cell to suppress inappropriate adaptive immune responses compared to an untreated dysfunctional NK cell.

Another aspect of the present disclosure provides for a method of treating a subject in need thereof, comprising administering a PLCG2 phosphorylation modulating agent comprising a cytokine, wherein the subject has dysfunctional NK cells, wherein the subject has, is suspected of having, or is at risk of having a PLCG2 hypophosphorylation-associated disease, disorder, or condition.

In some embodiments, the dysfunctional NK cells have dysregulated

PLCG2 signaling, PLCG2 haploinsufficiency, or PLCG2 hypophospohorylation.

In some embodiments, the PLCG2 hypophosphorylation-associated disease, disorder, or condition is PLCG2 haploinsufficiency.

In some embodiments, the PLCG2 haploinsufficiency is a heterozygous loss-of-function mutation in PLCG2.

In some embodiments, the PLCG2 hypophosphorylation-associated disease, disorder, or condition is an autoimmune disease, an infectious disease, or an inflammatory disease, disorder, or condition.

In some embodiments, the PLCG2 hypophosphorylation-associated disease, disorder, or condition is juvenile dermatomyositis (JDM) or dermatomyositis (DM).

In some embodiments, the PLCG2 hypophosphorylation-associated disease, disorder, or condition is a viral infection.

In some embodiments, the viral infection is a herpesvirus, an adenovirus, a herpes simplex virus 1 (HSV1), or a cytomegalovirus (CMV).

In some embodiments, the viral infection is a herpesvirus infection and the herpesvirus infection is an unusually severe or recurrent herpesvirus infection.

In some embodiments, the subject has herpesvirus infection susceptibility or bacterial infection susceptibility.

In some embodiments, the dysfunctional NK cells are PLCG2 haploinsufficient and have a herpesvirus infection.

In some embodiments, the PLCG2 hypophosphorylation-associated disease, disorder, or condition is an inflammatory condition.

In some embodiments, the inflammatory condition is multiple sclerosis (MS), systemic lupus erythematosus (SLE), or rheumatoid arthritis (RA).

In some embodiments, the cytokine is IL-2 or IL-15.

In some embodiments, the cytokine is IL-2, IL-15, IL-18, IL-12, or CCL5.

In some embodiments, the PLCG2 phosphorylation modulation agent is an IFNα blocking agent or an IFNγ blocking agent.

In some embodiments, the PLCG2 phosphorylation modulating agent restores normal NK cell function and normal NK cell calcium flux in the dysfunctional NK cells.

In some embodiments, restoring normal NK cell function and normal NK cell calcium flux results in improved NK cell-mediated cytotoxicity, suppression of inappropriate adaptive immune responses, or reduced autoimmunity in the subject when compared to an untreated subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1E is a series of schematics and graphs showing familial NK cell deficiency is associated with novel heterozygous PLCG2 mutations. (A) Pedigree of family A; affected heterozygotes are shown in black symbols while unaffected or unevaluated heterozygotes are shown in gray or white, respectively. WT, wild-type allele. Sanger-sequencing chromatograms are shown for patients and unaffected relatives. Arrow denotes site of heterozygosity. (B) Pedigree and sanger sequencing of family B is displayed as in (A). (C) NK cell killing against K562 cells is quantified after incubation for four hours at a peripheral blood mononuclear cell (PBMC) to K562 ratio of 50:1. Upper and lower internal reference ranges are displayed with dashed lines. Each point represents a unique biologic replicate, either a separate blood draw (patients) or a separate individual (controls). (D) Flow cytometry evaluation of NK cells (CD3-CD56+) in healthy control (HC) versus patients A.I.2, A.II.3 and B.II.4. Percentage of NK cells in the lymphocyte gate is displayed. Internal normal NK cell reference range, 2.8% to 15.5%. (E) The location of variants, including previously reported PLAID (Exon 19 or 20-22 deletions) and APLAID (5707Y) variants are displayed with the domain structure of PLCG2. PH, Pleckstrin homology; nSH2, N-terminal Src Homology 2; cSH2, C-terminal SH2, SH3, Src Homology 3. Except where limited by patient sample availability (B.II.4 in C and D), all data is representative of two or more independent experiments. All error bars represent standard deviation from the mean.

FIG. 2A-FIG. 2F is a series of graphs and images showing loss-of-function mutations in PLCG2 and haploinsufficiency cause NK cell dysregulation. (A) PLCG2 phosphorylation in CD56^{Dim} NK cells after CD16 crosslinking is quantified by CyTOF, normalized to time 0 using an arcsinh transformation in three healthy controls (HO; two females, one male), two G595R patients (A.II.3 and A.I.2) and one L183F patient (B.II.4). A.R.M., Arcsinh ratio of mean. Error bars represents standard deviation from the mean. (B) Btk/ltk phosphorylation is shown as in A. (C) Total PLCG2 levels in CD56^{Dim} NK cells is quantified by flow cytometry in a healthy control versus G595R patients. Isotype, Isotype control (dotted black line). HC, healthy control (solid black line). A.II.3, G595R patient (solid red line). Patient A.I.2, G595R patient, (dotted red line). (D) Indo-1 calcium flux analysis in G595R patient A.II.3 was assessed in naïve enriched human CD56^{Dim} NK cells after crosslinking with NKG2D and 2B4. Open and closed red circles represent two unique blood samples acquired one year apart. (E) Western blot analysis for PLCG2 protein expression in HEK293T cells transfected with wild type or mutant FLAG-tagged PLCG2. EV, empty vector. (F) Phosphorylation of FLAG-tagged wildtype or mutant PLCG2 after 15 minutes of pervanadate stimulation in 293T cells is quantified using phospho-flow cytometry. EV, empty vector. Except where limited by patient sample availability (B.II.4 in A and B), all data is representative of two or more independent experiments.

FIG. 3A-FIG. 3E is a series of graphs and images showing PLCG2 haploinsufficiency alters cytotoxic granule mobility, NK cell maturation, and the adaptive NK cell response. (A) Representative immunofluorescence of cytotoxic granule microscopy upon K562 target conjugation in healthy versus patient A.II.3 NK cells. (B) Quantification of microtubule organizing center (MTOC) to granule distance (MGD), MTOC to synapse distance (MSD), and synaptic actin accumulation in healthy control (HC) versus patient A.II.3. *P<0.001, Mann-Whitney U Test. (C) CD107 Degranulation against K562 target cells is quantified by CyTOF after 1:1 incubation with healthy control or patient A.II.3 PBMCs for 6 hours. (D) viSNE on NK cells (CD3-CD56+) overlaid with maturity subpopulations identified by traditional bivariategating (top) with density visualized by contour (bottom) in both healthy control (HC) and patient A.II.3. Stage 1, NKG2A−CD57—, stage 2, NKG2A+CD57−, stage 3, NKG2A+CD57+, stage 4, NKG2A−CD57+. tSNE, t-distributed stochastic neighbor embedding. (E) Similar graphical representation as in (D) is shown for the adaptive NK cell response marker NKG2C. All data is representative of two or more independent experiments using two patient blood samples drawn more than 1 year apart. All error bars represent standard deviation from the mean.

FIG. 4A-FIG. 4E is a series of graphs showing heterozygous PIcg2 mice phenocopy human PLCG2 haploinsufficiency. Analysis of mouse immune cell subpopulations in the bone marrow (A), spleen and peripheral blood (B) of PIcg2 wildtype (+/+), heterozygous (+/−), and homozygous (−/−) littermates using flow cytometry and displayed using viSNE clustering as in FIG. 3D. Color key for cell types identified by traditional bivariate is located beneath each subfigure. tSNE, t-distributed stochastic neighbor embedding. (C) Analysis of splenic murine NK cell maturity in wild type littermate control versus heterozygous PIcg2 mice using CD27 and CD11b expression. DP, double positive. SP, single positive. Example bivariate gating of murine NK cell maturation is shown. Each point represents a unique biologic replicate. * P<0.05, Mann Whitney U Test. (D) Indo-1 calcium flux analysis of littermate PIcg2+/+, +/− and −/− mice is displayed after crosslinking with anti-IgM antibody (B cells gated from whole splenocytes) or anti-NK.1.1 antibody (NK cells enriched from spleen). (E) NK cell killing against YAC-1 and RMA-S target cells was analyzed in littermate wild type control versus heterozygous PIcg2 mice using enriched splenic NK cells at NK to target ratios of 1:10, 1:1 and 10:1. Pairwise comparisons at each time point performed using t-test, * P<0.05, after test for normality. All data is representative of two or more independent experiments. All error bars represent standard deviation from the mean.

FIG. 5A-FIG. 5C is a series of graphs showing PIcg2 G595R and L183F CRISPR mice demonstrate normal B cell development and perturbed NK cell function. (A) Analysis of NK cells, B cells, and memory B cells, in the peripheral blood from G595R knock-in, L183F knock-in or wild-type littermate controls using flow cytometry. Data points represent unique biological replicates. (B) Indo-1 calcium flux analysis G595R knock-in, L183F knock-in or wild-type littermate controls is displayed after crosslinking with anti-NK.1.1 antibody (NK cells enriched from spleen). (C) NK cell killing against YAC-1 target cells was analyzed in G595R knock-in, L183F knock-in or wild-type littermate controls at a NK to target ratio of 10:1. Pairwise comparisons performed using t-test, *P <0.05, after test for normality. Data points represent technical replicates. All data is representative of two or more independent experiments. All error bars represent standard deviation from the mean.

FIG. 6A-FIG. 6D is a series of graphs showing analysis of NK cells and B cells in PLCG2 haploinsufficiency. (A) Mass cytometry was performed to quantify total (CD3-CD56+), CD56^{Bright} and CD56^{Dim} NK cells in the peripheral blood of HC (healthy controls), G595R patients A.I.2, A.II.2, A.II.3 and L183F patient B.II.4. Internal reference ranges are shown as visualized by dashed lines. NK cell reference range, 2.8% to 15.5%. Each patient data point represents a unique biological replicate from a different blood draw. (B) B cells (HLADR+CD19+) are quantified and displayed as a percentage of PBMCs. Memory formation and class-switching is assessed by quantification of CD27+ IgM− cells within the B cell compartment. Normal B cell reference range, 6.2% to 20.2%. Each patient data point represents a unique biological replicate from a different blood draw. (C) ELISA quantification of serum IgG and IgM obtained from HC versus patients A.I.2,A.II.2 and A.II.3 (pooled above). IgG 487-1,327 mg/dL and IgM 37-286 mg/dL. Error bars represent standard deviation from the mean. (D) Indo-1 analysis of calcium flux in primary B cells (gated CD19+ from peripheral blood mononuclear cells) after crosslinking with anti-IgM.

FIG. 7A-FIG. 7B is a series of graphs showing analysis of T cells and myeloid cells in PLCG2 haploinsufficiency. (A) Mass cytometry was performed to quantify total T cells (CD3+) and T-follicular helper cells (TFH, CD3+ CD4+ PD-1+ CCR6−) in the peripheral blood of HC (healthy controls), G595R patients A.I.2, A.ll.2, A.II.3 and L183F patient B.ll.4. Internal reference ranges are shown as visualized by dashed lines. Each patient data point represents a unique biological replicate from a different blood draw. Error bars represent standard deviation. Distribution of polarized TFH cells is graphically displayed from each patient and two HC. TFH-1 gated from CXCR3+ CCR6− TFH cells, TFH-2 gated from CXCR3− CCR6− TFH cells, TFH-17 gated from CXCR3− CCR6+ TFH cells. (B) Myeloid lineage cells are quantified and displayed as a percentage of PBMCs. Normal reference ranges for all myeloid cells, 4.1% to 26.3%; classical monocytes, 1.91% to 16.4%; non-classical monocytes, 0.1% to 1.4%; pDC, 0.1% to 1.1%; mDC, 0.4% to 3.8%. Each patient data point represents a unique biological replicate from a different blood draw. Error bars represent standard deviation from the mean.

FIG. 8 is a series of graphs showing cyTOF analysis of CD56^{DIM} NK cell signaling. PBMCs were stimulated with 50ng/mL IL-12, 500U/mL IL-2, 500U/mL IFNα, 500 ng/mL LPS and 1 ug anti-mouse crosslinking antibody for CD16/CD3/IgM per 10⁶ cells for 0, 3 or 15 minutes before fixation. Analysis of signaling pathways was performed using mass cytometry to measure phosphoprotein levels after stimulation in CD56^{Dim} NK cells (gated as CD3− CD56+ CD16+). Values normalized to time 0 using an arcsinh transformation of the mean in three healthy controls (HC, black), two G595R patients (A.II.3 and A.I.2, red) and one L183F patient (B.II.4, blue). HC, healthy control. A.R.M., Arcsinh ratio of mean. Error bars represents standard deviation from the mean.

FIG. 9 is a graph showing total PLCG2 protein analysis by cell type. Total protein levels of PLCG2 were assessed in healthy controls (black) versus G595R patients (A.I.2, A.II.2 and A.II.3, red) by flow cytometry and intracellular staining. Cells were gated and the mean fluorescent intensity of PLCG2 was quantified. T cells, CD3+, B cells, CD3− CD19+; NK T Cells, CD3+ CD56+; CD56^{Bright} NK cells, CD3+ CD56++, CD16−; CD56^{Dim} NK Cells, CD3− CD56+ CD16+, Monocytes, HLADR+ CD14+ CD16−. Error bars represent standard deviation from the mean. Each data point represents a unique individual. Data is representative of two independent experiments.

FIG. 10A-FIG. 10C is a series of schematics and graphs showing conservation of PLCG2 and molecular dynamics analysis. (A) Conservation analysis of the G595 and L183 residues were generated using M-Coffee with ESPript secondary structure analysis. (B) Molecular dynamics analysis was performed to identify principal differences between the wild-type and G595R nSH2 domain of PLCG2 to understand potential structural effects from the G595R mutation. Molecular dynamics simulations of both sequences were ran and a shared state space Markov state model (MSM) was built for each sequence. One of the outputs of each MSM is the equilibrium probability that each state in the shared state space is occupied by each sequence. This allows direct comparison of each microstate's energetic favorability between sequences. Because there are more than two thousand microstates in this model, differences were systematically identified by computing the all-to-all Ca-Ca distances for each microstate, weighting each state by its population in the wild-type and G595R sequences, and comparing the means of these two distributions. The two distances that the highest positive (i.e., wild-type favored) and negative (mutant-favored) mean differences were chosen and are displayed (left). These two distances were the P570-G594 a-carbon distance and the

L605-G594 a-carbon distance. The difference in their joint distributions across all microstates is plotted as a 2-dimensional histogram (right). This analysis shows a clear change in the preference for adopting the P570-near, L605-far conformation in the mutant sequence and P570-far, L605-near conformation in the wild-type sequence. Green indicates site of mutation. Red, mutant preferred state. Blue, wild-type referred state. Bars identify key Ca-Ca distances. (C) APBS33 generated electrostatics and surface map of baseline versus G595R MD simulated structures. Red arrow depicts phosphotyrosine binding site.

FIG. 11A-FIG. 11D is a series of graphs showing additional patient calcium flux assays. (A) Indo-1 calcium flux analysis in G595R patient A.II.3 was assessed in CD56_{Bright} NK cells (CD3−CD56++CD16−) from naive enriched human NK cells after crosslinking with NKG2D and 2B4. Red circles (filled and empty), patient A.ll.3, black filled circles, HC (healthy control); black empty circles, isotype control. (B) Indo-1 calcium flux analysis in G595R patients A.II.3 and A.I.2 was assessed in CD56_{Dim} NK cells (CD3−CD56++CD16−) after crosslinking with CD16. Red circles (filled), patient A.II.3, red circles (empty), patient A.I.2, black filled circles, HC (healthy control); black empty circles, isotype control. (C) K562 killing assay using co-culture with expanded NK cells at E:T ratios of 1:4, 1:2 and 1:1 for 4 hours before assessment of K562 cytotoxicity. NK cells were expanded as previously described. Briefly, 10⁶ PBMCs from patient B.ll.4 or healthy control were co-incubated with 10⁶ irradiated (100Gy) K562-mbIL15-41bbl for 7 days. After 7 days, cells were removed and assessed for purity. T cells (CD3+) were present at less than <1%. 100 U/mL of recombinant IL-2 was added to the culture and incubated for 7 more days, with partial media exchange every 2 days. After 14 days total, NK cells expanded 10 to 15-fold with >95% purity (CD56+CD3−) and were then used for cytotoxicity and calcium flux assays. (D) Indo-1 calcium flux analysis in G595R patients A.II.3 and A.I.2 and L183F patient B.II.4 was assessed in expanded NK cells (as in (C)) after crosslinking with NKG2D and NKp44. Red circles (filled), patient A.ll.3, red circles (empty), patient A.I.2, black filled circles, HC (healthy control); blue circles, patient B.II.4.

FIG. 12A-FIG. 12B is a series of schematics showing mouse and human gating schemes for flow cytometry and mass cytometry. Gating strategies used in both flow cytometry and mass cytometry experiments for human samples (A) and murine samples (B).

FIG. 13A-FIG. 13B is a series of graphs showing PBMC percentages in JDM patients and healthy controls. Open circles denote treatment-naive patients (n=17). Filled squares denote healthy controls (n=17). (A) Percentage of PBMC population in treatment-naive patients and controls for higher frequency (left panel) and lower frequency (right panel) immune cell types (1-way ANOVA: F=7.429, P<0.001; naive B cells: t=7.459, P<0.05; naive CD4+T cells: t=6.561, P<0.05;NK cells: t=4.415, P<0.05). (B) Percentage of PBMC populations in paired treatment-naive and clinically inactive disease patient samples for higher frequency (left panel) and lower frequency (right panel) immune cell types (1-way ANOVA: F=36.15, P<0.005; naive B cells: t=6.986, P<0.05, and n=11 paired patient samples). x′s denote patients after achieving clinically inactive disease (n=11). Error bars represent the mean ±SEM. *P <0.05 after appropriate multiple hypothesis correction.

FIG. 14A-FIG. 14D is a series of graphs showing signaling molecules in several immune cell subsets were stratifying between treatment-naive JDM patients and healthy controls for unstimulated as well as 3- and 15-minute-stimulated samples. Citrus was used to identify stratifying clusters (n=17 treatment-naive patients, n=17 matched controls in all subpanels). (A) Heatmap of arcsinh median intensity for surface markers used for Citrus clustering for stratifying clusters detected by Citrus at all time points (cluster numbers are denoted on the right side of the figure). Unst: unstimulated. (B) Heatmap of arcsinh-transformed median signaling molecule intensity of stratifying signaling molecules in the respective clusters for all time points retained by LASSO feature selection with the minimum cross-validation error as the threshold. Rows correspond to signaling features, and columns correspond to samples, with red denoting treatment-naive patients and black representing healthy controls. Pt: patient; CM: classical monocytes; CD4T: CD4+ T cells; CD8T: CD8+ T cells; NCM: nonclassical monocytes. (C) PLS-DA scores plot for classification of treatment-naive patients and controls developed using LASSO-selected features from Citrus in (B). Red points correspond to treatment-naive patients and black to controls. (D) PLS-DA loadings plot (depiction of relationship of variables to one another in dimensionally reduced variable space) for classification of treatment-naive patients and controls developed using LASSO-selected features from Citrus in (B).

FIG. 15A-FIG. 15D is a series of graphs showing treatment-naive JDM patient NK cells hypophosphorylate PLCG2 and MAPKAPK2 but not Syk/ZAP70 and ltk/Btk in comparison with controls over stimulation time course (tested with 2-way Welch's t tests with Benjamini-Hochberg multiple hypothesis correction for 12 tests). Open circles denote treatment-naive patients (n=17). Filled squares denote healthy controls (n=17). Data are displayed as the arcsinh ratio of the median intensity of the sample normalized to the run control. (A) NK cell PLCG2 (0-min P=6.88×10⁻⁶, 3-min P=1.62×10⁻⁶, and 15-min P=1.47×10⁻⁶) phosphorylation differs between treatment-naive JDM patients and controls. (B) NK cell Syk/ZAP70 and (C) ltk/Btk phosphorylation are not different between treatment-naive JDM patients and controls. (D) MAPKAPK2 (0-min P=0.003, 3-min P=0.002, and 15-min P=0.0008) phosphorylation differs over the time course between 17 treatment-naive JDM patients and 17 controls. Error bars represent the mean ±SEM. *P<0.05.

FIG. 16A-FIG. 16E is a series of graphs showing evaluation of total PLCG2, SHIP1, and CD16 levels. Open circles denote treatment-naive patients. Filled squares denote healthy controls. (A) Total PLCG2 protein levels determined with flow cytometry (n=3 treatment-naive patients, n=3 controls; 2-way Welch's t test: t=1.662, df=4, P=0.1719). (B) Total SHIP1 protein levels determined with flow cytometry (n=3 treatment-naive patients, n=3 controls; 2-way Welch's t test: t=3.701, df=4, P=0.0208). (C) Arcsinh transformation of CD16 in treatment-naive JDM patient (open circles) and control (filled squares) NK cells assessed with mass cytometry (1-way Welch's t test: t=1.968, df=25, P=0.0301, n=17 patients, and n=17 controls). (D) Correlation of integrated p-PLCG2 time course versus arcsinh MFI CD16 for patients and controls (treatment-naive patients: y=−2.78+0.80x, r=0.66, P=0.0039, and n=17; patients with clinically inactive disease: y=−0.44+0.568x, r=0.38, P=0.254, and n=11; controls: y=2.12+0.15x, r=0.17, P=0.51, n=17). Tmt:

treatment; Dis: disease. (E) Arcsinh transformation of CD16 in NK cells from treatment-naive JDM patients (open circles) and paired JDM patients with clinically inactive disease (x′s) (1-way paired Welch's t test: t=1.343, df=10, P=0.209, n=11 treatment-naive patients, and n=11 patients with clinically inactive disease). Error bars represent the mean ±SEM. *P<0.05.

FIG. 17A-FIG. 17C is a series of graphs showing enriched NK cells from treatment-naive JDM patients exhibit decreased Ca²⁺ flux compared with NK cells from healthy controls upon stimulation by 2B4 and NKG2D receptor cross-linking. (A) Calcium flux in treatment-naive patient and control NK cells (n=2 treatment-naive patients, n=1 matched control). The cell surface expression of 2B4 (B) and NKG2D (C) was similar in the treatment-naive JDM patients and controls.

FIG. 18A-FIG. 18B is a series of schematics showing gating schemes for (A) live singlet lymphocytes and (B) immune cell subsets.

FIG. 19A-FIG. 19C is a series of graphs showing citrus error plots for LASSO classification models between treatment naïve JDM patients and controls for (A) unstimulated samples, (B) 3 minute stimulated samples, and (C) 15 minute stimulated samples.

FIG. 20A-FIG. 20C is a series of graphs showing assessment of p-PLCG2 and p-MAPKAPK2 in JDM patients with clinical inactive disease and in relationship to MSA autoantibody status. Data are displayed as the arcsinh ratio of the median intensity of the sample normalized to the run control. (A) PLCG2 phosphorylation time course in NK cells from JDM treatment-naïve patients (n=17), JDM patients with clinical inactive disease (n=11), and healthy controls (n=17). PLCG2 phosphorylation in NK cells from JDM patients with clinical inactive disease is intermediate between that observed in NK cells from JDM treatment-naïve patients and controls. (B), MAPKAPK2 phosphorylation time course in NK cells for treatment-naïve patients (n=17), patients with clinical inactive disease (n=11), and controls (n=17). MAPKAPK2 phsophorylation in NK cells from JDM patients with clinical inactive disease is intermediate between that observed in NK cells from JDM treatment-naïve patients and controls, (C) PLCG2 phosphorylation in NK cells from treatment-naïve JDM patients (n=17) is compared with two subsets of treatment-naïve JDM patients with p155/140 autoantibodies (n=9) or no MAS antibodies (n=3). Asterisk denote significance between treatment-naïve JDM patients and controls.

FIG. 21 is a graph showing NK cell frequency correlates with PLCG2 phosphorylation intensity in treatment-naïve JDM patient NK cells. Correlation of integrated p-PLCG2 time course versus NK cell percentage for JDM patients and controls (treatment naïve patients: y=−2.39+0.42x, R=0.55, p=0.0235, n=17; clinical inactive disease patients: y=1.05−0.039x, R=−0.051, p=0.88, n=11; controls: y=2.12+0.059x, R=0.35, p=0.167, n=17).

FIG. 22A-FIG. 22B is a series of graphs showing treatment-naïve JDM patient and control NK cells differ in activation and proliferation, as assessed by CD69 and Ki67 expression levels. Data are displayed as the arcsinh ratio of the median intensity of the sample normalized to the run control. (A) Signal intensity of CD69 in treatment naïve JDM patient (n=17) and control (n=17) NK cells (t=3.327, df=32, p=0.0022), (B) Signal intensity of Ki-67 in treatment naïve JDM patient (n=17) and control (n=17) NK cells (t=5.463, df=32, p<0.0001).

FIG. 23A-FIG. 23D is a series of graphs showing NK cell phosphorylation time courses with patient 7 mapped separately from n=16 other treatment-naïve patients and n=17 matched controls. Data are displayed as the arcsinh ratio of the median intensity of the sample to the run control. (A) NK cells p-PLCG2 phosphorylation, (B) NK cell Syk/ZAP70 phosphorylation, (C) ltk/Btk phosphorylation, and, (D) MAPKAPK2 phosphorylation.

FIG. 24 shows phosphorylation time courses for manually gated immune cell populations similar to those retained by LASSO in Citrus (n=17 patients, n=17 controls). It should be noted that in contrast to the upregulation of PLCG2 phosphorylation identified in the stratifying clusters of CD4 and CD8 T cells in the Citrus analysis (see e.g., FIG. 15D), the phosphorylation of PLCG2 in bulk populations (manually gated) of CD4 and CD8 T cells was decreased in treatment-naïve JDM patients compared to controls (see e.g., FIG. 24). The significance of this observation in regard to JDM is unclear since T cells primarily utilize PLCG2, and no defects in T cell function were observed in mice in which PLCG2 was knocked out. Open circles with solid lines indicate patients. Closed squares with dotted lines indicate controls. Asterisk denotes a statistically significant differences between treatment-naïve patients and controls at each timepoint.

### DETAILED DESCRIPTION OF THE INVENTION

The present disclosure is based, at least in part, on the discovery that subjects suffering from juvenile dermatomyositis (JDM) can be distinguished from controls by phospholipase C gamma-2 (PLCG2) hypophosphorylation, which results in decreased calcium flux in natural killer (NK) cells. The decreased calcium flux has functional consequences that affect NK-cell mediated cytotoxicity and suppression of inappropriate adaptive immune responses, which can result in autoimmunity, autoimmune disease, or inflammatory diseases.

As described herein, hypophosphorylation of PLCG2 in NK cells leads to decreased calcium flux which results in poor movement of the cytotoxic granules to the immune synapse where NK cells kill infected, transformed, or inappropriately activated cells. These defects in NK cell activation appear to be attenuated with achievement of clinical remission and recrudescence prior to flares. Furthermore, evidence that PLCG2 hypophosphorylation is attenuated by cytokines is presented. For example, any cytokine capable of restoring normal function and normal calcium flux in a dysfunctional natural killer (NK) cell can be used.

NK cells are innate immune cells that rapidly respond to viral infections and potentially play a role in the suppression of inappropriate adaptive immune responses. NK cells perform these functions by making immunomodulatory cytokines (e.g., IFN-y) and by killing infected, transformed, or inappropriately activated cells. PLCG2 is a critical enzyme in NK cell activation. Phosphorylation of PLCG2 results in calcium flux within NK cells with subsequent cytolytic granule movement and localization to the immune synapse, which facilitates targeted NK cell-mediated cytotoxicity.

As described herein, mass cytometry (CyTOF) was recently employed to investigate the phosphorylation status of a broad panel of signaling proteins in different immune cell subsets in treatment-naïve juvenile dermatomyositis (JDM) patients and healthy age-matched controls (see e.g., Example 2). Using this approach, it was identified that NK cell PLCG2 hypophosphorylation was the primary signaling abnormality distinguishing treatment-naïve JDM patients from healthy controls. No differences were detected in upstream phosphorylation in Syk and ITK or in total PLCG2 protein levels in NK cells. Interestingly, the hypophosphorylation of PLCG2 was attenuated in samples from JDM patients who had achieved clinically inactive disease, further supporting a role for NK cell PLCG2 in JDM. Furthermore, it was demonstrated that suppressed PLCG2 phosphorylation in treatment-naïve JDM patient NK cells resulted in decreased calcium flux, suggesting that this signaling defect has functional consequences.

Described herein is additional evidence showing the ability of cytokines such as IL-2 to substantially normalize calcium flux in the context of PLCG2 haploinsufficiency in patients with unusually recurrent and or severe herpesvirus infection (see e.g., Example 1 for NK cell defect rescue data). In these patients, the hypophosphorylation of PLCG2 results in decreased calcium flux and subsequent poor NK cell granule movement and localization to the immune synapse and subsequent NK cell cytotoxicity. Also described herein is the discovery that IL-2 substantially normalizes the decreased calcium flux in these patients, suggesting that cytokine therapy (e.g., IL-2 or IL-15) may correct this early signaling defect in JDM and provide a potential therapeutic intervention in mitigating the impact of this autoimmune disease in children and potentially in adults with DM.

NATURAL KILLER (NK) CELLS

The present disclosure provides for methods of modulating natural killer (NK) cells and NK cell function. There is accumulating evidence that human NK cells play an immunoregulatory role and that NK cell dysfunction may contribute to the onset of human autoimmunity. NK cells are innate lymphocytes (defined as CD3-CD56+) with germline-encoded receptors that play a critical role in antiviral defense and tumor surveillance, and potentially play a role in the suppression of inappropriate adaptive immune responses. NK cells perform these critical functions by secreting immunomodulatory cytokines and releasing cytotoxic granules to lyse infected, transformed, or inappropriately activated cells. The movement of cytotoxic granules within NK cells is regulated by the phosphorylation of phospholipase Cγ2 (PLCG2) and subsequent generation of calcium flux. As described in Example 1 and Example 2, PLCG2 encodes a signaling protein in NK cell and B cell receptor-mediated signaling, and PLCG2 hypophosphorylation can lead to defects in NK cell function (e.g., functional natural killer deficiency (NKD)).

In some embodiments, modulating NK cell function refers to modulation of NK calcium flux, granule movement, or cytotoxicity. Modulation of NK cell function can be determined by measuring NK cell killing and CD107 degranulation after incubation with K562 target cells in a sample and comparing to a wild-type or healthy control sample. As another example, flow cytometry-based calcium flux assays can be performed to assess NK calcium flux in a sample and compared to a wild-type or healthy control sample.

PLCG2 phosphorylation results in a conformational change in PLCG2, facilitating the hydrolysis of the membrane phospholipid phosphatidylinositol 4,5-bisphosphate to inositol triphosphate (IP3) and diacylglycerol. IP3 subsequently binds to its receptor on the endoplasmic reticulum and releases cellular stores of calcium. Decreased calcium flux is associated with altered cytotoxic granule movement and localization to the immune synapse, resulting in poor NK cell-mediated killing. Therefore, PLCG2 hypophosphorylation and decreased calcium flux results in NK cells with decreased NK cell cytotoxicity. As an example, modulation of NK cell cytotoxicity can be determined by exposing tumor target cells to NK cells in a sample and quantifying target cell death using flow cytometry, and comparing to a wild-type or healthy control sample.

Other tests as described herein also use control samples. For example, a control sample or a reference sample as described herein can be a sample from a subject with clinically inactive disease or from a healthy subject. A reference value can be used in place of a control or reference sample, which was previously obtained from a subject at initial presentation, a healthy subject, a group of subjects with initial presentation or healthy subjects. As another example, a control sample or a reference sample can also be a sample with a known amount of a detectable compound or a spiked sample. As another example, a control can be any control for determining modulation of NK cell function known in the art.

There is accumulating evidence that NK cells play a role in the initiation of autoimmunity. It is presently believed that this is due to a suppression of NK cell functional responses (particularly killing) resulting in the release of an important brake on autoimmune T cell responses. NK cells have been implicated in human autoimmunity and regulation of T cells (the primary effector cell driving autoimmunity). There is also evidence that decreased NK cell killing may be implicated in JDM and DM. Furthermore, suppressed NK cell killing is implicated in Multiple Sclerosis that preceded MS flares in patients not on medications. As such, the novel regulation of PLCG2 to increase NK cell cytotoxicity, as described herein, can be used as a therapeutic for these autoimmune and inflammatory conditions.

NK cell dysfunction can be determined by any method known in the art. Here, it was shown that dysfunctional NK cells exhibit dysregulated PLCG2 signaling, PLCG2 haploinsufficiency, or PLCG2 hypophospohorylation. As another example, dysfunctional NK cells can have dominant negative or gain of function mutations.

PLCG2 Hypophosphorylation-Associated Diseases, Disorders, or Conditions

The present disclosure provides for methods of treatment of subjects having or at risk of having a PLCG2 hypophosphorylation-associated disease, disorder, or condition. A PLCG2 hypophosphorylation-associated disease, disorder, or condition is any disease, disorder, or condition that is associated with or results from reduced phosphorylation of the PLCG2 protein (PLCG2 hypophosphorylation), when compared to a healthy control.

Autoimmune diseases, disorders, or conditions

A PLCG2 hypophosphorylation-associated disease, disorder, or condition can be an autoimmune disease, disorder, or condition. The present disclosure provides for treatment of autoimmune diseases, disorders, or conditions. It is presently believed that decreased NK cell killing is associated with autoimmune diseases and autoimmunity. As described herein, hypophosphorylation of PLCG2 results in poor NK cell killing in juvenile dermatomyositis (JDM). As such, it is presently thought that hypophosphorylation of PLCG2 is likely to be a mechanism of poor NK cell killing in other autoimmune diseases, disorder, or conditions.

In some embodiments, a PLCG2 hypophosphorylation-associated disease, disorder, or condition can be an autoimmune disease, disorder, or condition. For example, an autoimmune disease, disorder, or condition can be Achalasia; Addison's disease; Adult Still's disease; Agammaglobulinemia; Alopecia areata; Amyloidosis; Ankylosing spondylitis; Anti-GBM/Anti-TBM nephritis; Antiphospholipid syndrome; Autoimmune angioedema; Autoimmune dysautonomia; Autoimmune encephalomyelitis; Autoimmune hepatitis; Autoimmune inner ear disease (Al ED); Autoimmune myocarditis; Autoimmune oophoritis; Autoimmune orchitis; Autoimmune pancreatitis; Autoimmune retinopathy; Autoimmune urticaria; Axonal & neuronal neuropathy (AMAN); Baló disease; Behcet's disease; Benign mucosal pemphigoid; Bullous pemphigoid; Castleman disease (CD); Celiac disease; Chagas disease; Chronic inflammatory demyelinating polyneuropathy (CIDP); Chronic recurrent multifocal osteomyelitis (CRMO); Churg-Strauss Syndrome (CSS) or Eosinophilic Granulomatosis (EGPA); Cicatricial pemphigoid; Cogan's syndrome; Cold agglutinin disease;

Congenital heart block; Coxsackie myocarditis; CREST syndrome; Crohn's disease; Dermatitis herpetiformis; Dermatomyositis (DM); Devic's disease (neuromyelitis optica); Discoid lupus; Dressler's syndrome; Endometriosis; Eosinophilic esophagitis (EoE); Eosinophilic fasciitis; Erythema nodosum; Essential mixed cryoglobulinemia; Evans syndrome; Fibromyalgia; Fibrosing alveolitis; Giant cell arteritis (temporal arteritis); Giant cell myocarditis; Glomerulonephritis; Goodpasture's syndrome; Granulomatosis with Polyangiitis; Graves' disease; Guillain-Barre syndrome; Hashimoto's thyroiditis; Hemolytic anemia; Henoch-Schonlein purpura (HSP); Herpes gestationis or pemphigoid gestationis (PG); Hidradenitis Suppurativa (HS) (Acne Inverse);

Hypogammalglobulinemia; IgA Nephropathy; IgG4-related sclerosing disease; Immune thrombocytopenic purpura (ITP); Inclusion body myositis (IBM); Interstitial cystitis (IC); juvenile dermatomyositis (JDM), Juvenile arthritis; Juvenile diabetes (Type 1 diabetes); Juvenile myositis (JM); Kawasaki disease; Lambert-Eaton syndrome; Leukocytoclastic vasculitis; Lichen planus; Lichen sclerosus; Ligneous conjunctivitis; Linear IgA disease (LAD); Lupus; Lyme disease chronic; Meniere's disease; Microscopic polyangiitis (MPA); Mixed connective tissue disease (MCTD), Mooren's ulcer; Mucha-Habermann disease; Multifocal Motor Neuropathy (MMN) or MMNCB; Multiple sclerosis; Myasthenia gravis; Myositis; Narcolepsy; Neonatal Lupus; Neuromyelitis optica; Neutropenia; Ocular cicatricial pemphigoid; Optic neuritis; Palindromic rheumatism (PR); PANDAS; Paraneoplastic cerebellar degeneration (POD); Paroxysmal nocturnal hemoglobinuria (PNH); Parry Romberg syndrome; Pars planitis (peripheral uveitis); Parsonage-Turner syndrome; Pemphigus; Peripheral neuropathy; Perivenous encephalomyelitis; Pernicious anemia (PA); POEMS syndrome; Polyarteritis nodosa; Polyglandular syndromes type I, II, Ill; Polymyalgia rheumatica; Polymyositis; Postmyocardial infarction syndrome; Postpericardiotomy syndrome; Primary biliary cirrhosis; Primary sclerosing cholangitis; Progesterone dermatitis; Psoriasis; Psoriatic arthritis; Pure red cell aplasia (PRCA); Pyoderma gangrenosum; Raynaud's phenomenon; Reactive Arthritis; Reflex sympathetic dystrophy; Relapsing polychondritis; Restless legs syndrome (RLS); Retroperitoneal fibrosis; Rheumatic fever; Rheumatoid arthritis; Sarcoidosis; Schmidt syndrome; Scleritis; Scleroderma; Sjögren's syndrome; Sperm & testicular autoimmunity; Stiff person syndrome (SPS); Subacute bacterial endocarditis (SBE); Susac's syndrome; Sympathetic ophthalmia (SO); Takayasu's arteritis; Temporal arteritis/Giant cell arteritis; Thrombocytopenic purpura (TTP); Tolosa-Hunt syndrome (THS); Transverse myelitis; Type 1 diabetes; Ulcerative colitis (UC), Undifferentiated connective tissue disease (UCTD), Uveitis; Vasculitis; Vitiligo; or Vogt-Koyanagi-Harada Disease.

Infectious Diseases

As described herein PLCG2 dysfunction can increase susceptibility to infectious diseases such as viral and bacterial infections.

As such, a PLCG2 hypophosphorylation-associated disease, disorder, or condition can be Acute Flaccid Myelitis (AFM); Anaplasmosis; Anthrax; Babesiosis; Botulism; Brucellosis; Campylobacteriosis, Carbapenem-resistant Infection (CRE/CRPA), Chancroid, Chikungunya Virus Infection (Chikungunya), Chlamydia, Ciguatera (Harmful Algae Blooms (HABs)); Clostridium Difficile Infection; Clostridium Perfringens (Epsilon Toxin); Coccidioidomycosis fungal infection (Valley fever); Creutzfeldt-Jacob Disease, transmissible spongiform encephalopathy (CJD); Cryptosporidiosis (Crypto); Cyclosporiasis; Dengue, 1,2,3,4 (Dengue Fever); Diphtheria; E. coli infection; Shiga toxin-producing (STEC); Eastern Equine Encephalitis (EEE); Ebola Hemorrhagic Fever (Ebola); Ehrlichiosis; Encephalitis, Arboviral or parainfectious; Enterovirus Infection; Non-Polio (Non-Polio Enterovirus); D68 (EV-D68); Giardiasis (Giardia); Glanders; Gonococcal Infection (Gonorrhea); Granuloma inguinale; Haemophilus Influenza disease, Type B (Hib or H-flu); Hantavirus Pulmonary Syndrome (HPS); Hemolytic Uremic Syndrome (HUS); Hepatitis A (Hep A); Hepatitis B (Hep B); Hepatitis C (Hep C); Hepatitis D (Hep D); Hepatitis E (Hep E); Herpesvirus; Herpes Zoster, zoster VZV (Shingles); Histoplasmosis infection (Histoplasmosis); Human Immunodeficiency Virus/AIDS (HIV/AIDS); Human Papillomavirus (HPV); Influenza (Flu); Legionellosis (Legionnaires Disease); Leprosy (Hansens Disease); Leptospirosis; Listeriosis (Listeria); Lyme Disease; Lymphogranuloma venereum infection (LGV); Malaria; Measles; Melioidosis; Meningitis, Viral (Meningitis, viral); Meningococcal Disease (e.g., Meningitis, bacterial); Middle East Respiratory Syndrome Coronavirus (MERS-CoV); Mumps; Norovirus; Paralytic Shellfish Poisoning (Paralytic Shellfish Poisoning, Ciguatera); Pediculosis (Lice, Head and Body Lice); Pelvic Inflammatory Disease (PID); Pertussis (Whooping Cough); Plague (e.g., Bubonic, Septicemic, Pneumonic); Pneumococcal Disease (Pneumonia); Poliomyelitis (Polio); Powassan; Psittacosis (Parrot Fever); Pustular Rash diseases (Small pox, monkeypox, cowpox); Q-Fever; Rabies; Ricin Poisoning; Rickettsiosis (Rocky Mountain Spotted Fever); Rubella, Including congenital (German Measles); Salmonellosis gastroenteritis (Salmonella); Scabies Infestation (Scabies); Scombroid; Septic Shock (Sepsis); Severe Acute Respiratory Syndrome (SARS); Shigellosis gastroenteritis (Shigella); Smallpox; Staphyloccal Infection; Methicillin-resistant Staphylococcus aureus (MRSA); Staphylococcal Food

Poisoning, Enterotoxin - B Poisoning (Staph Food Poisoning); Staphylococcal Infection, Vancomycin Intermediate (VISA); Vancomycin Resistant Staphylococcus aureus (VRSA); Streptococcal Disease, Group A (invasive) (Strep A (invasive)); Streptococcal Disease, Group B (Strep-B); Streptococcal Toxic-Shock Syndrome (STSS); Toxic Shock syndrome (TSS); Syphilis; Tetanus Infection; Trichomoniasis (Trichomonas infection); Trichonosis Infection (Trichinosis); Tuberculosis (TB); Tuberculosis (Latent) (LTBI); Tularemia (Rabbit fever); Typhoid Fever, Group D; Typhus; Vaginosis, bacterial, fungal (e.g., Yeast Infection); Varicella (Chickenpox); Vibrio cholerae (Cholera); Vibriosis (Vibrio); Viral Hemorrhagic Fever (Ebola, Lassa, Marburg); West Nile Virus; Yellow Fever; Yersenia (Yersinia); or Zika Virus Infection (Zika).

Viral Infections

A PLCG2 hypophosphorylation-associated disease, disorder, or condition can be a viral infection. In some embodiments, a PLCG2 hypophosphorylation-associated disease, disorder, or condition can present as susceptibility to unusually severe or recurrent viral infection. For example, a viral infection can be caused by Adenovirus, Herpes simplex (type 1 or type 2), Varicella-zoster virus, Epstein-Barr virus, Human cytomegalovirus, Human herpesvirus, Human papillomavirus, Smallpox, Hepatitis B virus, Parvovirus B19, Human astrovirus, Norwalk virus, Coxsackievirus, Hepatitis A virus, Poliovirus, Rhinovirus, Severe acute respiratory syndrome virus, Hepatitis C virus, Yellow fever virus, Dengue virus, West Nile virus, TBE virus, Rubella virus, Hepatitis E virus, Human immunodeficiency virus, Influenza virus, Lassa virus, Crimean-Congo hemorrhagic fever virus, Hantaan virus, Ebola virus, Marburg virus, Measles virus, Mumps virus, Parainfluenza virus, Respiratory syncytial virus, Rabies virus, Hepatitis D, Rotavirus, Orbivirus, Coltivirus, or Banna virus.

PLCG2 Haploinsufficiency

In some embodiments, a PLCG2 hypophosphorylation-associated disease, disorder, or condition can result from genetic haploinsufficiency. PLCG2 haploinsufficiency is a clinically and mechanistically distinct syndrome from previously reported PLCG2 mutations. Previously reported mutations, associated with PLAID and APLAID, are autosomal dominant manifestations of dominant-negative and gain-of-function mutations. PLCG2 haploinsufficiency results in clinical phenotypes distinct from PLAID/APLAID (e.g., B cell function remains intact in PLCG2 haploinsufficiency) and requires a different diagnostic and therapeutic approach. As described herein, heterozygous, loss-of-function mutations in PLCG2 in human patients results in haploinsufficiency, and these mutations result in impaired natural killer (NK) cell function and recurrent herpesvirus infections (see e.g., Example 1). For example, the heterozygous mutation in PLCG2 can be a G595R or a L183F mutation. As another example, haploinsufficiency can result in reduced or loss of PLCG2 function (e.g., loss-of-function), activity, or expression.

Inflammatory Diseases, Disorders, or Conditions

A PLCG2 hypophosphorylation-associated disease, disorder, or condition can be an inflammatory disease, disorder, or condition. For example, the inflammatory disease can be asthma, chronic peptic ulcer, tuberculosis, rheumatoid arthritis, periodontitis, ulcerative colitis, Crohn's disease, sinusitis, active hepatitissome cancers, rheumatoid arthritis (RA), atherosclerosis, periodontitis, hay fever, multiple sclerosis (MS), Ankylosing Spondylitis (AS); Antiphospholipid Antibody Syndrome (APS); Gout; Inflammatory Arthritis Center; Inflammatory Bowel Disease, Myositis; Rheumatoid Arthritis; Scleroderma; Sjogren's Syndrome; Systemic Lupus Erythematosus (SLE, Lupus); or Vasculitis.

As another example, a PLCG2 hypophosphorylation-associated disease, disorder, or condition can be an autoinflammatory disease. For example, the autoinflammatory disease can be Familial Mediterranean Fever (FMF); neonatal

Onset Multisystem Inflammatory Disease (NOMID); Tumor Necrosis Factor Receptor-Associated Periodic Syndrome (TRAPS); Deficiency of the Interleukin-1 Receptor Antagonist (DI RA); Behcet's Disease; or Chronic Atypical Neutrophilic Dermatosis with Lipodystrophy and Elevated Temperature (CANDLE).

PLCG2 Phosphorylation Modulating Agents

The present disclosure provides for administration of PLCG2 phosphorylation modulating agents comprising one or more cytokines. A PLCG2 phosphorylation modulating agent can be an agent that provide for a blockade of IFNα or IFNγ.

Immunologic context, such as the cytokine environment, may modulate cellular and clinical phenotypes resulting from PLCG2 hypophosphorylation. As described herein, PLCG2 hypophosphorylation can be attenuated by cytokines (see e.g., Example 1). For example, IL-15/IL-2 cytokine exposure was used to restore NK cell killing in patients with PLCG2 haploinsufficiency (see e.g., Example 1 and FIG. 11C). As described herein, any number of cytokines that activate NK cells, modulate calcium flux, or restore NK cell function may be used as PLCG2 phosphorylation modulating agents. For example, a cytokine can be IL-2, IL-15, IL-18, IL-12, or COLS.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in

United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0 ° C. and about 60 ° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, subcutaneous, intranasal, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time.

In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating or preventing a disease, disorder, or condition associated with PLCG2 hypophosphorylation in NK cells or in a subject comprising administration of a therapeutically effective amount of a PLCG2 phosphorylation modulating agent (e.g., comprising one or more cytokines), so as to modulate calcium flux or restore NK cell function.

Methods described herein are generally performed on a subject in need thereof. A subject in need of the therapeutic methods described herein can be a subject having, diagnosed with, suspected of having, or at risk for developing a disease, disorder, or condition associated with PLCG2 hypophosphorylation. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of a PLCG2 phosphorylation modulating agent is, for example, that amount that would cause the desired therapeutic effect in a subject while minimizing undesired side effects. In various embodiments, an effective amount of a PLCG2 phosphorylation modulating agent described herein can substantially inhibit a PLCG2 hypophosphorylation-associated disease, disorder, or condition, slow the progress of a PLCG2 hypophosphorylation-associated disease, disorder, or condition, or limit the development of a PLCG2 hypophosphorylation-associated disease, disorder, or condition.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of a PLCG2 phosphorylation modulating agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to modulate calcium flux or restore NK cell function.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a PLCG2 phosphorylation modulating agent can occur as a single event or over a time course of treatment. For example, a PLCG2 phosphorylation modulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease, disorder, or condition associated with PLCG2 hypophosphorylation (e.g., steroid, antiviral).

A PLCG2 phosphorylation modulating agent can be administered simultaneously or sequentially with another agent, such as an antibiotic, an anti-inflammatory, a steroid, an antiviral, or another agent. For example, a PLCG2 phosphorylation modulating agent can be administered simultaneously with another agent, such as an antibiotic, an anti-inflammatory, a steroid, or an antiviral. Simultaneous administration can occur through administration of separate compositions, each containing one or more of a PLCG2 phosphorylation modulating agent, an antibiotic, an anti-inflammatory, a steroid, an antiviral, or another agent. Simultaneous administration can occur through administration of one composition containing two or more of a PLCG2 phosphorylation modulating agent, an antibiotic, an anti-inflammatory, a steroid, an antiviral, or another agent. A PLCG2 phosphorylation modulating agent can be administered sequentially with an antibiotic, an anti-inflammatory, or another agent. For example, a PLCG2 phosphorylation modulating agent can be administered before or after administration of an antibiotic, an anti-inflammatory, a steroid, an antiviral, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Screening

Also provided are methods for screening for PLCG2 phosphorylation modulating agents using the methods as described herein.

The subject methods find use in the screening of a variety of different candidate molecules (e.g., potentially therapeutic candidate molecules).

Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001)

Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims.

Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Human PLCG2 Haplonsufficiency Results in Natural Killer Cell Immunodeficiency and Herpesvirus Susceptibility

This example describes the discovery of the first heterozygous, loss-of-function mutations in PLCG2 in human patients, and how these mutations result in impaired natural killer (NK) cell function and recurrent herpesvirus infections.

Abstract

Although most individuals effectively control herpesvirus infections, some suffer from unusually severe and/or recurrent infections requiring anti-viral prophylaxis. A subset of these patients possess defects in natural killer (NK) cells, innate lymphocytes which recognize and lyse herpesvirus-infected cells; however, the genetic etiology is rarely diagnosed. PLCG2 encodes a signaling protein in NK cell and B cell receptor-mediated signaling. Dominant-negative or gain-of-function mutations in PLCG2 cause cold urticaria, antibody deficiency, or autoinflammation. However, loss-of-function mutations and PLCG2 haploinsufficiency do not appear to have been previously reported in human disease. Using mass cytometry and whole-exome sequencing, novel heterozygous mutations in PLCG2 in two families with severe and/or recurrent herpesvirus infections were identified. In vitro studies demonstrated that these mutations were loss-of-function and resulted in impaired NK calcium flux, granule movement, and cytotoxicity. In contrast to dominant-negative or gain-of-function PLCG2 mutations, B cell function remained intact. PIcg2+/− mice, as well as targeted CRISPR knock-in mice, also displayed impaired NK cell function with preserved B cell function, phenocopying human PLCG2 haploinsufficiency.

Described herein are what appear to be the first known cases of PLCG2 haploinsufficiency, a clinically and mechanistically distinct syndrome from previously reported mutations. Therefore, these families represent a novel disease, highlighting a role for PLCG2 haploinsufficiency in herpesvirus-susceptible patients and expanding the spectrum of PLCG2-related disease.

Introduction

Nearly all individuals will encounter herpesviruses such as herpes simplex virus 1 (HSV1) or cytomegalovirus (CMV) in their lifetime. Although most will present with only limited recurrences, some patients will continue to have unusually severe and/or recurrent herpesvirus infections. A subset of these patients possess defects in natural killer (NK) cells, innate lymphocytes which recognize and lyse herpesvirus-infected cells. NK cell deficiency (NKD) can result from either aberrant NK cell development (classical NKD) or reduced NK cell function (functional NKD), typically evaluated by measuring NK cell killing and CD107 degranulation after incubation with K562 target cells. Despite these diagnostic tools, there is minimal understanding of the genetics underlying functional NKD, and most patients do not receive a definitive diagnosis. Described herein is a novel syndrome of functional NKD and herpesvirus susceptibility caused by heterozygous loss-of-function mutations in PLCG2.

NK cells recognize herpesvirus-infected cells using an array of germline-encoded activating receptors, such as CD16, NKG2D, and 2B4. Many activating receptors signal via a pathway involving Src and Syk kinases, LAT, and PLCG2-induced secondary messengers. PLCG2 (encoding phospholipase C-y2) is recruited to LAT, phosphorylated, and subsequently cleaves PIP₂ into IP₃ and DAG. This critical process initiates calcium influx and the polarized mobilization of cytotoxic granules towards the target cell. PLCG2 is expressed in hematopoietic cells and is related to the ubiquitously-expressed PLCG1. While PLCG1 is sufficient for many signaling pathways including mitogen and TCR signaling, PLCG2 is uniquely necessary for NK cell and B cell signaling, as demonstrated by their profound disruption in PIcg2−/− mice.

Mutations in PLCG2 have been previously reported in a separate domain (the C-terminal SH2 domain, cSH2), causing the syndromes PLAID and APLAID. PLAID is caused by exonic deletions compromising the cSH2 domain and results in gain-of-function at sub-physiologic temperatures. At normal temperatures, PLAID acts in a dominant-negative manner to dysregulate immune signaling. While NK cell degranulation is reduced in PLAID, B cell and mast cell dysregulation underlie the predominant clinical phenotypes in these patients, including antibody deficiency and cold urticaria. APLAID is caused by a constitutive gain-of-function mutation (S707Y) and results in autoinflammation and B cell immunodeficiency (perhaps as a result of substrate depletion). Herein is demonstrated that heterozygous loss-of-function mutations in PLCG2 result in a clinical phenotype distinct from PLAID and APLAID, extending the spectrum of disease seen with human mutations in PLCG2.

Methods

Patients and Sample Collection

All human samples were obtained using written informed donor consent and were used with the approval of either the Washington University School of Medicine Institutional Review Board or the Baylor College of Medicine Institutional Review Board for the Protection of Human Subjects. All samples were obtained in compliance with the Declaration of Helsinki. Peripheral blood mononuclear cells (PBMC) were isolated by density centrifugation over Ficoll-Paque according to manufacturers instructions prior to liquid nitrogen cryopreservation in fetal bovine serum and DMSO.

Animal Studies

Plcg2 mice were generated as previously described and backcrossed onto a B/6 background for 10 generations. Mice were maintained under specific pathogen-free conditions and used between 8 and 14 weeks of age. Mouse experiments were performed in both male and female mice with equivalent results. All experiments were approved and conducted in accordance with Washington University Animal Care and Use Committee guidelines for animal care and use.

Exome Sequencing

For kindred A, exome sequencing was performed as previously described. Briefly, genomic DNA was extracted from whole blood or saliva and coding regions enriched using SureSelect All Exon (Agilent Technologies) followed by next-generation sequencing (Illumina) at St. Louis Children's Hospital through the Genome Technology Access Center at Washington University. For kindred B, whole exome sequencing was performed on genomic DNA by Baylor Genetics as previously described. After quality control, alignment and variant calling, exome data was analyzed using institution-specific pipelines to identify potential variants. Medium or high impact variants (i.e., non-synonymous changes, early stops, frameshifts and splice site mutations) with minor allele frequencies less than 0.01 in ExAC6 were prioritized and potential variants were cross-referenced between kindreds. Both kindreds were negative for variants in known genes associated with immunodeficiency and immunodysregulation, including HLH mutations and NK cell associated mutations in MCM4, GATA2, IRFB, FCGR3A (CD16) and KLRC2 (NKG2C).

Transfection Experiments 293T cells (ATCC CRL-3216) were cultured in Dulbecco's modified eagle media supplemented with 10% fetal bovine serum. Cells were transfected with a mammalian expression vector driven under MND promoter containing N-terminal FLAG tagged PLCG2 (WT or relevant mutation) using Lipofectamine LTX (Invitrogen) and incubated for 24 hours. Cells were then stimulated with 100 μM pervanadate and either lysed with RIPA buffer or fixed using 1.6% formaldehyde 15 minutes post-stimulation. Western blot analysis of protein expression was performed using anti-FLAG (Sigma, clone M2) or Actin (Santa Cruz Biotechnology, clone C-11). PLCG2 phosphorylation was analyzed by flow cytometry as described below using PE anti-PLCG2 Y759 (clone K86-689.37, BD).

NK Cell Cytotoxicity Assays

PBMCs isolated as described above were thawed and allowed to rest for 1 hour in Roswell Park Memorial Institute (RPMI) media supplemented with 20% FBS, pyruvate, non-essential amino acids, glutamine and HEPES. Cells were then seeded into 96 well U-bottom plates along with CellTrace Violet (Invitrogen) labeled K562 (ATCC, CCL-243) tumor target cells at a PBMC:K562 ratio of 50:1. After incubation for 4 hours, 7-AAD (BD) was added and target cell death was quantified using flow cytometry. Mouse NK cell assays were performed similarly except that NK cells were isolated from spleen using EasySep Mouse NK Cell Enrichment (Stem Cell Technologies) and then mixed with CellTrace Violet labeled YAC-1 (ATCC, TIB-160) or RMA-S cells.

Microscopy

For analysis of NK-target cell conjugates, fixed cell confocal microscopy was performed using patient NK cells and K562 erythroblast target cells. 3×10⁶ PBMC from patient or heathy donor were incubated with K562 target cells for 45 minutes then fixed, permeabilized and stained with anti-perforin Alexa Fluor 488 (clone dg9), anti-tubulin biotin followed by Pacific Blue conjugated streptavidin, and phalloidin Alexa Fluor 568. Images were acquired on a Zeiss AxioPlanll with a Yokogawa CSU-10 spinning disk and Hamamatsu ORCA-ER camera. Excitation lasers (405 nm, 488 nm, 561 nm, 647 nm) were merged through a

Spectral Applied Research laser merge. Images were taken throughout the volume of cell conjugates using 0.5 μm steps. Acquisition and analysis were performed using Volocity software (PerkinElmer). For measurement of actin accumulation, analysis was performed as described previously. Briefly, area and intensity of F-actin at the immunological synapse were measured for a defined area. Cortical F-actin intensity from both NK and target cells was subtracted from this measurement to generate a quantitative measure of specifically accumulated actin at the synapse. For MTOC polarization, MTOC were defined as the highest intensity staining of a-tubulin and the distance between this and the center of the immunological synapse was measured for 30 conjugates each from patient and healthy donor. Granule convergence was analyzed as previously described. Distance between individual lytic granules and the MTOC were measured and the mean of these was calculated for each cell.

Mass Cytometry

Mass cytometry is a high-dimensional single cell analysis technique based on flow cytometry but differs in its use of metal tagged antibodies in lieu of fluorophores. Procedure performed as described previously and acquisition was performed using a CyTOF instrument (Fluidigm). Data was analyzed and viSNE was performed using Cytobank. Briefly, for TABLE 1, PBMCs were isolated, thawed and rested as detailed above.

TABLE 1
CyTOF panel for PBMC subpopulation and signaling analysis

| Antigen | Clone | Tag |
|---|---|---|
| CD45 | HI30 | 089Y |
| CCR6 | G034E3 | 141Pr |
| CD19 | HIB19 | 142Nd |
| CD45RA | HI100 | 143Nd |
| pPLCG2 | K86-689.37 | 144Nd |
| CD4 | RPA-T4 | 145Nd |
| IgD | IA6-2 | 146Nd |
| CD11c | Bu15 | 147Sm |
| CD16 | 3G8 | 148Nd |
| CD127 | A019D5 | 149Sm |
| pSTAT5 | 47 | 150Nd |
| CD123 | 6H6 | 151Eu |
| pAKT | D9E | 152Sm |
| pSTAT1 | 58D6 | 153Eu |
| pBtk/Itk | 24a/BTK | 154Sm |
| CD27 | L128 | 155Gd |
| CXCR3 | G025H7 | 156Gd |
| pSTAT3 | 4/P-Stat3 | 158Gd |
| pMAPKAPK2 | 27B7 | 159Tb |
| CD14 | M5E2 | 160Gd |
| CD80 | 2D10.4 | 161Dy |
| pLCK | 4/LCK-Y505 | 162Dy |
| pJAK2 | D4A8 | 163Dy |
| IkBa | L35A5 | 164Dy |
| CD45RO | UCHL1 | 165Ho |
| pNFKB | K10- | 166Er |
| | 895.12.50 | |
| pERK | D13.14.4E | 167Er |
| CD8 | SK1 | 168Er |
| CD25 | 2A3 | 169Tm |
| CD3 | UCHT1 | 170Er |
| pZAP70 | 17a | 171Yb |
| IgM | MHM-88 | 172Yb |
| HLA-DR | L243 | 173Yb |
| pSTAT4 | 38/p-Stat4 | 174Yb |
| PD-1 | EH12.2H7 | 175Lu |
| CD56 | NCAM16.2 | 176Yb |
| CD11b | ICRF44 | 209Bi |

Cells were stained with cisplatin to track cell viability. Between 1×10⁶ and 3×10⁶ PBMCs per time point were stained with metal conjugated extracellular antibodies (Fluidigm), seeded in 96-well U bottom plates and stimulated with either a cocktail of A) 500U/mL IFNα Peprotech), 500 ng/mL LPS (Invivogen), 50ng/mL IL-12 (Peprotech), and 500U/mL IL-2 (Proleukin), and CD16/CD3/IgM crosslinking (using surface staining antibodies followed by anti-mouse IgG, Biolegend) for 0, 3 and 15 minutes. Cells were then fixed in 1.6% formaldehyde and permeabilized in 100% methanol. After washes, intracellular staining was performed before DNA staining using Cell-ID Intercalator-Ir (Fluidigm). For the panel in TABLE 2, cells were stained with cisplatin to track cell viability.

TABLE 2
CyTOF panel for NK cell development and function.

| Antigen | Clone | Tag |
|---|---|---|
| CD45 | HI30 | 89 Y |
| Barcoding | Barcoding | Barcoding |
| KIR2DL4 (CD158d) | 181703 | 141 PR |
| CD19 | HIB19 | 142 Nd |
| KIR3DS1/L1 (CD158e1, e2) | Z27 | 143 Nd |
| CD3 | UCHT1 | 144 Nd |
| KIR2DS4 (CD158i) | FES172 | 145 Nd |
| KIR2DL1/DS1 | EB6B | 146 Nd |
| (CD158a, h) | | |
| NKG2D | 1D11 | 147 Sm |
| KIR2DL2/2DL3 (GD158b) | CH-L | 148 Nd |
| CD127 | A019D5 | 149 Sm |
| MIP-1a | 93342 | 150 Nd |
| CD107a | H4A3 | 151 Eu |
| TNF-a | Mab11 | 152 Sm |
| CD62L | DREG-56 | 153 Eu |
| KIR2DL5 (CD158f) | UP-R1 | 154 Sm |
| CD27 | L128 | 155 Gd |
| KIR3DL1 (CD158e) | DX9 | 156 Gd |
| CD137 | 4B4-1 | 158 Gd |
| NKG2C | 134591 | 159 Tb |
| CD69 | FN50 | 160 Gd |
| NKp30 | P30-15 | 161 Dy |
| Ki67 | B56 | 162 Dy |
| CD94 | DX22 | 163 Er |
| CD16 | 3G8 | 165 Ho |
| NKG2A | Z199 | 166 Er |
| NKp44 | P44-8 | 167 Dy |
| IFN-g | B27 | 168 Er |
| CD25 | 2A3 | 169 Tm |
| NKp80 | 239127 | 170 Er |
| GzmB | GB11 | 171 Yb |
| CD57 | HCD57 | 172 Yb |
| CXCR6 | K041E54 | 173 Yb |
| NKp46 | 195314 | 174 Yb |
| Perforin | B-D48 | 175 Lu |
| CD56 | HCD56 | 176 Yb |
| CD11b | ICRF44 | 209 Bi |
| DNA | intercalator-IR | 191/193 |
| Cisplatin | NA | 194/195 Pt |

Between 1×10⁶ and 3×10⁶ PBMCs were unstimulated or mixed with 1:1 K562 cells and 500 U/mL IL-2 (Proleukin) in the presence of GolgiStop (BD), GolgiPlug (BD) and metal-conjugated CD107 antibody (Fluidigm). Cells were then stained with conjugated extracellular antibodies (Fluidigm) or antibodies conjugated to the desired metal using Maxpar Antibody Labeling Kit (Fluidigm). Cells were fixed in CytoFix/CytoPerm (BD), washed in Perm Buffer (eBioscience) and stained with intracellular antibodies before DNA staining using Cell-ID Intercalator-Ir (Fluidigm).

Flow Cytometry

Where indicated, flow cytometry was performed using either surface staining alone at room temperature for 15 minutes or surface staining in combination with methanol permeabilized intracellular staining at room temperature for 60 minutes before acquisition using a Fortessa X-20 (BD). Data analysis performed using Cytobank or FlowJo. Human antibodies used in this study: APC CD56 (clone 5.1H11, Biolegend), Pacific Blue CD3 (clone OKT3, Biolegend), APC Cy7 CD14 (Clone HCD14, Biolegend), FITC NKG2D (clone 1D11, Biolegend), FITC CD16 (clone 3G8, BD), PE NKp44 (clone P-44-8, Biolegend), PE 2B4 (clone C1.7, Biolegend), PE-Cy7 CD19 (clone H1B19, Biolegend), APC IgM (clone MHM-88, Biolegend), APC-Cy7 HLA-DR (clone L243, Biolegend), and Alexa Fluor 488 total PLCG2 (clone K86-1161, BD). Mouse antibodies used in this study: BV786 CD45 (clone 30-F11, BD), BV421 NK1.1 (clone PK136, BD), BV510 IgM (clone R6-60.2, BD), FITC CD11 b (clone M1/70, Biolegend), PE-Cy7 CD27 (clone LG3A10, Biolegend), PE CD4 (clone GK1.5, Biolegend), PerCP IgD (clone 11-26c.2a, Biolegend), APC B220 (clone RA3-6B2, BD), APC-Cy7 CD3 (clone 17A2, Biolegend), APC-R700 CD8 (clone RPA-T8, BD), FITC CD43 (clone S11, Biolegend), PE CD24 (clone 30-F1, Biolegend), PE-Cy7 CD11b (clone M1/70, BD), PE Ly49H (clone 3D10, BD), and APC-Cy7 CD19 (clone 1D3, BD).

ELISA

Human serum IgG and IgM levels were analyzed using commercially available ELISA kits (Invitrogen) according to manufacturer instructions. Briefly, ELISA plates were coated with capture antibody overnight before washing, blocking and incubation with diluted patient sera or standard. Plates were then washed again, incubated with HRP-conjugated detection antibody, washed again and incubated with TMB substrate solution. Solution was then stopped with 1 M phosphoric acid and absorbance was measured at 450 nm.

Calcium Flux Analysis

For human samples, NK cells were enriched using RosetteSep Human

NK Cell Enrichment (Stem Cell Technologies). 1-2x10⁶ enriched NK cells were then loaded with Indo-1 dye (Invitrogen) and labelled with PE or FITC conjugated mouse IgG antibodies against the NK cell receptors 2B4 and NKG2D. Kinetic measurements of calcium flux were obtained using a BD Fortessa X-20 at baseline and then upon antibody crosslinking using anti-mouse IgG. Mouse calcium flux analysis was similarly performed: NK cells were isolated from spleen using EasySep Mouse NK Cell Enrichment (Stem Cell Technologies), loaded with Indo-1, labelled with APC NK1.1 followed by crosslinking and acquisition as above. Patient B.ll.4 was performed similarly except that expansion beforehand was required due to limited patient sample and anti-NKp44 and anti-NKG2D were used for crosslinking. NK cells were expanded as previously described. Briefly, 10⁶ PBMCs from patient B.ll.4 or healthy control were co-incubated with 10⁶ irradiated (100 Gy) K562-mbIL15-41bbl for 7 days. After 7 days, cells were removed and assessed for purity. T cells (CD3+) were present at less than <1%. 100 U/mL of recombinant IL-2 (Proleukin) was added to the culture and incubated for 7 more days with partial media exchange every 2 days. After 14 days total, NK cells expanded 10 to 15-fold with >95% purity (CD56+CD3-) and were then used for cytotoxicity and calcium flux assays. For murine B cell calcium flux analysis, naive B cells were gated on in whole splenocytes (B220+CD27−) and treated as above, with the exception of using anti-mouse IgM as the crosslinking antibody. Human B cell calcium flux analysis was performed from PBMCs (gated as CD19+) with the exception of using anti-human IgM as the crosslinking antibody.

Statistics

Normal internal reference ranges for human NK cell cytotoxicity and mass cytometry were determined using 25 healthy controls; outliers were removed using ROUT (Robust regression and Outlier removal) and the central 95th percentile was determined. Upper and lower bounds are visualized by dashed lines. Unless normality was established after D'Agostino & Pearson omnibus test for normality, pairwise comparisons are made using Mann-Whitney U test with

Bonferroni correction for multiple comparisons. Where noted, comparisons between healthy controls and patients are performed using age and gender matched healthy control donors. All statistics performed using Graphpad Prism.

Molecular Dynamics, Structural Analysis, and Conservation Analysis

Structural diagrams were generated using PyMOL v2.0 (PyMOL Molecular Graphics System). Conservation analysis of the G595 and L183 residues were generated using M-Coffee with ESPript secondary structure analysis. For molecular dynamics, 134.9 μs of aggregate simulation time of the SH2 domain (107 aa) with wild-type (69.4 μs) and G595R mutant (65.5 μs) sequences was ran with GROMACS 2016.1 at 300K using the AMBER03 force field with explicit TIP3P solvent. Salt was added to neutralize the system and create a solution concentration of 100 mM (13 Na⁺/14 CI⁻ for wild-type, 16 Na⁺/18 CI⁻ for G595R). Simulations were prepared by placing the starting structure for each sequence in a dodecahedron box that extended 1.0 A beyond the protein in any dimension. Each system was then energy minimized with the steepest descent algorithm until the maximum force fell below 100 kJ/mol/nm using a step size of 0.01 nm and a cutoff distance of 1.2 nm for the neighbor list, Coulomb interactions, and van der Waals interactions. For production runs, all bonds were constrained with the LINCS algorithm and virtual sites were used to allow a 4fs time step. Cutoffs of 1.0 nm were used for the neighbor list, Coulomb interactions, and van der Waals interactions. Before being run in production, systems were equilibrated with position restraints for all heavy atoms for 1 ns. The Verlet cutoff scheme was used for the neighbor list. The stochastic velocity rescaling (v-rescale) thermostat was used to hold the temperature at 300 K. Conformations were stored every 10 ps. The initial structure for both simulations was a homology model of Swiss Model threading the human PLCG2 sequence onto 4EYO, a crystal structure of the close human paralog, PLCG1. The human paralog was used (rather than the murine homolog) because the crystal structure contains both nSH2 and cSH2 domains and so provided more information about the course of the C-terminal amino acids of the nSH2 domain than the murine homolog structure 2DX0. A microstate decomposition was built using khybrid clustering with a radius of 1.5 Å and 5 rounds of kmedoids updates on the entire 135 μs dataset using backbone atoms (C, Cα, N, O) and Cβ (except residue 595) to produce 2314 states. Using the cluster centers derived in this way, the data for the wild-type and the mutant was reassigned separately and generated separate Markov state models on this shared state space. Transition probabilities were fit with the transpose method. The state space had near complete coverage for both sequences with the wild-type sampling 2204/2315 states and the mutant sampling 2150/2315 states. The lagtime was 1.5 ns and was determined using the implied timescales test.

Results

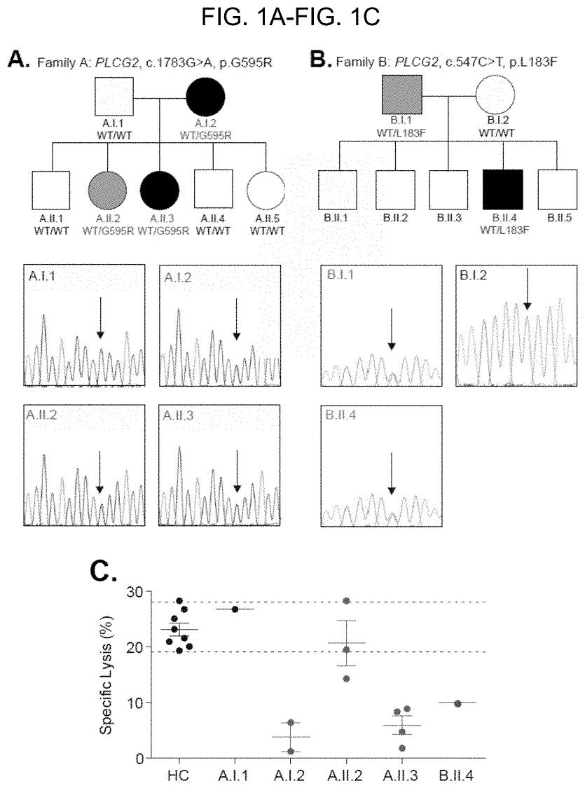

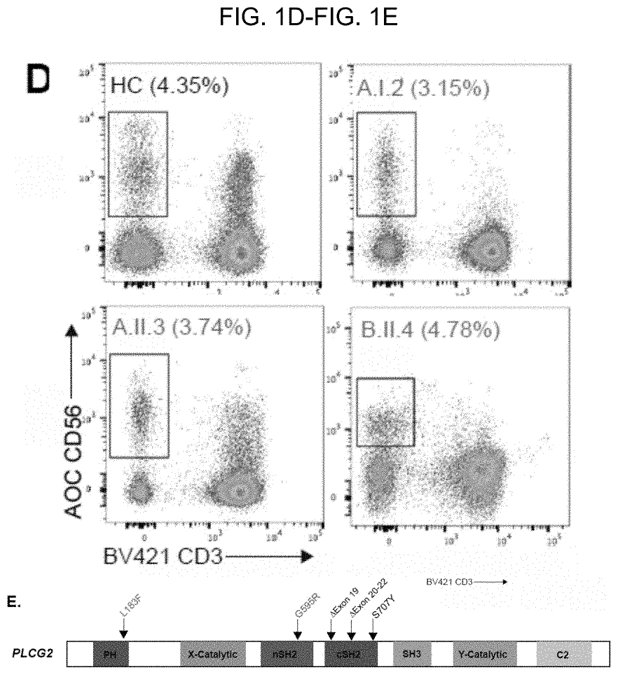

Patients were identified from two unrelated nonconsanguineous families with autosomal dominant immunodeficiency, characterized by recurrent infections and reduced NK cell killing. In family A (see e.g., FIG. 1A), patient A.I.2 is a CMV/HSV1-seronegative 52-year-old Caucasian female with a history of arthralgias, antiphospholipid syndrome, and late-onset recurrent Staphylococcal septicemia. Her daughter, A.II.3, is a 19-year-old female with a history of arthralgias and autoimmunity (positive antinuclear antibody and type 1 diabetes), as well as recurrent HSV1 gingivostomatitis requiring prophylactic valacyclovir. Family B (see e.g., FIG. 1B) consists of patient B.II.4, a 9-year-old

Qatari male with a history of CMV myocarditis, as well as adenoviral hepatitis. There was no history of immunodeficiency or autoimmunity in any other family members. Clinical NK cell testing in both families revealed reduced NK cell K562 killing, despite intact CD107 degranulation against K562 cells and normal cytotoxic granule contents (see e.g., FIG. 1C and TABLE 3).

TABLE 3
Clinical characteristics and phenotypes
of PLCG2 haploinsufficiency patients.

| | Patient | Patient | Patient |
|---|---|---|---|
| | A.I.2 | A.II.3 | B.II.4 |
| Mutation | G595R | G595R | L183F |
| Absolute | Normal | Normal | Normal |
| Lymphocyte | | | |
| Count* | | | |
| Absolute | Normal | Normal | Normal |
| Neutrophil | | | |
| Count* | | | |
| Herpesvirus | None | HSV1 | CMV |
| Infections | | Gingivostomatitis | Myocarditis |
| Bacterial | Recurrent Sepsis | None | None |
| Infections | | | |
| Hepatitis, | Negative | Negative | Positive |
| Unknown Origin | | | |
| HSV1 Serology* | Negative | Positive | Negative |
| CMV Serology* | Negative | Positive | Positive |
| Antinuclear | Positive | Positive | Negative |
| Antibody* | | | |
| Other | Antiphospholipid | Type I Diabetes | None |
| Autoimmunity | Syn. | | |
| Absolute NK Cell | Normal | Normal | Normal |
| Count* | | | |
| NK Cell | Reduced | Reduced | Reduced |
| Cytotoxicity* | | | |
| NK Cell | Normal | Normal | Normal |
| Perforin/Granzyme* | | | |
| NK Cell CD107 | Normal | Normal | Normal |
| Degranulation* | | | |
| NK Cell Maturity | Increased | increased | Increased |
| (CD57⁺) | | | |
| NKG2C⁺ NK Cells | (CMV | Negative | Negative |
| | Seronegative) | | |
| Monocytes/DCs | Reduced | Reduced | Reduced |
| T_{FH} Phenotype | T_{FH2} > T_{FH1} | T_{FH2} > T_{FH1} | T_{FH2} > T_{FH1} |
| B Cell Count* | Reduced | Reduced | Normal |
| Class Switched | Normal | Normal | Normal |
| Memory B Cells | | | |
| IgA/IgG/IgM* | Normal | Normal | Normal |
| IgE* | Not Evaluated | Normal | Normal |
| Pneumococcal | Normal | Not Evaluated | Normal |
| Antibody | | | |
| Titers* | | | |
| T Cell Mitogen | Not Evaluated | Not Evaluated | Normal |
| Stimulation* | | | |

*Measured in clinical laboratory

Flow cytometry of peripheral blood demonstrated normal NK cell percentages and absolute counts inconsistent with classical NKD (see e.g., FIG. 1D and TABLE 3). Further clinical immunology evaluation of immunoglobulin levels (IgM, IgG, IgA, and IgE), protective antibody titers, T cell mitogen stimulation, and immune subpopulation analysis was also unremarkable (see e.g., TABLE 3). Patients and unaffected relatives underwent whole-exome sequencing which revealed novel heterozygous PLCG2 missense variants.

Patients A.I.2 and A.II.3 possessed heterozygous mutations (c.1783G>A, p.G595R) in PLCG2, located in the N-terminal SH2 domain (nSH2). An additional healthy HSV1-seropositive 17-year-old sibling (A.II.2) with the mutation was identified; however, her borderline-normal NK cell killing suggests incomplete penetrance, a common feature of autosomal dominant immune syndromes. Patient B.ll.4 possessed a different heterozygous mutation in PLCG2 (c.547C>T, p.L183F), located in the Pleckstrin homology (PH) domain. Patient B.l.1 also possessed this mutation but was not available for evaluation. The locations of these mutations and other reported PLCG2 mutations are diagrammed in FIG. 1E.

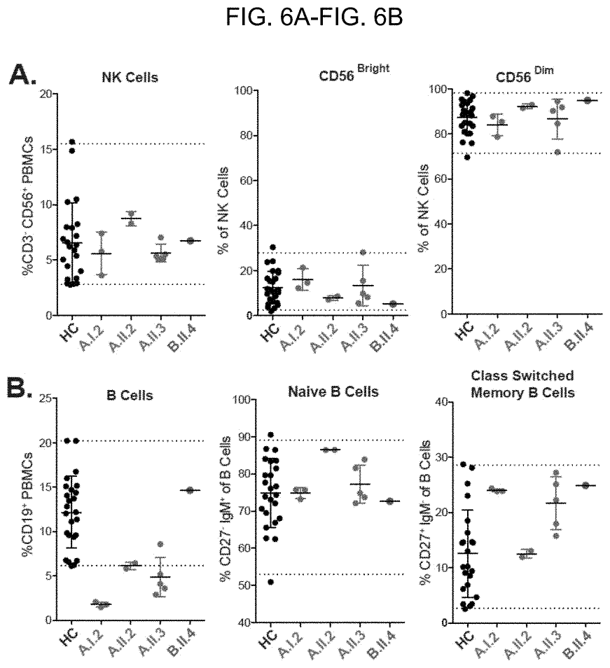

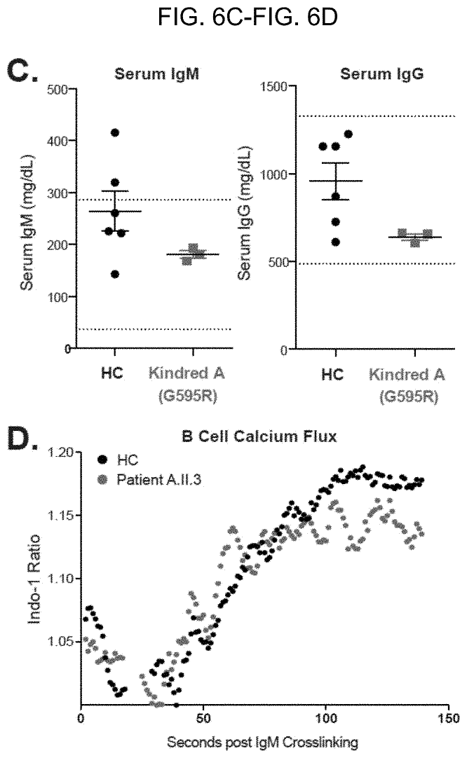

As immunodeficiencies commonly arise from aberrant immune cell development or signaling, mass cytometry (CyTOF, see e.g., TABLE 1) was employed to analyze these processes in the peripheral blood. Consistent with clinical studies, NK cell abundance, as well as the distributions of immunomodulatory CD56^{Bright} and cytotoxic CD56^{Dim} NK cells, were intact (see e.g., FIG. 6A).

Family A demonstrated reduced B cells with preserved naïve to class-switched memory B cell percentages, suggesting a defect in B cell output but not activation (see e.g., FIG. 6B). In support of this, serum immunoglobulins, seroconversion, and IgM-induced calcium flux were normal (see e.g., FIG. 6C,

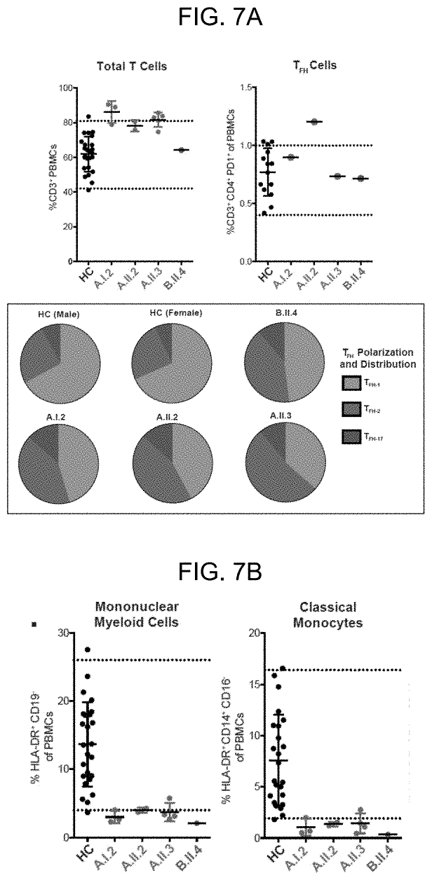

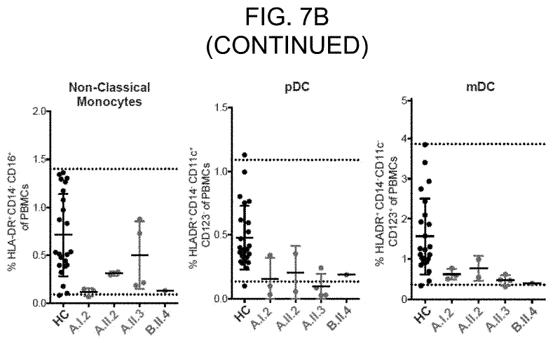

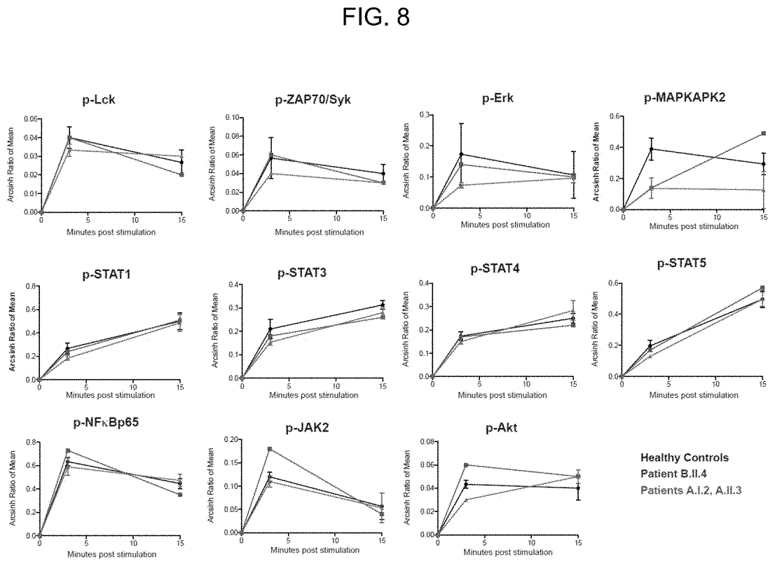

FIG. 6D, and TABLE 3). Although T cell development and calcium flux were unperturbed (data not shown), the distribution of T follicular-helper cells (T_{FH}) was altered with increased T_{FH2} cells and decreased T_{FH}, cells in both families (see e.g., FIG. 7A), a pattern seen previously in human autoimmunity. The abundance of monocytes and dendritic cells, but not other myeloid cells (i.e., granulocytes), was reduced in both families as well (see e.g., FIG. 7B and TABLE 3). Human monocyte activation with macrophage colony stimulating factor (MCSF) is dependent on PLCG2 induced calcium flux, possibly contributing to the observed monocytopenia and bacterial susceptibility in patient A.1.217.

CyTOF analysis of NK cell signaling revealed hypophosphorylation of

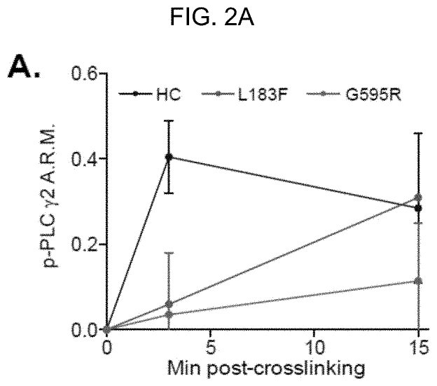

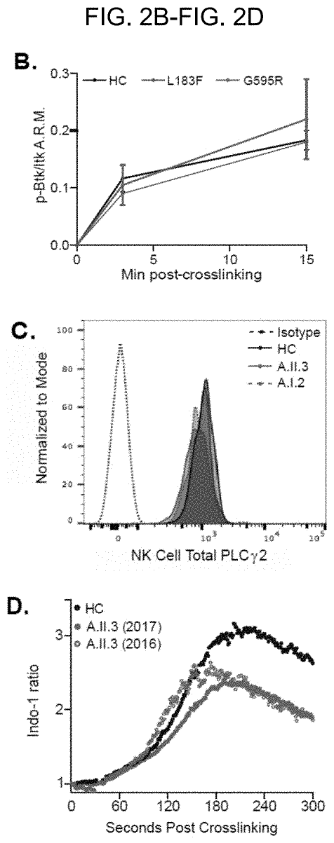

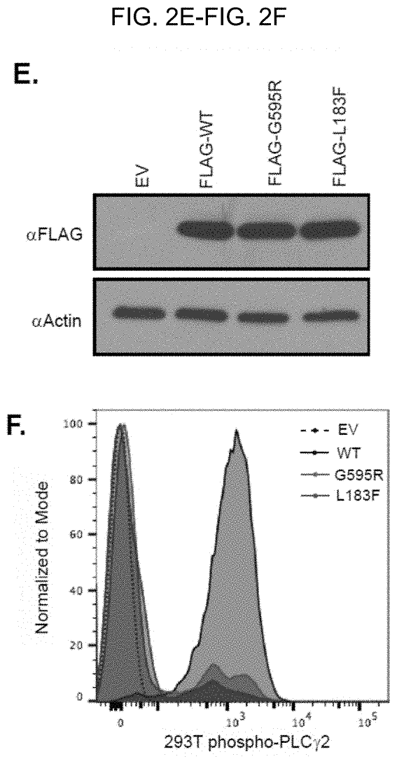

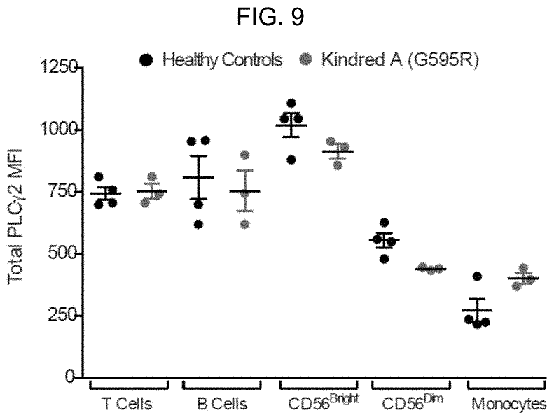

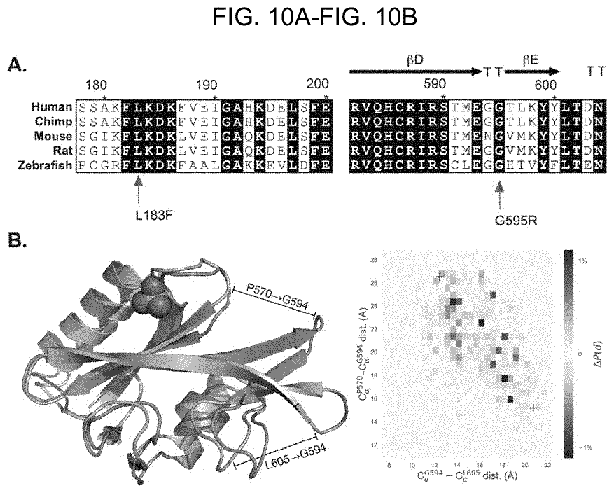

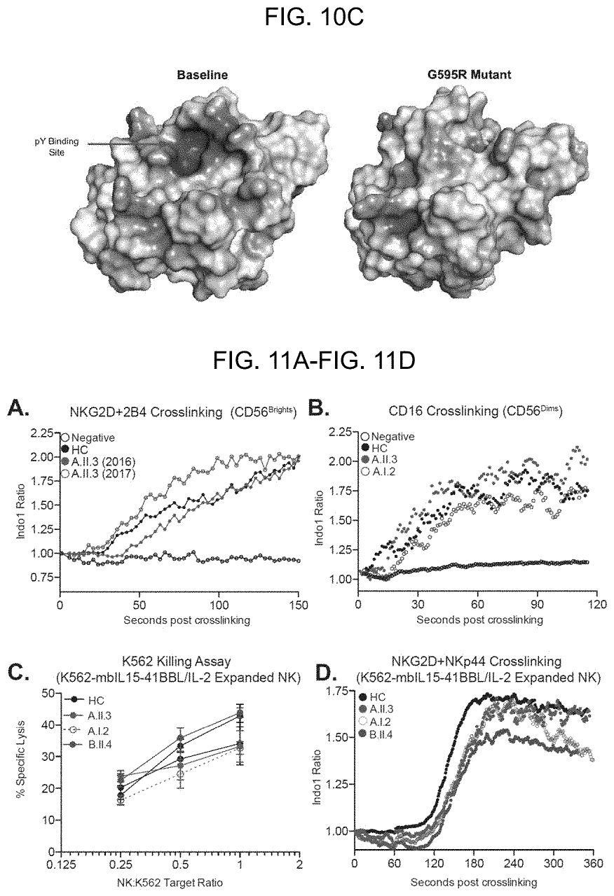

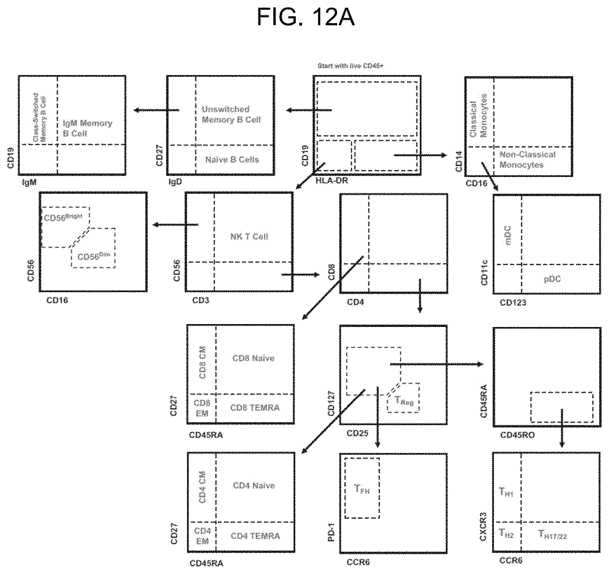

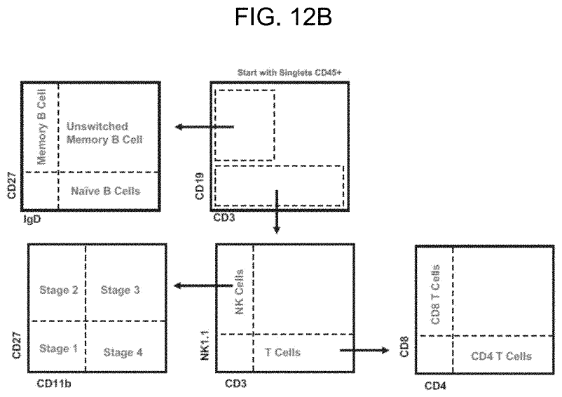

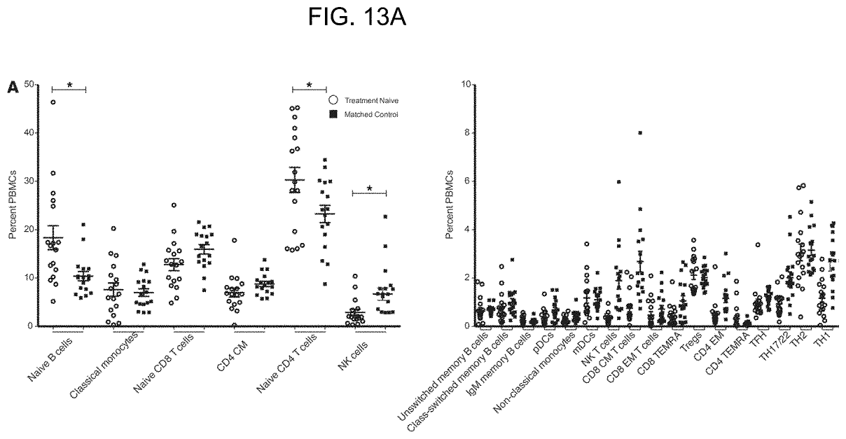

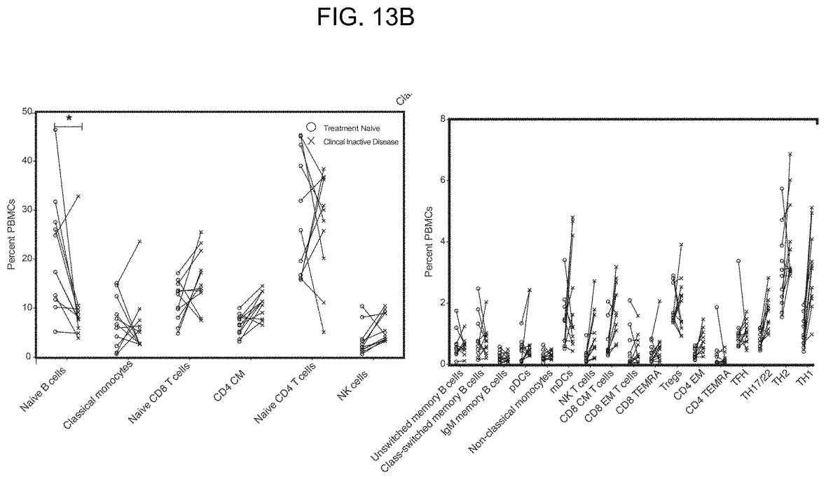

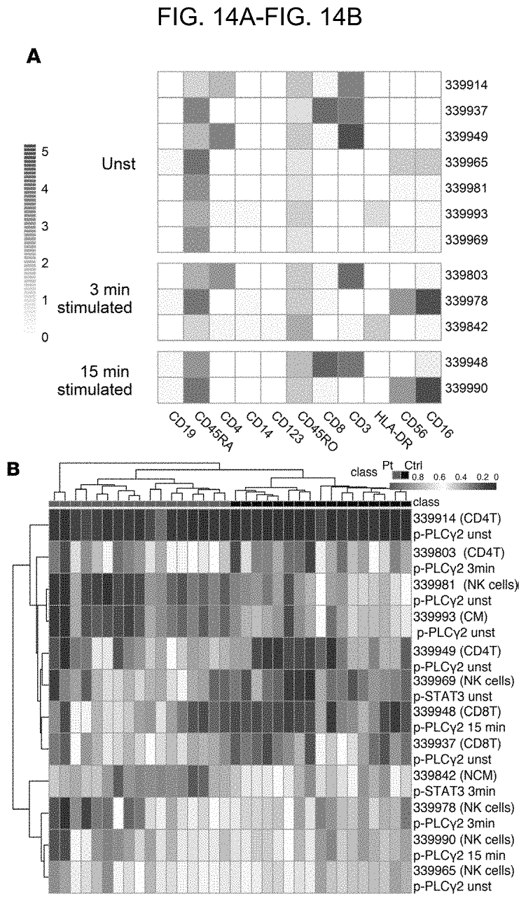

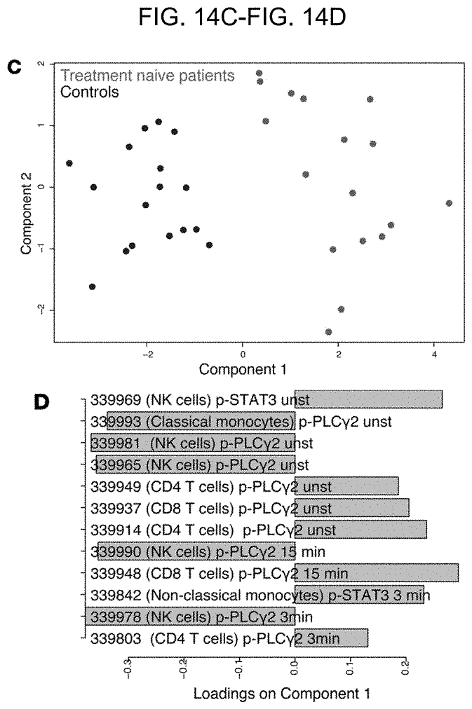

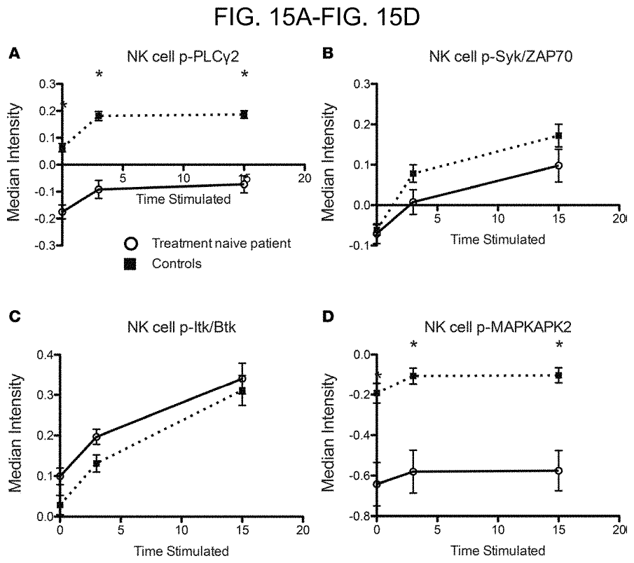

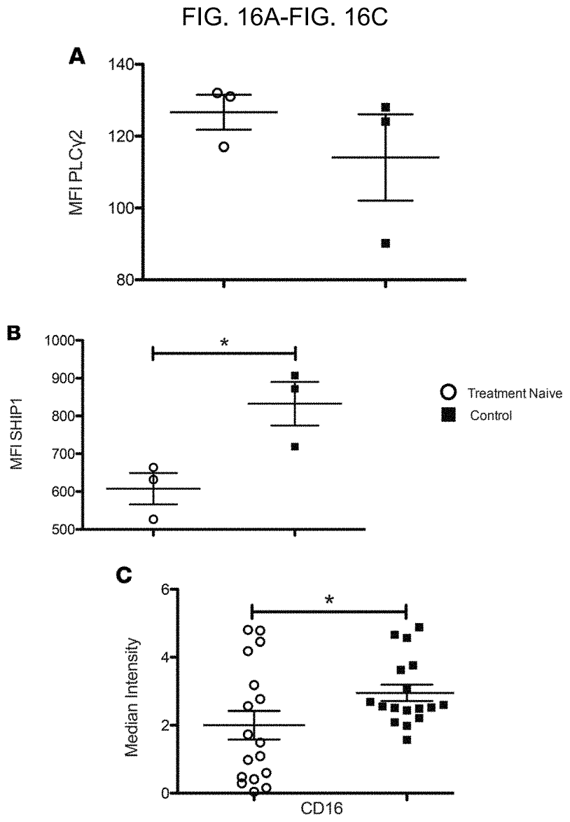

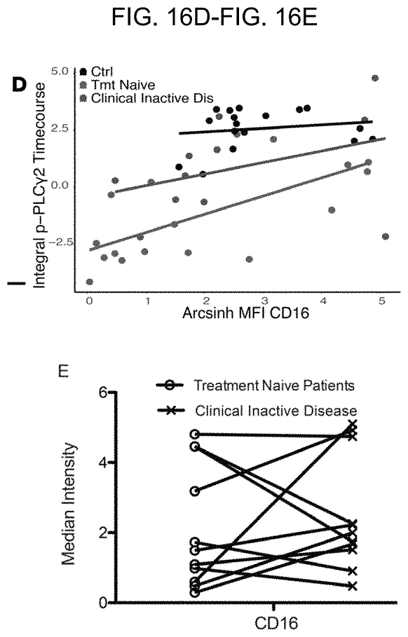

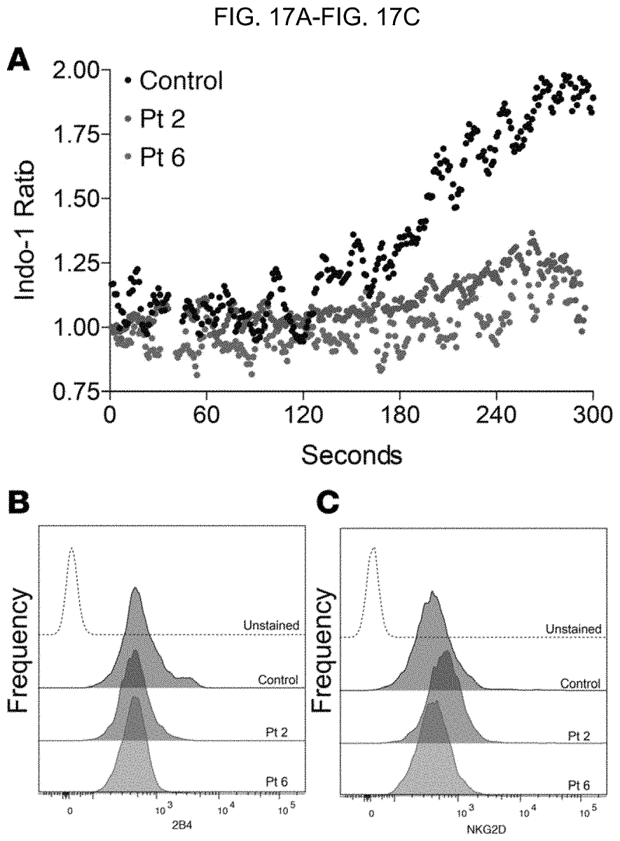

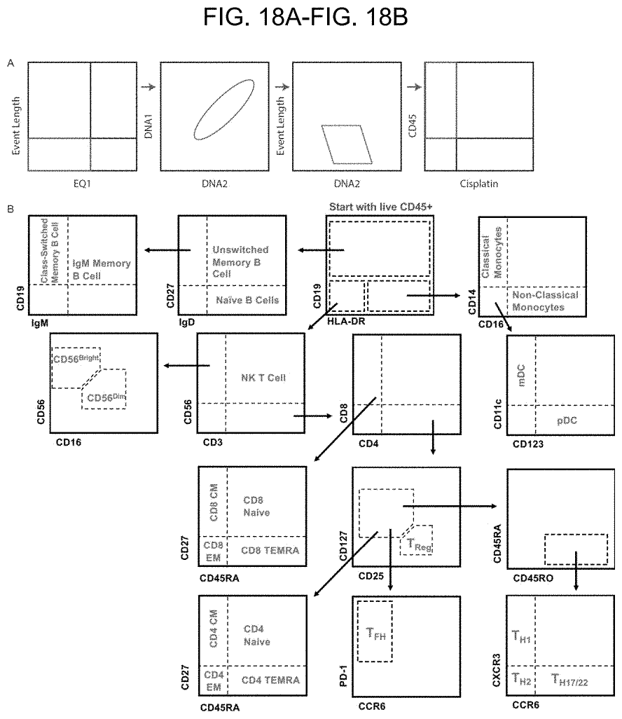

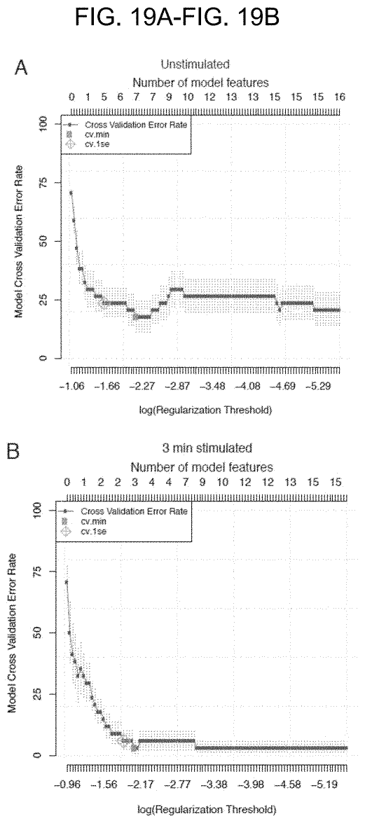

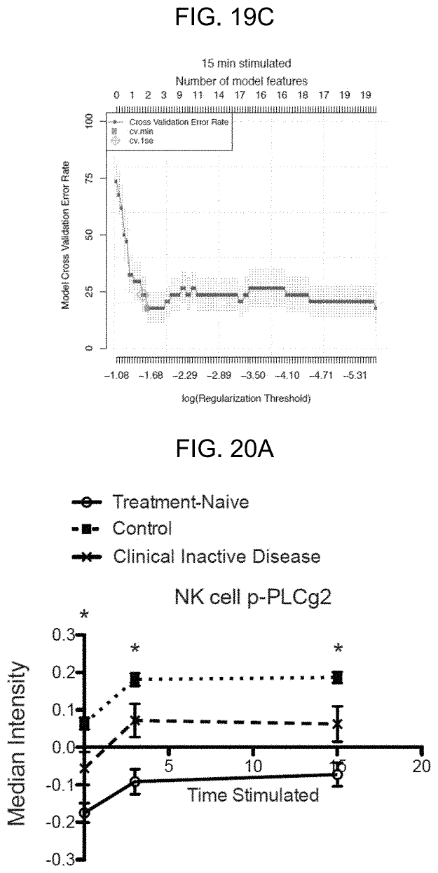

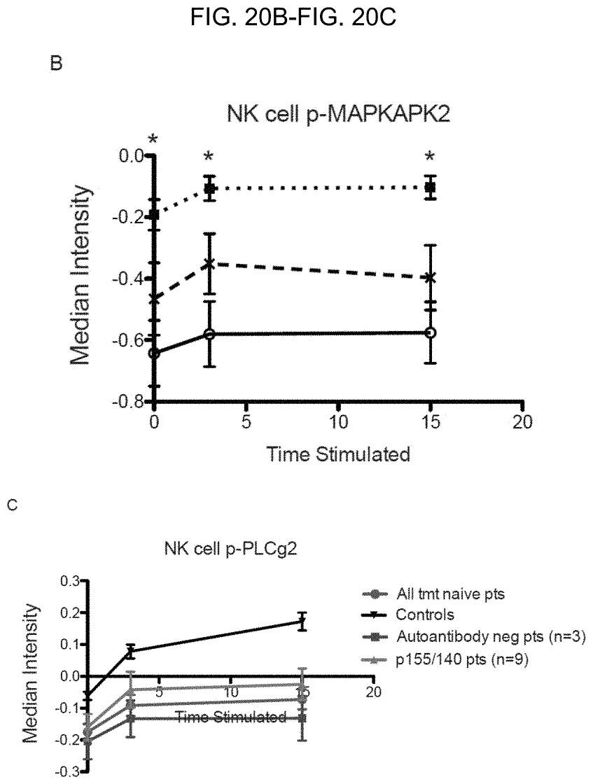

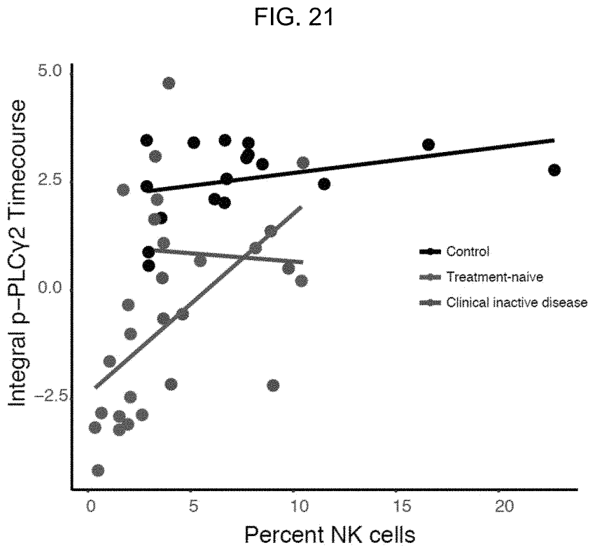

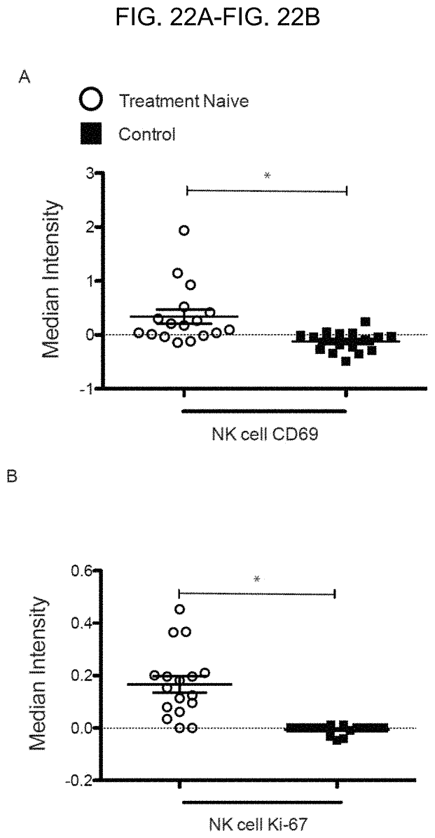

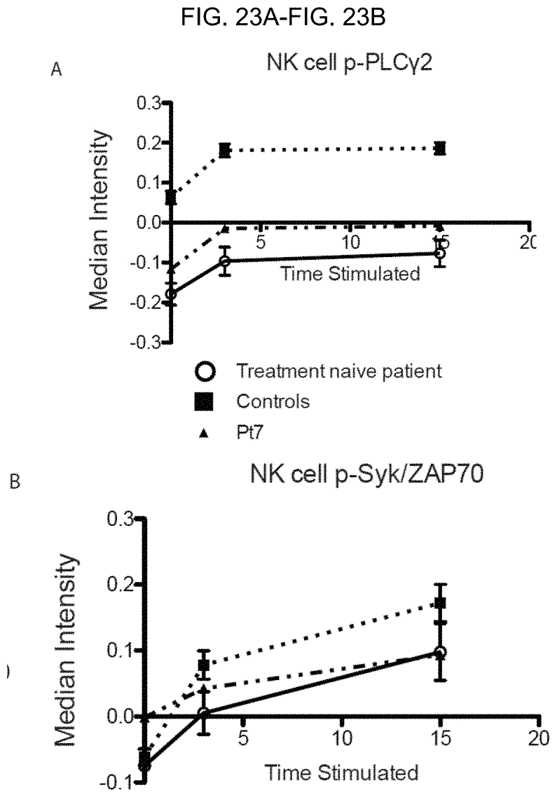

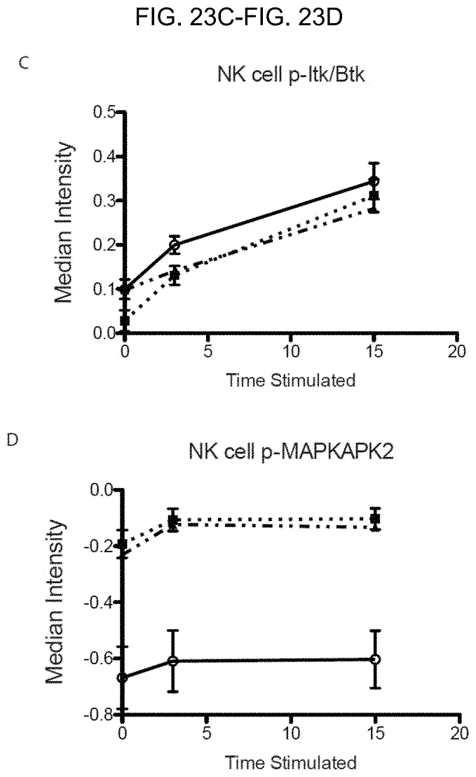

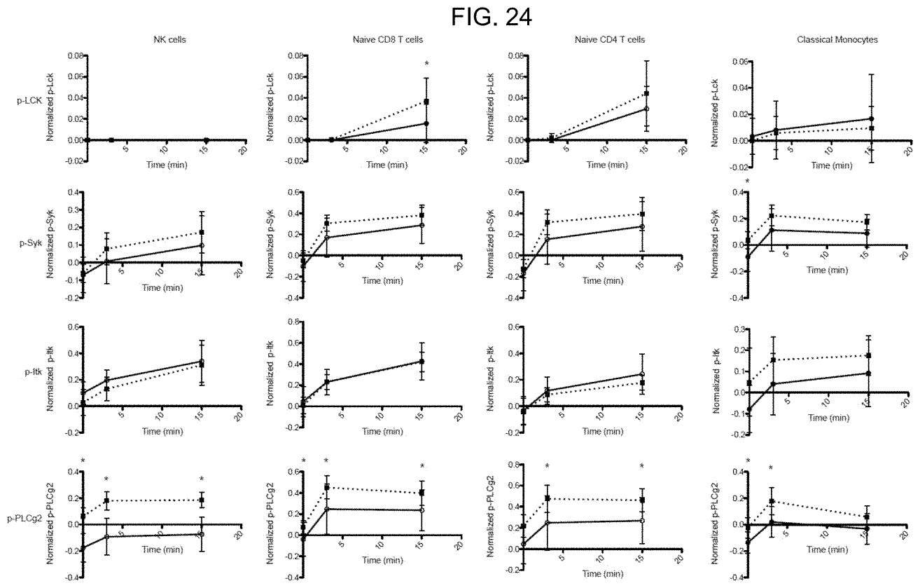

PLCG2 in both families (see e.g., FIG. 2A). Family A demonstrated a reduction in the magnitude of PLCG2 phosphorylation while family B demonstrated altered kinetics. PLCG2 hypophosphorylation was also confirmed by flow cytometry in patients A.I.2 and A.II.3, however, patient A.II.2 had normal PLCG2 phosphorylation (data not shown) consistent with her borderline-normal NK cell killing (see e.g., FIG. 10). Upstream Btk/ltk, ZAP70/Syk, and Lck phosphorylation was intact, suggesting an intrinsic defect in PLCG2 (see e.g., FIG. 2B and FIG. 8). MAPKAPK2, activated by PKC downstream of PLCG2-induced DAG, was similarly hypophosphorylated (see e.g., FIG. 8). Total PLCG2 protein levels in family A were analyzed to establish whether hypophosphorylation was the result of functional inhibition or reduced protein expression. Total PLCG2 protein levels were normal in NK cells (see e.g., FIG. 2C), as well as in all other cell types examined (see e.g., FIG. 9), suggesting that the G595R mutation compromises function and not protein expression. Analysis of PLCG2 protein levels in patient B.II.4 was not feasible due to limited samples. Notably, this analysis also revealed differential PLCG2 expression between immune cell subsets, including physiologically lower PLCG2 expression in monocytes and CD56D¹m NK cells than in T cells, B cells, and CD56^{Bright} NK cells (see e.g., FIG. 9).

Bioinformatic and structural analyses of G595 and L183 suggest that these residues are intolerant to mutation. The G595R and L183F mutations occur at highly-conserved sites in the nSH2 and PH domains of PLCG2, respectively (see e.g., FIG. 10A). Only two other individuals in ExAC are reported to have missense variant at G595, while no missense variants in L183 have been reported. Although no structure exists for the PH domain, nSH2 structures from murine PLCG2 and human PLCG1 facilitated analysis of the G595 mutation with molecular dynamics (MD), which has been used to understand the structural effects of mutations previously. Simulations of wild type and G595R sequences were analyzed by this approach and revealed conformational disturbances in the nSH2 βD-βE loop, potentially compromising the LAT phosphotyrosine binding site (see e.g., FIG. 10B and FIG. 100). In support of the βD-βE loop being critical in SH2 function, a structurally-analogous mutation (G60R) in the loop of SHP-2 has been previously reported as pathogenic and this loop serves as a protein-protein interaction site in the nSH2 of PLCG1.

The catalytic activity of PLCG2 is initiated by phosphorylation, leading to calcium flux and granule movement/polarization. Consistent with PLCG2 hypophosphorylation, calcium flux in patient A.II.3 was stably reduced in CD56^{Dim} NK cells after NKG2D and 2B4 receptor crosslinking (see e.g., FIG. 2D). Notably, CD56^{Bright} NK cells, which express higher levels of PLCG2 protein, demonstrated normal calcium flux with NKG2D+2B4 crosslinking (see e.g., FIG. 9 and FIG. 11A). Calcium flux in CD16-crosslinked NK cells was also normal, consistent with the ability of CD16 to signal through both PLCG1 and PLCG2 (see e.g., FIG. 11 B). Limited patient sample required expansion of patient B.ll.4 NK cells using K562-mbIL15-41 BBL cells and IL-2 before analysis. Patient B.ll.4 NK cells also showed partially reduced calcium flux (see e.g., FIG. 11C and FIG. 11D), although this expansion process largely restored NK cell cytoxicity.

To establish that G595R and L183F are loss-of-function mutations, wild type or mutant FLAG-PLCG2 was expressed in 293T cells (which do not natively express PLCG2) and analyzed for protein expression and pervanadate-induced phosphorylation. Although both mutants were expressed normally (see e.g., FIG. 2E), FLAG-PLCG2^{G595R} and FLAG-PLCG2^{183F} were hypophosphorylated compared to FLAG-PLCG2^{Wildtype} (see e.g., FIG. 2F). Together, these data demonstrate that the G595R and L183F mutations are loss-of-function mutations and likely contribute to functional PLCG2 haploinsufficiency.

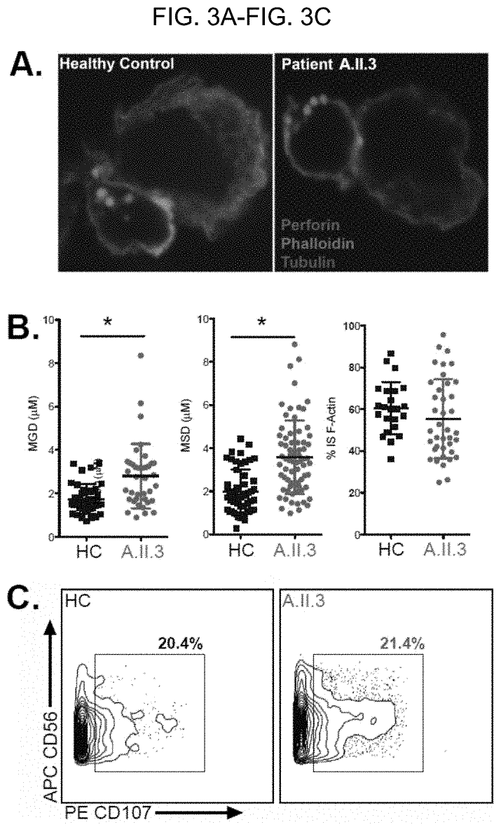

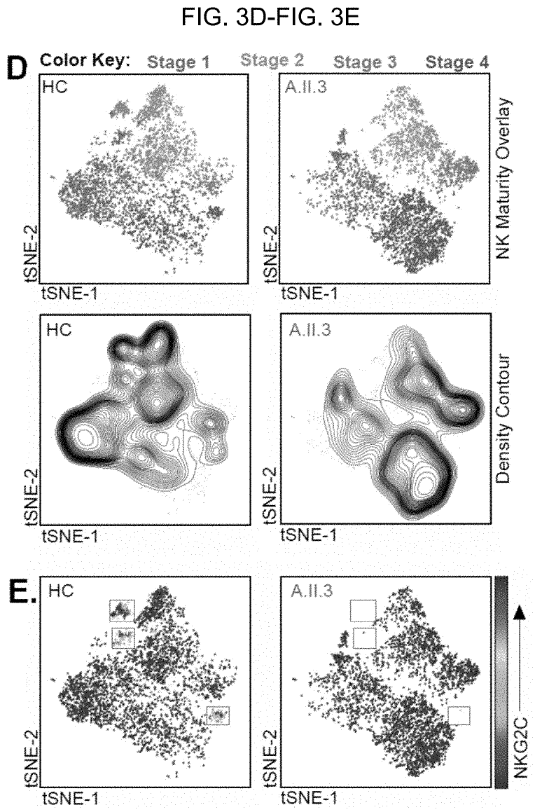

Cytotoxic granule movement was analyzed by microscopy in NK cells conjugated to K562 target cells (see e.g., FIG. 3A). The microtubule organizing center (MTOC) to granule (MGD) and MTOC to synapse (MSD) distances were quantified. Both distances were increased in patient A.II.3, indicating dysregulated cytotoxic granule movement (see e.g., FIG. 3B). Synaptic actin accumulation, regulated independently of PLCG2, was unchanged (see e.g., FIG. 3B). In T cells, calcium flux kinetics and DAG localization influence the path and directionality of granule movement, respectively. The observed defect in NK killing despite intact CD107 degranulation (see e.g., FIG. 3C) suggests that defects in both of these processes may lead to delayed/adirectional degranulation. Although methods to monitor DAG are limited, the defect in MAPKAPK2 phosphorylation downstream of PKC implies that this branch of PLCG2 signaling is also dysregulated (see e.g., FIG. 8).

CyTOF was also used to examine NK cell development and receptor expression (see e.g., TABLE 2). Clustering of NK cells with visual stochastic neighbor embedding (viSNE) enabled visualization of this high-dimensional data, whereby each point represents a cell and groups represent subpopulations which may be identified by marker expression. Activating and inhibitory receptor expression were comparable between patient A.II.3 and control (data not shown); however, patient NK cell density was increased in the viSNE region corresponding to CD57+ maturation stages 3 and 4, indicating increased NK cell maturity (see e.g., FIG. 3D). This phenotype was also noted by flow cytometry in patient B.ll.4 (data not shown). CD57+ acquisition is typically cytokine-driven and associated with increased cytotoxicity, suggesting either persistently elevated cytokine levels (perhaps from increased viral burden) or a potential compensatory mechanism to increase NK cell killing. Additionally, a distinct subpopulation of NKG2C+ NK cells was absent in both patient A.II.3 (see e.g., FIG. 3E) and patient B.ll.4 (data not shown). In most individuals, NKG2C+ NK cells expand during CMV infection and persist thereafter, referred to as the adaptive NK cell response. The absence of this population despite CMV seropositivity in both patients suggests this process may be impacted.

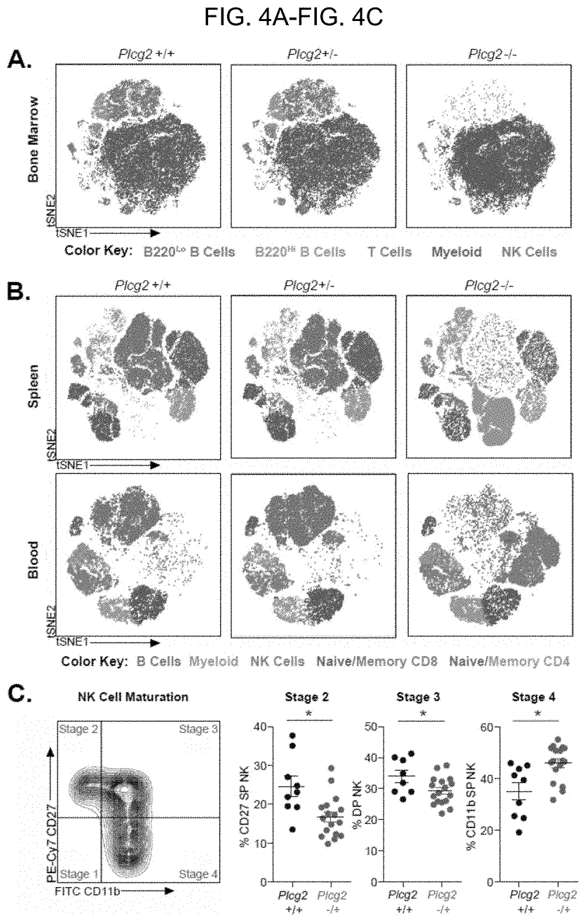

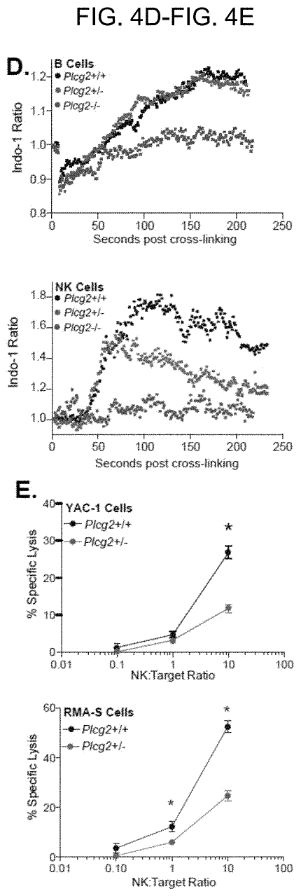

To establish that PLCG2 haploinsufficiency is sufficient to cause NKD, a mouse model of haploinsufficiency was validated by comparing wild type and PIcg2+/− mice. While PIcg2−/− mice have been previously described with severe B cell and NK cell defects, defects in PIcg2+/− mice have not been previously reported. Subpopulation analysis was performed using flow cytometry and viSNE. As expected, major perturbations were seen in PIcg2−/− mice, including altered B cell development; however, B cell and NK cell development were intact in PIcg2+/− mice (see e.g., FIG. 4A and FIG. 4B). Similar to the patients, NK cell maturation was increased in PIcg2+/− mice (see e.g., FIG. 4C). Calcium flux analysis was performed in both B cells and NK cells. Although IgM-induced calcium flux was normal in PIcg2+/− B cells, NK1.1-induced calcium flux was attenuated in PIcg2+/− NK cells (see e.g., FIG. 4D). Correlating with reduced calcium flux, NK cell killing of YAC-1 and RMA-S target cells was inhibited in PIcg2+/− mice (see e.g., FIG. 4E). Similar to the clinical findings in the patients, PIcg2+/− mice had normal degranulation despite reduced cytotoxicity (data not shown). This combination of enhanced NK cell maturation, decreased NK cell calcium flux, and decreased NK cell killing phenocopies the patients and demonstrates that one-copy loss of PLCG2 is sufficient to cause functional NKD.

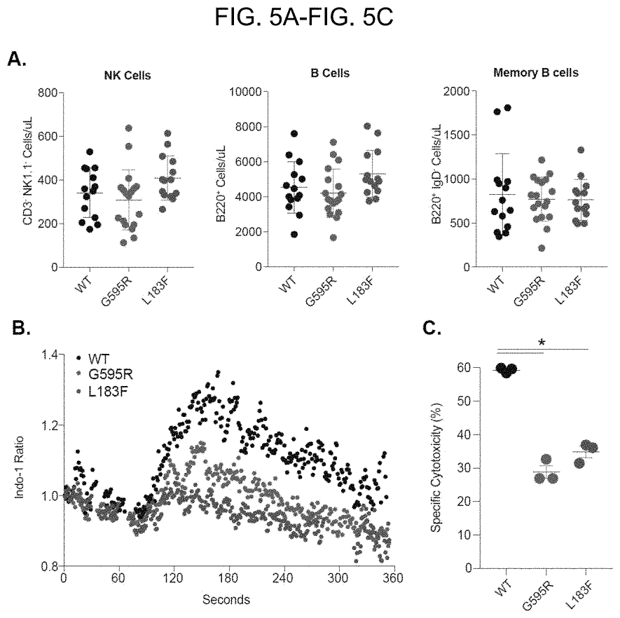

To conclusively link the heterozygous PLCG2 mutations to the phenotypes observed in the patients, CRISPR knock-in mice were generated with heterozygous G595R and L183F mutations. These mice were evaluated for immune phenotype and natural killer cell function. Similar to patients in both families, NK cell counts were preserved (see e.g., FIG. 5A). Despite normal B cell function, family A presented with low B cell counts which was not recapitulated in the G595R CRISPR mice, indicating this likely represents a kindred effect and is separate from the mechanism of disease in PLCG2 haploinsufficiency (see e.g., FIG. 5A). L183F CRISPR mice also demonstrated normal B cell counts, and both G595R and L183F CRISPR mice had normal memory B formation (see e.g., FIG. 5A). NK cell function was tested by examination of NK cell cytotoxicity and calcium flux. Similar to the human patients and haploinsufficient PIcg2 mice, G595R and L183F CRISPR mice had decreased NK1.1-induced calcium flux (see e.g., FIG. 5B) and correspondingly low NK cell cytotoxicity against YAC-1 target cells (see e.g., FIG. 5C). Together, these data demonstrate the G595R and L183F mutations are loss-of-function mutations resulting in functional PLCG2 haploinsufficiency and NK cell functional defects without perturbance of B cell function.

Discussion

The heterozygous loss-of-function mutations presented herein result in PLCG2 haploinsufficiency, NK cell dysfunction, and herpesvirus susceptibility. Despite the role of PLCG2 in B cells, these cells are functionally intact in PLCG2 haploinsufficiency. Based on the differential regulation of PLCG2 expression among lymphocytes, herein is disclosed a threshold model wherein cell types with homeostatically low levels of PLCG2 (i.e., CD56^{Dim} NK cells) are uniquely susceptible to further reductions in PLCG2 function. As a result, most lymphocytes are likely shielded from haploinsufficiency by virtue of their high PLCG2 expression or use of alternative pathways (i.e., PLCG1 in T cells). This model also suggests that PLCG2 may serve as a rate-limiting checkpoint against erroneous cytotoxicity in NK cells, requiring strong PLCG2 activation for accurate and directional degranulation. Extrapolating this model further, monocytopenia was also observed in all patients, and monocytes express the lowest levels of PLCG2 of all cell types examined. While the role of monocytopenia to the observed clinical phenotypes is not clear, this may also contribute to certain features of disease, including herpesvirus and bacterial susceptibility.

Despite a number of similarities (see e.g., TABLE 3), family A and B each possess unique features as well. Notably, B cell output was reduced in family A (including A.II.2), but not family B. This is likely a result from other genetic background effects, as B cell development was unaffected in G595R CRISPR knock-in mice. Families A and B also differed in the nature of their phosphorylation defect. Family A had reduced magnitude of PLCG2 phosphorylation while family B had altered kinetics. An analysis of each domain's function provides insight into this difference. The L183F mutation in family B occurs in the PH domain, which binds P13K-generated PIP₃ at the immunologic synapse and facilitates localization of PLCG2 to the membrane. Upon reaching the membrane, the nSH2 (affected by G595R in family A) binds to phosphorylated LAT, enabling assembly of the NK cell signalosome and interaction of PLCG2 with its kinase (Btk/ltk). Therefore, PLCG2 lacking a functional PH domain would be capable of normal signalosome interaction, but diffusion-limited and kinetically altered. In contrast, PLCG2 lacking a functional nSH2 domain would be blocked from signalosome interaction and phosphorylation altogether, reducing the magnitude of calcium flux. This hypothesis is consistent with the patterns observed in the patients and suggests that PLCG2 loss-of-function mutations may have domain-specific phenotypes.

Pathogenic variants are commonly modifiable by both genetic and environmental factors. Genetic epistasis likely plays a role in the incomplete penetrance and variable expressivity observed in many autosomal dominant syndromes. Immunologic context, such as the cytokine environment, may also modulate cellular and clinical phenotypes. For example, IL-2 incubation partially reverses NK cell killing defects in STX11-deficient patients, reminiscent of the restoration of patient NK cell killing after IL-15/IL-2 cytokine exposure herein (see e.g., FIG. 11C). The use of collateral immunologic pathways may also alter phenotypes. For example, the versatile use of either PLCG1 or PLCG2 by some NK cell receptors (i.e., CD16) may allow compensatory signaling through these pathways. Moreover, the variable nature of the adaptive immune response may compensate for innate defects to different degrees. A combination of these likely contributes to the phenotypic differences and incomplete penetrance seen in PLCG2 haploinsufficiency. This manipulability may also present an opportunity to therapeutically modify defects, for example with modulation of IL-15 signaling using ALT-803, an investigational drug previously shown to rectify NK cell cytotoxicity defects in vivo and aid CMV clearance in humans.

Whereas PLAID and APLAID represent the autosomal dominant manifestations of dominant-negative and gain-of-function mutations, the present patients illustrate haploinsufficiency and expand the spectrum of PLCG2-related disease. A fourth possibility, autosomal recessive loss-of-function, remains either undiscovered or is incompatible with life. While these three syndromes may be mechanistically distinct, there remains unexplained overlap (i.e., autoimmunity) that merits further investigation. Nonetheless, PLCG2 haploinsufficiency results in clinical phenotypes distinct from PLAID/APLAID and requires a different diagnostic and therapeutic approach. PLAID, APLAID and PLCG2 haploinsufficiency are compared in TABLE 4.

TABLE 4
Clinical characteristics of PLAID, APLAID and PLCG2
haploinsufficiency. NR, not reported. PLAID, PLCG2-
associated Antibody Deficiency and Immune Dysregulation.
APLAID, Autoinflammation & PLCG2-associated Antibody
Deficiency and Immune Dysregulation.

| | | | PLCG2 |
|---|---|---|---|
| | PLAID | APLAID | Haploinsufficiency |
| Mutation | cSH2 Deletions | S707Y | G595R, L183F |
| Mechanism of | Dominant- | Gain-of- | Loss-of-function |
| Disease | negative or | function | (haploinsufficiency) |
| | Gain-of-function* | | |
| Cold Urticaria | + | − | − |
| Inflammatory | + | + | − |
| Skin Lesions | | | |
| and Cutaneous | | | |
| Granulomas | | | |
| Allergic | + | − | − |
| Disease | | | |
| Arthralgias | − | + | + |
| Autoantibodies/ | + | − | + |
| Auto immunity | | | |
| Bacterial | + | + | + |
| Susceptibility | | | |
| Herpesvirus | − | − | + |
| Susceptibility | | | |
| NK Cell Count | Decreased | Normal | Normal |
| NK Cell Killing | NR | NR | Decreased |
| NK Cell | Decreased | NR | Normal |
| Degranulation | | | |
| NK Cell Calcium | Decreased | NR | Decreased |
| Influx | | | |
| B Cell Count | Decreased | NR | Normal/ |
| | | | Decreased |
| B Cell Class | Decreased | Decreased | Normal |
| Switching | | | |
| IgG/IgA/IgM | Decreased | Normal | Normal |
| levels | | | |
| B Cell Calcium | Decreased/ | Increased | Normal |
| Influx | Increased^{†} | | |
| Mast Cell | Normal/ | NR | NR |
| Degranulation | Increased^{†} | | |

*Temperature dependent mechanism.
†At 37° C., B cell calcium is inhibited while mast cell degranulation is unchanged. At subphysiologic temperatures B cell calcium and mast cell degranulation are increased.

At present, the lack of a clear etiology complicates the management of many patients with unusually severe and/or recurrent herpesvirus infections. This study highlights a potential role for PLCG2 mutations in these patients, provides insight into the regulation of human NK cell cytotoxicity, and unifies PLCG2-associated disease along a clinical spectrum that now includes PLAID, APLAID, and PLCG2 haploinsufficiency. Unlike PLAID and APLAID which require deletions or mutations at specific locations, loss-of-function mutations could plausibly occur in many domains beyond the SH2 and PH domains, including the catalytic, SH3 and C2 domains. Of the 60,000 healthy exomes in ExAC, only 402 of 1265 residues in PLCG2 have been reported with missense variants. This evolutionary pressure against mutations in PLCG2 implicates a number of residues where variants may disrupt PLCG2 function. As a result, heterozygous PLCG2 mutations should be considered in the differential diagnosis of patients with a number of presentations beyond cold urticaria, antibody deficiency, and autoinflammation, including but not limited to NK cell immunodeficiency and herpesvirus susceptibility

### Exmple 2: Dysregulated NK Cell PLCG2 Signaling and Activity in Juvenile Dermatomyotosis

This example describes how dysregulated PLCG2 phosphorylation and decreased calcium flux in natural killer cells plays a role in juvenile dermatomyotosis (JDM).

Abstract

Juvenile dermatomyositis (JDM) is a debilitating pediatric autoimmune disease manifesting with characteristic rash and muscle weakness. To delineate signaling abnormalities in JDM, mass cytometry was performed with PBMCs from treatment-naive JDM patients and controls. NK cell percentages were lower while frequencies of naive B cells and naïve CD4+T cells were higher in JDM patients than in controls. These cell frequency differences were attenuated with cessation of active disease. A large number of signaling differences were identified in treatment-naive JDM patients compared with controls. Classification models incorporating feature selection demonstrated that differences in phospholipase Cγ2 (PLCG2) phosphorylation comprised 10 of 12 features (i.e., phosphoprotein in a specific immune cell subset) distinguishing the 2 groups.

Because NK cells represented 5 of these 12 features, further studies focused on the PLCG2 pathway in NK cells, which is responsible for stimulating calcium flux and cytotoxic granule movement. No differences were detected in upstream signaling or total PLCG2 protein levels. Hypophosphorylation of PLCG2 and downstream mitogen-activated protein kinase-activated protein kinase 2 were partially attenuated with cessation of active disease. PLCG2 hypophosphorylation in treatment-naive JDM patients resulted in decreased calcium flux. The identification of dysregulation of PLCG2 phosphorylation and decreased calcium flux in NK cells provides potential mechanistic insight into JDM pathogenesis.

Introduction

Juvenile dermatomyositis (JDM) is an inflammatory myopathy/vasculopathy that results in inflammation of striated muscle, skin, and the gastrointestinal tract. It presents with characteristic skin findings (including heliotrope rash, Gottron's papules, and periungual erythema and telangiectasias) and proximal muscle weakness in childhood, with an incidence of 2-3 cases per million children. Before the advent of steroid therapy, JDM had a mortality rate of 40%. Even with treatment, the disease inflicts significant morbidity on children, with over 25% of JDM patients experiencing persistent symptoms for over 36 months and approximately 20% of patients experiencing an even more protracted, refractory disease course.

The etiology of JDM is not well characterized, but both adaptive and innate immune responses have been associated with JDM pathogenesis. Myositis-specific and myositis-associated antibodies (against extractable nuclear antigens) have been identified in approximately 65% of JDM patients.

Furthermore, B cell depletion with rituximab (a chimeric monoclonal antibody against CD20) leads to clinical improvement in some JDM patients. T cells are implicated by the association of JDM with HLA-B08 and HLA-DRB1. Furthermore, JDM patients exhibit increased skewing toward CXCR5+Th2 and Th17 T cell subsets, which correlates with disease activity and blood plasmablasts. The innate immune system also appears to play a role in JDM. Plasmacytoid dendritic cells and macrophage-secreted proteins are present in inflamed JDM patient muscles, and chemokines eotaxin, monocyte chemoattractant protein-1, and IFN-γ-induced protein 10 are elevated in JDM patient serum in comparison with healthy controls. In addition, specific TNF and IL-1 alleles as well as a type I IFN-stimulated gene signature are associated with JDM disease risk.

Several studies have implicated NK cells in the pathogenesis of JDM. NK cells are innate lymphocytes (defined as CD3−CD56+) with germline-encoded receptors that play a critical role in antiviral defense and tumor surveillance. NK cells perform this critical function by secreting immunomodulatory cytokines and releasing cytotoxic granules to lyse target cells. The movement of cytotoxic granules within NK cells is regulated by the phosphorylation of phospholipase Cγ2 (PLCG2) and subsequent generation of calcium flux. There is accumulating evidence that human NK cells play an immunoregulatory role and that NK cell dysfunction may contribute to the onset of human autoimmunity. Despite some experimental limitations, several previous JDM studies have reported evidence of decreased NK cell percentages in the blood of treatment-naive patients compared with controls, a weak association between increased NK cell percentages in the blood and decreased JDM disease activity, and data suggestive of NK cells infiltrating the affected muscle in JDM patients.

Furthermore, decreased NK cell cytotoxicity in JDM has been reported in a small cohort of 5 JDM patients and in 2 additional treatment-naive JDM patients.

Despite these insights, the etiology of JDM is not well understood, and the dysregulation of immune cell signaling in JDM has not been systematically investigated. Therefore, to delineate potential immune cell signaling abnormalities in JDM, mass cytometry was performed on PBMCs from treatment-naive JDM patients and controls. By pairing the deep profiling facilitated by mass cytometry with phospho-specific antibodies, the activation state of 14 signaling molecules in 23 distinct leukocyte subsets was probed within single-patient samples, both at baseline and over a time course following stimulation with a cocktail of cytokines and cross-linking antibodies. This approach identified dysregulated PLCG2 phosphorylation in several immune cell types, with defective PLCG2 phosphorylation in NK cells comprising the primary signaling difference between treatment-naive JDM patients and controls.

Methods

Patients

JDM was defined according to modified Bohan and Peter's criteria. JDM patients diagnosed in pediatric rheumatology clinics at St. Louis Children's Hospital (site 1) or Ann & Robert H. Lurie Children's Hospital of Chicago (site 2) were eligible for enrollment if their cases were new onset and treatment naive. The definition of clinically inactive disease varied slightly between the two sites. Site 1 defined clinically inactive disease as no proximal muscle weakness, no difficulty swallowing, and only residual Gottron's papules or rash. Site 2 defined apparently inactive disease as a disease activity score of 2 or less.

Reagents

Antibodies conjugated to heavy metals were purchased from Fluidigm, with the exception of CD69 (see e.g., TABLE 5).

TABLE 5
Surface and intracellular antibodies for mass cytometry.

| Antigen | Elemental | Clone |
|---|---|---|
| CD45 | 089Y | HI30 |
| CCR6 | 141Pr | G034E3 |
| CD19 | 142Nd | HIB19 |
| CD45RA | 143Nd | HI100 |
| p-PLCγ2 | 144Nd | K86-689.37 |
| CD4 | 145Nd | RPA-T4 |
| IgD | 146Nd | IA6-2 |
| CD11c | 147Sm | Bu15 |
| CD14 | 148Nd | RMO52 |
| CD127 | 149Sm | A019D5 |
| p-STAT5 | 150Nd | 47 |
| CD123 | 151Eu | 6H6 |
| p-AKT | 152Sm | D9E |
| p-STAT1 | 153Eu | 58D6 |
| p-Itk/Btk | 154Sm | 24a/BTK |
| CD27 | 155Gd | L128 |
| CXCR3 | 156Gd | G025H7 |
| p-STAT3 | 158Gd | 4/P-Stat3 |
| p-MAPKAPK2 | 159Tb | 27B7 |
| CD69 | 160Gd | FN50 |
| Ki-67 | 161Dy | B56 |
| p-LCK | 162Dy | 4/LCK-Y505 |
| p-JAK2¹ | 163Dy | D4A8 |
| IkBa | 164Dy | L35A5 |
| CD45RO | 165Ho | UCHL1 |
| p-NFKB | 166Er | K10-95.12.50 |
| p-ERK | 167Er | D13.14.4E |
| CD8 | 168Er | SK1 |
| CD25 | 169Tm | 2A3 |
| CD3 | 170Er | UCHT1 |
| p-Syk/ZAP70 | 171Yb | 17a |
| IgM | 172Yb | MHM-88 |
| HLA-DR | 173Yb | L243 |
| p-STAT4 | 174Yb | 38/p-Stat4 |
| PD-1 | 175Lu | EH12.2H7 |
| CD56 | 176Yb | NCAM16.2 |
| CD16 | 209Bi | 3G8 |

¹pJAK2 was not included in some specimens due to issues with reagent availability.

Sample Preparation and Collection

Blood samples were collected from 17 treatment-naive, new-onset JDM patients, 11 of these 17 JDM patients after achieving clinically inactive disease, and 17 healthy controls (Table 1), and PBMCs were isolated using a Ficoll-Paque PLUS gradient (GE Healthcare) and cryopreserved.

Mass cytometry

PBMCs were thawed and labeled with cisplatin to distinguish live cells (Fluidigm). Cells were aliquoted (1.7×10⁶ to 3.3×10⁶ cells per tube) into polypropylene tubes with 80 μl volume. Cells were stained with all surface marker antibodies except CD45, CD45RA, and CD45RO for 30 minutes at 37° C., washed with warm media, and rested for 30 minutes at 37° C. before stimulation. To maximize insights gained from limited samples, a combination of stimuli to activate different signaling pathways was chosen to stimulate the samples. Cells were left unstimulated or stimulated with 500 U/m1 IL-2 (R&D Systems), 50 ng/ml

IL-12 (R&D Systems), 500 ng/ml LPS (InvivoGen), 500 U/m1 IFN-α4 (PBL InterferonSource), and 1 μl/ml anti-mouse IgG (BioLegend) for 3 or 15 minutes in RPMI1640 medium (Sigma-Aldrich) supplemented with 10% fetal calf serum at 37° C., then fixed with MaxPar Fix I Buffer, permeabilized with MaxPar Barcode Perm Buffer, and barcoded with the Cell-ID 20-Plex Pd Barcoding Kit (Fluidigm). Barcoded samples were pooled and stained with antibodies for CD45, CD45RA, and CD45RO (see e.g., TABLE 5). After staining for surface markers, the pooled samples were methanol permeabilized and stained with antibodies for intracellular markers (see e.g., TABLE 5). Samples were put in Cell-ID Intercalator-Ir (Fluidigm) overnight to facilitate detection of debris and doublets and then run on a CyTOF2/Helios instrument (Fluidigm). Samples were debarcoded using the Single Cell Debarcoder, a standalone MATLAB application. Data were analyzed using Cytobank and R. A run control from the same normal donor was used in each experiment to normalize the phosphoprotein data as follows:

Data normalization: arcsinh(xₛₐₘₚₗₑ/5)−arcsinh(x_{run control}/5)

Citrus

Citrus, a computational technique combining hierarchical clustering with an analysis of stratifying differences in cluster features (i.e., phosphorylation signaling proteins in immune subsets) between 2 groups of samples, was performed with the R Citrus package on flow cytometry standard files gated on live immune cells to compare treatment-naive patients with healthy controls for each stimulation timepoint. Surface markers were clustering parameters. Minimum cluster size was set as 2% of the total population, with 10,000 events sampled per file. Cluster characterization features were signaling molecules. All clustering and characterization features were arcsinh transformed. Differences in cluster features were calculated using LASSO feature selection because the number of stratifying features with SAM was too large to manually interpret. LASSO model cross-validation error rates were acceptably low for model interpretation (see e.g., FIG. 19A-FIG. 19C).

To aid in interpretation of cluster cell type, all surface marker-transformed medians (see e.g., TABLE 5) were visualized in a heat map. A PLS-DA model to classify treatment-naive patients from controls was constructed with LASSO-selected features in Citrus, combining all 3 stimulation time points into a single Z score-transformed matrix for analysis.

Flow cytometry to assess total PLCG2 and SHIP1 protein levels in NK cells

Flow cytometry was performed on a subset of 3 treatment-naive patients (for which samples were available) and 3 matched controls collected at the host site to assess total PLCG2 and SHIP1 levels. Samples (2×10⁵ cells per sample) were stained with surface marker antibodies and fixed with Cytofix/Cytoperm buffer (BD Biosciences). Samples were stained with CD16 (3G8) V500, CD3 (UCHT1) PerCP-Cy5.5, CD19 (SJ25C1) BV786, and total PLCG2 (K86-1161) PE (BD Biosciences), as well as CD56 Pacific Blue (NCAM1), CD45RA (HI100) PE-Cy7, and SHIP1 (P1C1-A5) AF647 (BioLegend). Flow cytometry was performed on a 12-color LRSFortessa X-20 flow cytometer (BD Biosciences) and analyzed with FlowJo (FlowJo, LLC). NK cells were gated as CD56+CD3− lymphocytes and analyzed for differences in PLCG2 and SHIP1 between patient and control samples.

Analysis of NK cell calcium flux via flow cytometry

To assess if differences in NK cell PLCG2 phosphorylation led to functional alterations, flow cytometry-based calcium flux assays were performed on 2 treatment-naive patients and a control sample. NK cells were enriched using an EasySep Human NK Cell Isolation Kit (STEMCELL Technologies) (>86% purity), then loaded with Indo-1 dye (Invitrogen), and labeled with mouse IgG antibodies against the NK cell receptors 2B4 (clone C1.7, BioLegend) and NKG2D (clone 1D11, BD Biosciences). Kinetic measurements of calcium flux were obtained using a BD LRSFortessa X-20 flow cytometer at baseline and then upon antibody cross-linking using anti-mouse IgG.

Statistics

An a value of 0.05 was set to determine significance, incorporating multiple hypothesis correction as appropriate. Error bars in figures represent the mean plus or minus the SEM. Differences in immune cell proportions were assessed by 1-way ANOVA with Bonferroni's correction for multiple comparisons. Signaling differences in canonically gated cell types were confirmed with 2-tailed Welch's t tests with a Bonferroni adjustment to account for testing 897 hypotheses (3 time points with 299 signals in different cell types per each time point). Differences in PLCG2, ltk/Btk, Syk/ZAP70, and MAPKAPK2 signaling time courses between treatment-naive patients and controls were compared using a 2-tailed Welch's t test with Benjamini-Hochberg multiple hypothesis correction (n=3 time points×4 signaling molecules=12 hypotheses). Differences in NK cell activation and proliferation (assessed by CD69 and Ki-67, respectively) between treatment-naive patients and controls were assessed using 2-tailed Student's t tests with Benjamini-Hochberg multiple hypothesis correction. PLCG2 flow cytometry panel data were analyzed using 2-detailed Student's t tests with Benjamini-Hochberg multiple hypothesis correction to account for testing 2 hypotheses.

Study approval

The study was approved by the institutional review boards at Washington University School of Medicine, St. Louis (IRB ID 201109216), and at Ann & Robert H. Lurie Children's Hospital of Chicago (IRB ID 2008-13457 and 2001-11715), and written informed consent was received to use patient samples.

Results

Patient cohort

Samples from 17 treatment-naive JDM patients, 11 of these 17 JDM patients after achieving clinically inactive disease, and 17 healthy controls were analyzed (see e.g., TABLE 6).

TABLE 6
Patient demographics.

| | | | | Age at | Age at clinically | | | | | |
|---|---|---|---|---|---|---|---|---|---|---|
| | | | Duration of | sample | inactive disease | Medications in | | | | |
| | | | untreated disease | collection | sample collection | clinically inactive | | | | |
| Patient | Sex | Race | (months) | (yrs) | (yrs) | dis. sample | MSA | MAA | Healthy control | Site |
| 1 | F | W | 2 | 7.2 | 9.3 | MTX | anti-SAE | negative | 11.2 yr W F | 1 |
| 2 | M | W | 3 | [symbol] .2 | 5.1 | MTX, IVIG | negative | negative | 2.6 yr W M | 1 |
| 3 | M | W | 1 | [symbol] | 10 | MTX, IVIG, PLQ | p155/140 | negative | 7.5 yr W M | 1 |
| 4 | F | W | 12 | 16.3 | | | p155/140 | negative | 13.4 yr W F | 1 |
| 5 | F | W | <1 | 12.8 | | | MJ | negative | 12.8 yr W F | 1 |
| 6 | F | W | <3 | 8.2 | | | p155/140 | negative | 11.2 yr B F | 1 |
| 7 | M | W | 4 | 12.1 | | | MDAS | negative | 15. [symbol] yr B M | 1 |
| 8 | F | W | [symbol] | 4.2 | 8.2 | | p155/140 | negative | 4.1 yr W M | 2 |
| 9 | M | H | 19 | 3 | | | p155/140 | negative | 6.5 yr W M | 2 |
| 10 | F | W | <4 | 9.7 | 14.5 | | Mi-2 | negative | 9.3 yr W M | 2 |
| 11 | F | B | [symbol] | 8.2 | 12. [symbol] | | negative | negative | 11 yr NA/H F | 2 |
| 12 | F | W | 3 | [symbol] .5 | 7.3 | PO, MTX, IVIG, | p155/140 | Ro | 7.1 yr W F | 2 |
| | | | | | | MMF | | | | |
| 13 | F | W/A | 2 | [symbol] .2 | 7.1 | | negative | negative | 8.8 yr W F | 2 |
| 14 | F | B | 2 | 6.1 | 15. [symbol] | MTX, CSA, MMF | Mi-2, p155/140 | negative | 11 yr W F | 2 |
| 15 | F | W | 13 | [symbol] .6 | 16.0 | | p155/140 | Ro | 13 yr W F | 2 |
| 16 | F | W | 8 | [symbol] .2 | 15.0 | | p155/140 | negative | 6 yr NA/H F | 2 |
| 17 | F | W | [symbol] | 2.3 | | | negative | negative | 7 yr W F | 2 |

Research site 1 denotes St. Louis Children's Hospital, and research site 2 denotes Lurie Children's Hospital.
If not noted, patient was not on medication at the time of clinically inactive disease sample collection.
Medication abbreviations: MSA, myositis-specific autoantibody; MAA, myositis-associated autoantibody; SAE, small ubiquitin-like modifier (SUMO) activating enzyme; PO, oral prednisone; MTX, methotrexate; IVIG, intravenous immunoglobin; PLQ, plaquenil; MJ, also known as nuclear matrix protein 2 (NXP-2) CSA, cyclosporine; MMF, mycophenolate mofetil.
Abbreviations for race: W, White; B, African American; H, Hispanic; A, Asian. [symbol]
indicates data missing or illegible when filed

The mean age of the patients and the controls in the cohort were 7.4 years and 9.3 years, respectively, with similar sex distributions (76% and 70.6% girls in patients and controls, respectively). Eighty-two percent of the patients were White (compared with 70.6% of the controls). The median duration of untreated disease in the patients was 3.6 months (average 5.6 months with a standard deviation of 5 months).

Cell percentages

Mass cytometry was used to quantify the distribution of 23 distinct leukocyte subsets in samples from treatment-naive JDM patients, healthy controls, and a subset of the JDM patients after achieving clinically inactive disease. Samples were gated on live immune cell singlets and then into 23 immune cell types, based on distribution of surface markers (see e.g., FIG. 18A and FIG. 18B). NK cells were present at a lower frequency while the percentages of naive B cells and naive CD4+ T cells were higher in treatment-subsets naive JDM patients than in controls (see e.g., FIG. 13A). Frequency of PBMC subsets was also examined in 11 paired treatment-naive and clinically inactive JDM patient samples. Naive B cell frequency normalized in paired samples with cessation of active disease (see e.g., FIG. 13B). Although there was a trend toward increased NK cell percentages with cessation of active disease in paired samples (with increased NK cell percentages in 9 of the 11 paired samples), the difference was not statistically significant after multiple hypothesis correction (see e.g., FIG. 13B; t=2.37, degrees of freedom [df]=10, P=0.039). However, there was no statistically significant difference in NK cell percentages between the samples from JDM patients with clinically inactive disease and healthy controls (mean ±standard deviation of 6.00±2.89 and 7.60±5.42 for the JDM patients with clinically inactive disease and healthy controls, respectively; t =1.04, df=26, P=0.310), supporting the trend toward normalization in NK cell percentages with cessation of active disease.

Signaling phenotype

Differences in signaling between treatment-naive JDM patients and controls (or patients with clinically inactive disease) were also examined. To simultaneously gain insights about multiple signaling pathways, samples were stimulated concurrently with IL-2, IL-12, LPS, and IFN-α4 as well as IgM, CD3, and CD16 cross-linking for 0, 3, or 15 minutes and then subjected to mass cytometry to quantify phosphorylation of a panel of 14 intracellular signaling molecules (see e.g., TABLE 5). Because 292 stratifying (i.e., distinguishing) features were detected when significance analysis of microarrays (SAM) was used to compare JDM patients and controls (data not shown), a method incorporating feature selection was necessary to aid in interpreting the results.

Feature selection techniques, such as least absolute shrinkage and selection operator (LASSO), enhance generalization by reducing overfitting and removing redundant or irrelevant features (e.g., features that are redundant in the presence of another correlated feature). Cluster identification, characterization, and regression (Citrus), a technique that combines unsupervised hierarchical clustering with a regularized supervised learning algorithm to predict the class of the samples (e.g., patients versus controls) from the features of a data set (e.g., phosphorylation of a signaling molecule in an immune subset/cluster), with LASSO regression was used to determine which features were stratifying between treatment-naive JDM patients and controls. This approach identified NK cell subsets as stratifying for each stimulation time point as well as unstimulated classical monocytes and T cells (see e.g., FIG. 14A). The 12 stratifying features Citrus identified (unstimulated as well as 3- and 15-minute-stimulated p-PLCG2 in NK cell clusters, unstimulated p-STAT3 in a subset of NK cells, unstimulated p-PLCG2 in a classical monocyte subset, unstimulated as well as 3- and 15-minute-stimulated p-PLCG2 in CD4+and CD8+T cell clusters, and 3-minute-stimulated p-STAT3 in nonclassical monocytes) were sufficient to completely segregate treatment-naive JDM patient samples from control samples by hierarchical clustering (see e.g., FIG. 14B).

A partial least squares discriminant analysis (PLS-DA) model was constructed from the selected features to visualize the stratifying signaling features in relation to classification as patient or control (see e.g., FIG. 14C and FIG. 14D). PLS-DA was used to decompose matrices of signaling data and disease state into scores and loadings matrices, with the hypothesis that the classification of a sample as a treatment-naive patient or control is dependent upon the signaling profile of the sample. The scores plot describes the relationship of the samples to one another (see e.g., FIG. 14C), and the loadings plot describes the relationships of the variables (signaling protein phosphorylation in specific immune cell clusters) to one another (see e.g., FIG. 14D). The PLS-DA model was able to completely distinguish patients and controls (see e.g., FIG. 14C). Furthermore, the loadings plot demonstrated that treatment-naive JDM patient samples were associated with lower levels of NK cell p-PLCG2 for all stimulation time points (and unstimulated classical monocyte p-PLCG2) in comparison with control samples, while p-PLCG2 in stratifying T cell clusters and p-STAT3 in NK cell and nonclassical monocyte clusters were higher in treatment-naive JDM patient samples than in controls (see e.g., FIG. 14D). PLCG2 dysregulation was also detected in bulk (manually gated) immune cell populations corresponding to the observation in the immune cell subsets represented by the Citrus clusters (see e.g., FIG. 23A-FIG. 23D and FIG. 24).

Given that many of the detected stratifying differences were in NK cells (5 of 12 Citrus features) and PLCG2 (10 of 12 Citrus features) (see e.g., FIG. 2D), the significance of NK cell PLCG2 phosphorylation was confirmed using 2-tailed Welch's t tests with stringent Bonferroni correction to account for 897 comparisons (299 features examined for each of the 3 time points). This statistical test specified that the 3 most significant features (phosphoprotein in a specific immune cell subset) were NK cell p-PLCG2 at 0, 3, and 15 minutes (see e.g., TABLE 7) and that 7 of the 9 features involved p-PLCG2 and 1 involved phosphorylated MAPK-activated protein kinase 2 (p-MAPKAPK2), a downstream kinase in the PLCG2 signaling cascade (see e.g., TABLE 7), clearly highlighting the importance of dysregulated NK cell p-PLCG2 in JDM. Therefore, subsequent studies focused on NK cell PLCG2 signaling.

TABLE 7
Significant p-values using 2 tailed Welch's t-test and MHC.

| Name | tval | pval | FDRthresh |
|---|---|---|---|
| NK cells | −7.852986436 | 2.42584E−08 | 5.57414E−05 |
| p-PLCγ2 unst* | | | |
| NK cells | −7.498464205 | 1.50352E−07 | 0.000111483 |
| p-PLCγ2 15 min* | | | |
| NK cells | −7.260336613 | 1.81878E−07 | 0.000167224 |
| p-PLCγ2 3 min* | | | |
| Non-classical | −6.475463858 | 3.28929E−07 | 0.000222965 |
| monocytes | | | |
| p-PLCγ2 unst* | | | |
| Non-classical | −5.907332717 | 1.76436E−06 | 0.000278707 |
| monocytes | | | |
| p-PLCγ2 3 min* | | | |
| mDCs p-PLCγ2 | −5.534029597 | 4.55974E−06 | 0.000334448 |
| unst* | | | |
| Non-classical | −5.093395629 | 1.55108E−05 | 0.00039019 |
| monocytes | | | |
| pMAPKAPK2 | | | |
| 15 min* | | | |
| Non-classical | −4.931659373 | 2.50224E−05 | 0.000445931 |
| monocytes | | | |
| p-PLCγ2 15 min* | | | |
| Classical | −4.771229485 | 3.86315E−05 | 0.000501672 |
| monocytes | | | |
| p-ERK 15 min* | | | |
| Naive B cells | −4.581093204 | 0.000114761 | 0.000557414 |
| p-STAT3 unst | | | |
| Non-classical | −4.402050767 | 0.000115162 | 0.000613155 |
| monocytes | | | |
| p-NFkB 15 min | | | |
| Unswitched | −4.60841832 | 0.000134452 | 0.000668896 |
| memory | | | |
| B cells p-PLCγ2 | | | |
| 15 min | | | |
| Naive B cells | −4.324920957 | 0.000140748 | 0.000724638 |
| p-AKT 15 min | | | |
| Naive B cells | −4.465519349 | 0.000155212 | 0.000780379 |
| p-PLCγ2 unst | | | |
| Classical | −4.385934405 | 0.000182175 | 0.00083612 |
| monocytes | | | |
| p-NFkB 15 min | | | |
| Naive B cells | −4.18625037 | 0.000216521 | 0.000891862 |
| p-AKT 3 min | | | |
| Non-classical | −4.319755417 | 0.000216603 | 0.000947603 |
| monocytes | | | |
| p-AKT 15 min | | | |
| Classical | −4.175200378 | 0.000216783 | 0.001003344 |
| monocytes | | | |
| p-PLCγ2 3 min | | | |
| Naive B cells | −4.165542806 | 0.00022982 | 0.001059086 |
| p-AKT | | | |
| unst | | | |
| Classical | −4.216688736 | 0.000242932 | 0.001114827 |
| monocytes | | | |
| IkBa unst | | | |
| NK cells | −4.432499128 | 0.000246073 | 0.001170569 |
| pMAPKAPK2 | | | |
| 15 min | | | |
| Classical | −4.221088834 | 0.000261133 | 0.00122631 |
| monocytes | | | |
| p-AKT 15 min | | | |
| Classical | −4.128551449 | 0.000291522 | 0.001282051 |
| monocytes | | | |
| IkBa 3 min | | | |
| Classical | −4.051880176 | 0.00030337 | 0.001337793 |
| monocytes | | | |
| p-PLCγ2 unst | | | |
| Classical | −4.017485092 | 0.000401034 | 0.001393534 |
| monocytes | | | |
| p-AKT 3 min | | | |
| Classical | −4.052172809 | 0.000413522 | 0.001449275 |
| monocytes | | | |
| p-ZAP70/SYK | | | |
| unst | | | |
| Class-switched | −4.021879424 | 0.000429642 | 0.001505017 |
| memory B cells | | | |
| p-STAT3 unst | | | |
| NK cells | −4.177410213 | 0.000446839 | 0.001560758 |
| pMAPKAPK2 | | | |
| 3 min | | | |
| Classical | −3.91696179 | 0.000463162 | 0.001616499 |
| monocytes | | | |
| p-STAT5 3 min | | | |
| IgM memory B | −4.081576067 | 0.000497738 | 0.001672241 |
| cells | | | |
| p-PLCγ2 unst | | | |
| CD8 CM T cells | −3.898795411 | 0.000532513 | 0.001727982 |
| p-AKT 15 min | | | |
| mDCs p-AKT | −4.00689282 | 0.000553302 | 0.001783724 |
| 15 min | | | |
| Classical | −3.869090839 | 0.000581397 | 0.001839465 |
| monocytes | | | |
| p-STAT5 unst | | | |
| pDCs p-PLCγ2 | −4.033481198 | 0.000608125 | 0.001895206 |
| 15 min | | | |
| IgM memory | −4.031382903 | 0.000643085 | 0.001950948 |
| B cells | | | |
| p-PLCγ2 15 min | | | |
| CD8 CM T cells | −3.848868811 | 0.000664293 | 0.002006689 |
| p-AKT 3 min | | | |
| mDCs p-PLCγ2 | −3.727960689 | 0.00075426 | 0.00206243 |
| 15 min | | | |
| Non-classical | −3.790249617 | 0.00077987 | 0.002118172 |
| monocytes | | | |
| p-AKT 3 min | | | |
| Non-classical | −3.713451586 | 0.000806289 | 0.002173913 |
| monocytes | | | |
| p-STAT1 3 min | | | |
| Non-classical | −3.772895964 | 0.000843054 | 0.002229654 |
| monocytes | | | |
| pMAPKAPK2 3 min | | | |
| Non-classical | −3.693105014 | 0.000893942 | 0.002285396 |
| monocytes | | | |
| p-ERK 15 min | | | |
| NK cells | −3.824242602 | 0.000897015 | 0.002341137 |
| pMAPKAPK2 unst | | | |
| Classical | −3.725288925 | 0.00094697 | 0.002396878 |
| monocytes | | | |
| p-AKT unst | | | |
| IgM memory | −3.859116855 | 0.001001104 | 0.00245262 |
| B cells | | | |
| p-PLCγ2 3 min | | | |
| pDCs p-PLCγ2 | −3.751242004 | 0.001023663 | 0.002508361 |
| unst | | | |
| mDCs p-PLCγ2 | −3.570848826 | 0.001149672 | 0.002564103 |
| 3 min | | | |
| Naive B cells | −3.642749714 | 0.001229433 | 0.002619844 |
| p-PLCγ2 3 min | | | |
| Naive CD8 | −3.675820485 | 0.001271397 | 0.002675585 |
| T cells | | | |
| p-ERK 15 min | | | |
| mDCs p-AKT unst | −3.654776711 | 0.00128198 | 0.002731327 |
| Unswitched memory | −3.743624532 | 0.00129909 | 0.002787068 |
| B cells | | | |
| p-PLCγ2 unst | | | |
| CD8 EM T cells | −3.633263879 | 0.001311013 | 0.002842809 |
| IkBa unst | | | |
| CD8 CM T cells | −3.594127595 | 0.001362115 | 0.002898551 |
| p-AKT unst | | | |
| Non-classical | −3.657639336 | 0.001391583 | 0.002954292 |
| monocytes | | | |
| pMAPKAPK2 unst | | | |
| Non-classical | −3.573470136 | 0.001414836 | 0.003010033 |
| monocytes | | | |
| p-ZAP70/SYK | | | |
| 3 min | | | |
| CD8 CM T cells | −3.603965427 | 0.001436343 | 0.003065775 |
| p-NFkB 3 min | | | |
| Class-switched | −3.508707981 | 0.001445751 | 0.003121516 |
| memory B cells | | | |
| p-AKT unst | | | |
| Class-switched | −3.476047577 | 0.00148754 | 0.003177258 |
| memory B cells | | | |
| p-AKT 3 min | | | |
| CD8 TEMRA p-AKT | −3.503207678 | 0.001498708 | 0.003232999 |
| 15 min | | | |
| Classical | −3.55602452 | 0.001503956 | 0.00328874 |
| monocytes | | | |
| p-NFkB unst | | | |
| Non-classical | −3.600974645 | 0.001597665 | 0.003344482 |
| monocytes | | | |
| p-NFkB | | | |
| unst | | | |
| Classical monocytes | −3.534940025 | 0.001623767 | 0.003400223 |
| p-NFkB 3 min | | | |
| NK cells p-AKT | −3.522490015 | 0.001749898 | 0.003455964 |
| 15 min | | | |
| Naive B cells | −3.463834188 | 0.001795491 | 0.003511706 |
| p-PLCγ2 15 min | | | |
| Class-switched | −3.622862181 | 0.001817119 | 0.003567447 |
| memory B cells | | | |
| p-PLCγ2 unst | | | |
| Non-classical | −3.430234587 | 0.001982258 | 0.003623188 |
| monocytes | | | |
| p-NFkB 3 min | | | |
| TH17/22 p-PLCγ2 | −3.380269726 | 0.002049574 | 0.00367893 |
| 3 min | | | |
| Naive CD8 | −3.388441856 | 0.002100108 | 0.003734671 |
| T cells | | | |
| p-AKT 15 min | | | |
| Classical | −3.343633827 | 0.00219741 | 0.003790412 |
| monocytes | | | |
| pMAPKAPK2 15 min | | | |
| TFH p-AKT unst | −3.3742471 | 0.002297207 | 0.003846154 |
| Naive CD8 | −3.374354435 | 0.002386375 | 0.003901895 |
| T cells | | | |
| p-ERK 3 min | | | |
| mDCs p-AKT | −3.398881571 | 0.002403988 | 0.003957637 |
| 3 min | | | |
| NK cells | −3.387935699 | 0.002427461 | 0.004013378 |
| p-AKT 3 min | | | |
| Class-switched | −3.288670837 | 0.002478457 | 0.004069119 |
| memory B cells | | | |
| p-AKT 15 min | | | |
| CD4 CM p-AKT | −3.336454037 | 0.002524976 | 0.004124861 |
| 3 min | | | |
| CD8 EM T cells | −3.316517198 | 0.002576971 | 0.004180602 |
| IkBa 3 min | | | |
| mDCs p-STAT5 | −3.283645486 | 0.002616175 | 0.004236343 |
| unst | | | |
| NK cells p-STAT3 | 3.332834793 | 0.002716647 | 0.004292085 |
| unst | | | |
| CD4 EM p-PLCγ2 | −3.279326492 | 0.002748886 | 0.004347826 |
| 3 min | | | |
| CD4 CM p-AKT | −3.291346836 | 0.002836356 | 0.004403567 |
| unst | | | |
| IgM memory | −3.231259371 | 0.002856454 | 0.004459309 |
| B cells | | | |
| p-AKT unst | | | |
| Naive CD8 | −3.332624156 | 0.002857043 | 0.00451505 |
| T cells | | | |
| p-ERK unst | | | |
| Unswitched | −3.348006256 | 0.00291983 | 0.004570792 |
| memory B cells | | | |
| p-PLCγ2 3 min | | | |
| Naive CD8 | −3.2931526 | 0.002921574 | 0.004626533 |
| T cells | | | |
| p-PLCγ2 unst | | | |
| pDCs p-PLCγ2 | −3.376797972 | 0.003049141 | 0.004682274 |
| 3 min | | | |
| IgM memory | −3.204208034 | 0.003070744 | 0.004738016 |
| B cells | | | |
| p-AKT 3 min | | | |
| Class-switched | −3.395494296 | 0.003085244 | 0.004793757 |
| memory B cells | | | |
| p-PLCγ2 15 min | | | |
| Naive CD8 | −3.239428869 | 0.003107065 | 0.004849498 |
| T cells | | | |
| p-AKT 3 min | | | |
| pDCs p-NFkB | −3.292382589 | 0.003177485 | 0.00490524 |
| unst | | | |
| CD8 CM T cells | −3.221865275 | 0.003218725 | 0.004960981 |
| p-NFkB 15 min | | | |
| Unswitched | −3.404283166 | 0.003294037 | 0.005016722 |
| memory B cells | | | |
| pMAPKAPK2 | | | |
| 15 min | | | |
| Naive CD8 | −3.262248851 | 0.003343216 | 0.005072464 |
| T cells p-PLCγ2 | | | |
| 15 min | | | |
| Tregs p-AKT | −3.199021407 | 0.003370795 | 0.005128205 |
| 3 min | | | |
| Class-switched | −3.342794512 | 0.003400203 | 0.005183946 |
| memory B cells | | | |
| p-PLCγ2 3 min | | | |
| CD8 CM T cells | −3.295327918 | 0.003463346 | 0.005239688 |
| p-NFkB unst | | | |
| IgM memory | −3.155224622 | 0.003500603 | 0.005295429 |
| B cells | | | |
| p-ZAP70/SYK unst | | | |
| Naive CD8 | −3.244108017 | 0.003511206 | 0.005351171 |
| T cells | | | |
| p-PLCγ2 3 min | | | |
| Naive CD4 | −3.257447901 | 0.003535796 | 0.005406912 |
| T cells | | | |
| p-PLCγ2 3 min | | | |
| CD4 CM | −3.191298426 | 0.00355494 | 0.005462653 |
| p-PLCγ2 3 min | | | |
| Naive CD8 | −3.16759611 | 0.003749432 | 0.005518395 |
| T cells | | | |
| p-STAT4 3 min | | | |
| CD4 CM p-AKT | −3.155078427 | 0.003778482 | 0.005574136 |
| 15 min | | | |
| Naive CD4 | −3.173456488 | 0.003883861 | 0.005629877 |
| T cells | | | |
| p-AKT 3 min | | | |
| Naive CD8 | −3.107778253 | 0.004005701 | 0.005685619 |
| T cells | | | |
| p-LCK 15 min | | | |
| IgM memory | −3.104200254 | 0.004063048 | 0.00574136 |
| B cells | | | |
| p-AKT 15 min | | | |
| CD4 CM IkBa | −3.179637231 | 0.004126611 | 0.005797101 |
| 3 min | | | |
| TH1 p-AKT | −3.133980329 | 0.004170055 | 0.005852843 |
| 3 min | | | |
| CD4 TEMRA | −3.084840822 | 0.004209115 | 0.005908584 |
| p-ZAP70/SYK | | | |
| 3 min | | | |
| CD8 CM T cells | −3.172215067 | 0.004265661 | 0.005964326 |
| IkBa 3 min | | | |
| Class-switched | −3.079361952 | 0.004288836 | 0.006020067 |
| memory B cells | | | |
| p-STAT3 15 min | | | |
| NK cells | −3.136855295 | 0.004559049 | 0.006075808 |
| p-AKT unst | | | |
| CD4 TEMRA | −3.066632579 | 0.004562753 | 0.00613155 |
| pMAPKAPK2 unst | | | |
| CD4 p-ERK 3 min | −3.050840446 | 0.00456971 | 0.006187291 |
| Naive CD8 | −3.161824418 | 0.004774268 | 0.006243032 |
| T cells IkBa | | | |
| 3 min | | | |
| TFH p-AKT 15 min | −3.063074407 | 0.004842181 | 0.006298774 |
| TH2 p-PLCγ2 | −3.083410753 | 0.004848321 | 0.006354515 |
| 3 min | | | |
| TFH p-AKT 3 min | −3.076026225 | 0.004898365 | 0.006410256 |
| CD8 CM T cells | −3.141189278 | 0.004912292 | 0.006465998 |
| IkBa unst | | | |
| Tregs p-AKT unst | −3.041373313 | 0.005080226 | 0.006521739 |
| Non-classical | −3.015180288 | 0.005368756 | 0.00657748 |
| monocytes | | | |
| p-STAT1 | | | |
| 15 min | | | |
| Naive CD8 T cells | −3.015932582 | 0.005409759 | 0.006633222 |
| p-AKT unst | | | |
| CD8 TEMRA p-AKT | −2.985277009 | 0.005494042 | 0.006688963 |
| 3 min | | | |
| CD4 EM p-AKT | −3.056428529 | 0.005533822 | 0.006744705 |
| unst | | | |
| mDCs p-ERK | −3.044660179 | 0.005613143 | 0.006800446 |
| 15 min | | | |
| TH1 p-AKT | −3.000354694 | 0.005903431 | 0.006856187 |
| 15 min | | | |
| TH1 p-AKT unst | −2.978988325 | 0.005996179 | 0.006911929 |
| CD8 CM T cells | −2.998182731 | 0.006206259 | 0.00696767 |
| p-ERK 3 min | | | |
| CD8 EM T cells | −2.936492295 | 0.006240829 | 0.007023411 |
| p-AKT 15 min | | | |
| Tregs p-AKT | −2.958468378 | 0.006240873 | 0.007079153 |
| 15 min | | | |
| TH1 IkBa 3 min | −3.02117059 | 0.00639121 | 0.007134894 |
| NK T cells p | 3.024677793 | 0.006412514 | 0.007190635 |
| ZAP70/SYK 15 min | | | |
| CD4 EM p-AKT | −2.955508607 | 0.006447829 | 0.007246377 |
| 3 min | | | |
| IgM memory B cells | −2.910286202 | 0.006523654 | 0.007302118 |
| p-STAT3 unst | | | |
| CD4 CM IkBa unst | −2.99732679 | 0.006607945 | 0.00735786 |
| Naive CD4 T cells | −2.948283458 | 0.006625331 | 0.007413601 |
| p-PLCγ2 15 min | | | |
| CD8 TEMRA IkBa | −2.979820568 | 0.007080528 | 0.007469342 |
| 3 min | | | |
| Class-switched | −2.913304778 | 0.007088316 | 0.007525084 |
| memory B cells | | | |
| p-STAT3 3 min | | | |
| Naive CD8 T cells | −2.89216144 | 0.007203813 | 0.007580825 |
| p-STAT4 15 min | | | |
| Unswitched memory | −2.868796619 | 0.007238945 | 0.007636566 |
| B cells p-AKT | | | |
| 3 min | | | |
| Naive CD8 T cells | −2.973565525 | 0.007443768 | 0.007692308 |
| IkBa 15 min | | | |
| IgM memory B | −3.013202668 | 0.007472737 | 0.007748049 |
| cells pMAPKAPK2 | | | |
| 15 min | | | |
| TH2 p-AKT 3 min | −2.904080312 | 0.007529999 | 0.00780379 |

*Denotes significant by Bonferroni correction (α = 0.05/897). All others are significant by Benjamini-Hochberg FDR multiple hypothesis correction.

NK cell PLCG2 signaling cascade

The NK cell signaling time course was examined for phosphorylation of PLCG2 as well as of 2 kinases upstream of PLCG2 (spleen tyrosine kinase [Syk]/zeta-chain-associated protein kinase 70 [ZAP70], IL-2-inducible T cell kinase [Itk]/Bruton's tyrosine kinase [Btk]) and a downstream kinase (MAPKAPK2) in the PLCG2 signaling cascade. NK cell PLCG2 phosphorylation was lower in treatment-naive JDM patients than controls for all time points (see e.g., FIG. 15A, manually gated on NK cells). Interestingly, available samples for a subset (n=11) of these JDM patients while in a clinically inactive disease state (note that 5 of the 11 patients with clinically inactive disease were on medications) displayed an intermediate time course between treatment-naive JDM patients and controls (see e.g., FIG. 20A), suggesting that the observed phosphorylation differences are not likely due to germline mutations in PLCG2. No statistically significant differences were observed in the phosphorylation of upstream signaling molecules Syk/ZAP70 or ltk/Btk in NK cells between treatment-naive JDM patients and controls (see e.g., FIG. 15B and FIG. 15C). Phosphorylation of the downstream kinase MAPKAPK2 was lower in treatment-naive JDM patients, similar to what was seen with p-PLCG2 (see e.g., FIG. 15D).

Given that differences were detected in PLCG2 phosphorylation kinetics, several potential mechanisms for this hypophosphorylation were examined. Flow cytometry was performed with available remaining samples from 3 treatment-naive JDM patients and controls to assess NK cell levels of PLCG2 protein as well as total phosphatidylinositol-3,4,5-trisphosphate 5-phosphatase 1 (SHIP1) protein, an inhibitory molecule in the PLCG2 signaling cascade (see e.g., FIG. 16A-FIG. 16C). No significant difference was detected in total protein levels of PLCG2 (see e.g., FIG. 16A), suggesting that PLCG2 hypophosphorylation in JDM patient NK cells was not simply due to lower PLCG2 expression levels. Unexpectedly, SHIP1 protein levels were lower in treatment-naive JDM patients than in controls (see e.g., FIG. 16B).

PLCG2 is a key signaling component downstream of many NK cell receptors, including CD16, 2B4, and NKG2D. CD16 cross-linking in the stimulation cocktail was upstream of the observed phosphorylation of PLCG2 in NK cells; therefore, it was of interest to determine if CD16 receptor expression levels differed between treatment-naive patients and controls. CD16 expression was indeed significantly lower in treatment-naive JDM patients in comparison with controls (see e.g., FIG. 16C). Given the observed variation in treatment-naive patient NK cell CD16 expression, p-PLCG2 integrated over time (i.e., the area under the p-PLCG2 signal-versus-time plot, a metric that captures the duration and magnitude of p-PLCG2 signaling) was plotted versus CD16 expression levels (arcsinh MFI of CD16), demonstrating a significantly positive correlation with the p-PLCG2 integrated time course in treatment-naive patients but not healthy controls or patients with clinically inactive disease (see e.g., FIG. 16D and FIG. 21). Patients with clinically inactive disease displayed an intermediate slope between treatment-naive patients and controls.

The positive correlation of CD16 expression levels with PLCG2 signaling over time in treatment-naive patient NK cells could suggest that lower p-PLCG2 signaling was due to decreased CD16 receptor expression levels; however, the normal phosphorylation of the 2 upstream kinases (Syk/ZAP70 and ltk/Btk) that lie between CD16 and PLCG2 suggests that the lower CD16 levels are correlative but not causative.

Impact of lower NK cell p-PLCG2

As an assessment of the functional consequences of lower p-PLCG2 levels, calcium flux was evaluated by flow cytometry in enriched NK cells from available samples from 2 treatment-naive JDM patients and 1 healthy control. The treatment-naive JDM patients displayed suppressed calcium flux following 2B4 and NKG2D receptor cross-linking in comparison with the healthy control (see e.g., FIG. 17A). Expression levels of 2B4 and NKG2D did not differ between the treatment-naive JDM patients and control (see e.g., FIG. 17B and FIG. 17C), verifying that the diminished calcium flux was not due to decreased NK cell-activating receptor levels. The reduced calcium flux demonstrated that the hypophosphorylation of PLCG2 has functional consequences and strongly suggests that NK cell granule movement and cytotoxicity would be impaired in treatment-naive JDM patients.

Finally, median signaling intensities of CD69 and Ki-67 (normalized to the run control by the arcsinh ratio) were also evaluated in samples as surrogates for cellular activation and proliferation. Treatment-naive JDM patients' NK cells were more activated than control cells, as assessed by median levels of normalized CD69 intensity (see e.g., FIG. 22A). Furthermore, treatment-naive JDM patients' NK cells were actively proliferating more than control NK cells, as demonstrated by normalized median intensity of Ki-67 (see e.g., FIG. 22B).

Discussion

Based on the hypothesis that cell signaling differences (particularly in innate leukocytes) may contribute to early disease in JDM, this study was designed to delineate signaling differences in peripheral immune cell subsets between treatment-naive JDM patients and controls using the high-dimensional capabilities of mass cytometry coupled with phospho-specific antibodies. Many differences were detected; however, using several analysis approaches (including Citrus with LASSO feature selection), signaling differences that were sufficient to differentiate between JDM patients and controls were found to primarily involve the phosphorylation of PLCG2 in NK cells as well as in classical monocytes, CD4+ T cells, and CD8+ T cells. Given (a) the prominence of the decreased NK cell p-PLCG2 in the identified stratifying features (4 of 12) in the Citrus analysis (see e.g., FIG. 14D) and (b) that NK cell PLCG2 phosphorylation comprised the top 3 out of 9 significant differences identified in 897 features using 2-tailed Welch's t tests with stringent Bonferroni correction (see e.g., TABLE 7), hypophosphorylation of PLCG2 in NK cells appears to be the most important signaling difference distinguishing treatment-naive JDM patients from controls. Dysregulation of PLCG2 in JDM does not appear to have been previously reported.

NK cell percentages in the blood were decreased in treatment-naive JDM patients in comparison with controls, with a trend toward normalization with the cessation of active disease. Previous work suggested that NK cells may be enriched in affected muscles of JDM patients with short disease duration at diagnosis in comparison with patients with longer disease duration, raising the possibility that NK cells may migrate from the blood to affected muscles early in the JDM disease course. Despite the paucity of NK cells in the peripheral blood, NK cells from treatment-naive JDM patients were more highly activated and proliferating to a greater extent than NK cells from healthy controls, as assessed by CD69 and Ki-67, respectively. Interestingly, decreased NK cell frequencies observed in treatment-naive JDM patients correlated with lower levels of PLCG2 phosphorylation (see e.g., FIG. 21). In contrast, NK cell frequency was not correlated with PLCG2 phosphorylation in JDM patients with clinically inactive disease or in healthy controls (see e.g., FIG. 21).

Treatment-naive JDM patient NK cells exhibited lower levels of PLCG2 phosphorylation than healthy controls at all stimulation time points. Phosphorylation of the upstream kinases Syk/ZAP70 and ltk/Btk in the PLCG2 signaling cascade was not different between JDM patients and controls, suggesting that PLCG2 hypophosphorylation is due to other factors (e.g., inhibitory molecules), although the dynamic range of Syk/ZAP70 and ltk/Btk signaling necessary for normal phosphorylation of PLCG2 is not well established. Minimal differences were seen in PLCG2 phosphorylation based on the presence of myositis-specific antibodies (see e.g., FIG. 20A-FIG. 20C). Interestingly, the 3 patients with no myositis-specific antibodies had the lowest levels of p-PLCG2, although there was not enough statistical power to detect a significant difference.

PLCG2 hypophosphorylation after receptor cross-linking resulted in substantially suppressed calcium flux in treatment-naive patients compared with controls. Cross-linking of 2B4 and NKG2D receptors leads to synergistic, selective PLCG2 phosphorylation and calcium mobilization. No differences were observed in expression levels of 2B4 and NKG2D receptors between treatment-naive JDM patients and controls. PLCG2 phosphorylation results in a conformational change in PLCG2, facilitating the hydrolysis of the membrane phospholipid phosphatidylinositol 4,5-bisphosphate to inositol triphosphate (IP3) and diacylglycerol. IP3 subsequently binds to its receptor on the endoplasmic reticulum and releases cellular stores of calcium. Decreased calcium flux is associated with altered cytotoxic granule movement and localization to the immune synapse, resulting in poor NK cell-mediated killing. Therefore, the PLCG2 hypophosphorylation and decreased calcium flux observed in the JDM patients suggests that NK cells from treatment-naive JDM patients would have decreased NK cell cytotoxicity. This is supported by prior observations of decreased NK cell cytotoxicity in small cohort of 5 JDM patients and a second small study with 5 untreated patients with dermatomyositis (2 of whom were adolescents with JDM) who were found to have low NK cell cytotoxicity compared with controls.

After the analysis was completed, it was determined that patient 7 had given informed consent during his initial hospitalization, but his study blood sample was not drawn until his first clinic visit 5 weeks later. He was inadvertently included in the analysis as a treatment-naive patient despite having received methylprednisolone (2 mg/kg i.v. for 3 days) followed by oral prednisolone (0.8 mg/kg titrated down to 0.4 mg/kg over 5 weeks) and subcutaneous methotrexate (12 mg/m²/wk). Surprisingly, his PLCG2 phosphorylation at all 3 time points was not substantially different from the 16 treatment-naive patients (see e.g., FIG. 23A). Indeed, the PLCG2 phosphorylation of patient 7 (who was MDA5+ ) looked quite similar to the 9 p155/140 antibody-positive JDM patients (see e.g., FIG. 20B). In contrast, for reasons that are not yet clear, his p-MAPKAPK2 had normalized. This finding will be investigated in future studies and may provide insight into JDM response to therapy. To assess whether the inclusion of patient 7 in the treatment-naive cohort had skewed the results, individual 2-tailed Welch's t tests with Bonferroni correction (accounting for 897 comparisons between the 2 groups) were repeated without patient 7. This stringent statistical test identified that 3 of the top 4 most significant features (phosphoprotein in a specific immune cell sub-set) between the JDM patient and control groups were NK cell p-PLCG2 at 0, 3, and 15 minutes and that 7 of the 8 features that were significantly different between the groups involved p-PLCG2 (including all 3 time points in nonclassical monocytes), with the other significant feature involving p-MAPKAPK2 in nonclassical monocytes (data not shown)—nearly identical to the findings in the initial cohort (which included patient 7; see e.g., TABLE 7). Therefore, the inclusion of this new-onset patient (who had started therapy) did not substantially alter the results, and his results suggest that early therapy with corticosteroids and methotrexate is insufficient to attenuate the dysregulated NK cell p-PLCG2 seen in treatment-naive JDM.

Interestingly, several of the immune cell percentages or signaling differences in JDM patients (e.g., NK cell percentages in JDM patients) were partially attenuated with cessation of active disease. Indeed, PLCG2 and downstream MAPKAPK2 phosphorylation were substantially increased in NK cells of patients with clinically inactive disease in comparison with those from treatment-naive patients. For 7 of 11 paired treatment-naive patient and clinically inactive disease patient samples, CD16 receptor expression levels also increased with the cessation of active disease (see e.g., FIG. 16E). The attenuation of NK cell defects in patients with clinically inactive disease strongly suggests that the PLCG2 hypophosphorylation is not due to mutations in PLCG2.

The mechanism(s) underlying the NK cell PLCG2 hypophosphorylation in treatment-naive JDM patients is not yet clear. No differences were detected in total PLCG2 protein level (in a small subset of available patient samples). SHIP1, a negative regulator of p-PLCG2, was also assessed, and SHIP1 levels were actually lower in NK cells from treatment-naive JDM patients compared with controls, which would not explain the hypophosphorylation of PLCG2 in NK cells observed in these patients. CD16 expression was lower in JDM patients, but this appears to be correlative rather than causative because signaling through other NK cell receptors (2B4 and NKG2D with normal expression levels) manifested with decreased calcium flux. Future work will leverage RNA-Seq on sorted NK cells (from treatment-naive JDM patients and controls) to further delineate potential inhibitors and other PLCG2 signaling cascade components that contribute to differences in NK cell PLCG2 phosphorylation in the early, active JDM environment as well as the impact of the inflammatory environment in JDM on longitudinal changes in PLCG2 phosphorylation over the course of the disease. PLCG2 phosphorylation was also lower in treatment-naive JDM unstimulated classical monocytes.

Macrophage CSF-induced monocyte differentiation is mediated through PLCG2 phosphorylation. However, perturbations were not observed in the percentage of circulating monocytes in treatment-naive JDM patients compared to controls. Only a limited number of prior studies have examined classical monocytes in JDM. Future work will evaluate monocyte function to determine if hypophosphorylation of PLCG2 in classical monocytes has functional significance in these patients. 2 signaling defects in JDM were gained in this study, there were several limitations in this work, including the size of the treatment-naive patient cohort and the number of available PBMCs. The study included 17 treatment-naive patient samples from 2 medical centers and highlights the need for increased collaboration among pediatric centers to obtain enough patients to study new-onset, treatment-naive JDM patients in a statistically meaningful way. To maximize insights from small-volume patient samples, the mass cytometry samples were stimulated with a combination of different stimuli at 3 time points. However, the limited patient samples coupled with a paucity of NK cells in many of the JDM samples restricted the potential of follow-up experiments to assess the functional impact of hypophosphorylation of NK cells' PLCG2.

A better understanding of the etiology of JDM may inform new targeted therapeutic interventions (e.g., small molecules or biologics that target specific signaling pathways). This study highlighted the utility of mass cytometry coupled with multiparameter phospho-specific antibodies in identifying differences in signaling phenotype in small biological samples from treatment-naive JDM patients and controls. Treatment-naive JDM patient NK cells hypophosphorylated PLCG2, which resulted in decreased calcium flux, providing a mechanistic explanation for previous reports of poor NK cell killing in JDM patients. Future studies will focus on mechanisms underlying the NK cell PLCG2 signaling defects in new-onset, treatment-naive JDM patients and on potential strategies to mitigate this signaling defect.

### Example 3: Role of PLCG2 Hypophosphorylation in Juvenile Dermatomyositis (JDM)

This example describes studies designed to characterize the etiology of juvenile dermatomyositis (JDM) and the role of PLCG2 hypophosphorylation in mechanisms of the disease. Juvenile dermatomyositis (JDM) is the most common inflammatory myopathy of childhood. It manifests with a strong type I interferon signature in the peripheral blood and presents with characteristic skin rash and significant proximal muscle weakness. Despite the use of steroids and immunosuppressive therapies, JDM continues to inflict significant morbidity on children. Both adaptive and innate immune responses have been implicated in JDM pathogenesis, but the etiology of JDM was not well characterized. As described herein are experiments to understand how dysregulated innate immunity contributes to the etiology of JDM.

As described in Example 1, mass cytometry (CyTOF) was employed to investigate immune cell signaling in PBMCs from treatment-naïve JDM patients and healthy pediatric controls following in vitro stimulation (with a cocktail of cytokines, LPS, and crosslinking antibodies). Strikingly, 10 of the 12 signaling differences identified as stratifying between JDM patients and controls involved phospholipase C gamma-2 (PLCG2), a critical enzyme for NK cell and B cell signaling. PLCG2 hyperphosphorylation was observed in several clusters of CD4 and CD8 T cells. However, hypophosphorylation of NK cell PLCG2 was the primary signaling abnormality distinguishing JDM patients from controls. No differences were detected in upstream phosphorylation of Syk and ITK or in total PLCG2 protein levels in NK cells. The hypophosphorylation of NK cell PLCG2 was substantially normalized in patients in clinical remission, supporting a potential role for dysregulated NK cell PLCG2 signaling in JDM. Furthermore, studies demonstrated that suppressed PLCG2 phosphorylation in treatment-naïve JDM patient NK cells resulted in decreased calcium flux, suggesting that this signaling defect has functional consequences.

NK cells are innate immune cells that rapidly respond to viral infections as well as contribute to the suppression of inappropriate adaptive immune responses. They do this by making immunomodulatory cytokines (e.g., IFNγ or lL10) and by killing infected, transformed, or inappropriately activated cells. PLCG2 phosphorylation results in increased NK cell calcium flux with subsequent cytolytic granule movement and localization to the immune synapse, facilitating targeted NK cell-mediated cytotoxicity. Based on the data, it is presently thought that dysregulated PLCG2 signaling leads to suppressed NK cell functional responses in treatment-naïve JDM patients resulting in the loss of a potential brake on autoimmune T cells. As described herein, the following studies are disclosed to address these outstanding questions.

(1) Define the impact of PLCG2 hypophosphorylation on NK cell functional responses in treatment-naïve and remission JDM patients as well as adult DM patients in comparison to healthy controls.

(a) Perform mass cytometry profiling of NK cells (using a dedicated NK cell panel) from JDM and DM patients and controls to phenotypically characterize NK cell specific differences between JDM, DM, and controls.

(b) Characterize NK cell functional responses (e.g., degranulation, cytotoxicity, and cytokine production) from JDM patients prior to treatment and while in clinical remission in comparison to healthy pediatric controls.

(c) Quantify PLCG2 expression levels and phosphorylation, calcium flux, and NK cell functional responses in treatment-naïve adult-onset dermatomyositis (DM) patients at diagnosis and healthy adult controls to determine if the findings observed in JDM are generalizable to adult DM.

(2) Evaluate the influence of dysregulated PLCG2 signaling on T cells in treatment-naïve JDM patients

(a) Quantify PLCG2 phosphorylation, calcium flux, and functional responses in T cells from JDM patients as well as controls to discern if PLCG2 hyperphosphorylation increases the activation of T cells in JDM.

(b) Evaluate the ability of NK cells from JDM patients and healthy pediatric controls to kill autologous T cells to delineate the potential impact of dysregulated NK cell PLCG2 signaling in modulating NK cell targeted cytotoxicity in the suppression of adaptive immune responses in JDM.

(3) Investigate the mechanism(s) resulting in PLCG2 hypophosphorylation in JDM patients using RNA-seg as well as the evaluation of the impact of cytokine exposure on NK cell PLCG2 phosphorylation

(a) Interrogate transcriptional changes in the regulatory components of PLCG2 using RNA-seq on sorted NK cells, B cells, and CD8 and CD4 T cells from treatment-naïve and remission JDM patients as well as controls and validate the findings at the transcript and protein level.

(b) Investigate if exposure to IFNγ (and/or other cytokines elevated in the peripheral blood of treatment-naïve JDM patients) suppresses PLCG2 phosphorylation in NK cells from healthy pediatric controls.

(c) Determine if cytokines such as IL15 or IL2 can attenuate the hypophosphorylation of PLCG2 in treatment-naïve JDM patients (as a proof of principal regarding cytokine modulation of PLCG2 signaling defects in JDM).

In summary, it is presently thought that dysregulated PLCG2 signaling contributes to the development of JDM and studies are described herein to better define these signaling abnormalities. The disclosed studies have the potential to provide novel insight into the etiology of JDM and may facilitate new therapeutic interventions (e.g., IL-15 or blocking IFNα with a JAK inhibitor) to mitigate the impact of this autoimmune disease on children and potentially in adults with DM.

What is claimed is:

1. A method of restoring normal function and normal calcium flux in a dysfunctional natural killer (NK) cell comprising administering a therapeutically effective amount of a PLCG2 phosphorylation modulating agent comprising a cytokine to the dysfunctional NK cell, wherein the dysfunctional NK cell is from a subject having, suspected of having, or at risk of having a PLCG2 hypophosphorylation-associated disease, disorder, or condition.

2. The method of claim 1, wherein the dysfunctional NK cell has dysregulated PLCG2 signaling, PLCG2 haploinsufficiency, or PLCG2 hypophospohorylation.

3. The method of claim 1, wherein the PLCG2 hypophosphorylation-associated disease, disorder, or condition is PLCG2 haploinsufficiency (e.g., heterozygous loss-of-function mutation in PLCG2).

4. The method of claim 1, wherein the PLCG2 hypophosphorylation-associated disease, disorder, or condition is an autoimmune disease, an infectious disease, or an inflammatory disease, disorder, or condition (e.g., juvenile dermatomyositis (JDM), dermatomyositis (DM)).

5. The method of claim 1, wherein the PLCG2 hypophosphorylation-associated disease, disorder, or condition is a viral infection (e.g., herpesvirus, adenovirus, herpes simplex virus 1 (HSV1), cytomegalovirus (CMV)).

6. The method of claim 5, wherein the viral infection is a herpesvirus infection and the herpesvirus infection is an unusually severe or recurrent herpesvirus infection.

7. The method of claim 1, wherein the subject has herpesvirus infection susceptibility or bacterial infection susceptibility.

8. The method of claim 1, the dysfunctional NK cell is PLCG2 haploinsufficient and has a herpesvirus infection.

9. The method of claim 1, wherein the PLCG2 hypophosphorylation-associated disease, disorder, or condition is an inflammatory condition (e.g., multiple sclerosis (MS), systemic lupus erythematosus (SLE), rheumatoid arthritis (RA)).

10. The method of claim 1, wherein the cytokine is IL-2 or IL-15.

11. The method of claim 1, wherein the PLCG2 phosphorylation modulating agent restores normal NK cell function and normal NK cell calcium flux in the dysfunctional NK cell.

12. The method of claim 11, wherein restoring normal function and normal calcium flux, in the dysfunctional NK cell, results in improved cytotoxicity of the dysfunctional NK cell or improved ability of the dysfunctional NK cell to suppress inappropriate adaptive immune responses compared to an untreated dysfunctional NK cell.

13. A method of treating a subject in need thereof, comprising administering a PLCG2 phosphorylation modulating agent comprising a cytokine, wherein the subject has dysfunctional NK cells, wherein the subject has, is suspected of having, or is at risk of having a PLCG2 hypophosphorylation-associated disease, disorder, or condition.

14. The method of claim 13, wherein the dysfunctional NK cells have dysregulated PLCG2 signaling, PLCG2 haploinsufficiency, or PLCG2 hypophospohorylation.

15. The method of claim 13, wherein the PLCG2 hypophosphorylation-associated disease, disorder, or condition is PLCG2 haploinsufficiency (e.g., heterozygous loss-of-function mutation in PLCG2).

16. The method of claim 13, wherein the PLCG2 hypophosphorylation-associated disease, disorder, or condition is an autoimmune disease, an infectious disease, or an inflammatory disease, disorder, or condition (e.g., juvenile dermatomyositis (JDM), dermatomyositis (DM)).

17. The method of claim 13, wherein the PLCG2 hypophosphorylation-associated disease, disorder, or condition is a viral infection (e.g., herpesvirus, adenovirus, herpes simplex virus 1 (HSV1), cytomegalovirus (CMV)).

18. The method of claim 17, wherein the viral infection is a herpesvirus infection and the herpesvirus infection is an unusually severe or recurrent herpesvirus infection.

19. The method of claim 13, wherein the subject has herpesvirus infection susceptibility or bacterial infection susceptibility.

20. The method of claim 13, the dysfunctional NK cells are PLCG2 haploinsufficient and have a herpesvirus infection.

21. The method of claim 13, wherein the PLCG2 hypophosphorylation-associated disease, disorder, or condition is an inflammatory condition (e.g., multiple sclerosis (MS), systemic lupus erythematosus (SLE), rheumatoid arthritis (RA)).

22. The method of claim 13, wherein the cytokine is IL-2 or IL-15.

23. The method of claim 13, wherein the PLCG2 phosphorylation modulating agent restores normal NK cell function and normal NK cell calcium flux in the dysfunctional NK cells.

24. The method of claim 13, wherein restoring normal NK cell function and normal NK cell calcium flux results in improved NK cell-mediated cytotoxicity, suppression of inappropriate adaptive immune responses, or reduced autoimmunity in the subject when compared to an untreated subject.
